(19) United States
(12) Patent Application Publication
(10) Pub. No.: US 2023/0406864 A1
(43) Pub. Date: Dec. 21, 2023

(54) 7-AZOLE SUBSTITUTED 2-AMINOQUINAZOLINE INHIBITORS OF HPK1

(71) Applicant: MERCK SHARP & DOHME LLC, Rahway, NJ (US)

(72) Inventors: Joanna L. Chen, Braintree, MA (US); Peter H. Fuller, Ashland, MA (US); Joey L. Methot, Westwood, MA (US); Elsie C. Yu, Brighton, MA (US)

(73) Assignee: MERCK SHARP & DOHME LLC, Rahway, NJ (US)

(21) Appl. No.: 18/250,429

(22) PCT Filed: Nov. 4, 2021
(86) PCT No.: PCT/US21/57969

§ 371 Date: Apr. 25, 2023

Related U.S. Application Data
(60) Provisional application No. 63/111,385, filed on Nov. 9, 2020.

(51) Int. Cl. C07D 498/04; C07D 519/00

(52) U.S. Cl. CPC C07D 498/04; C07D 519/00

(57) ABSTRACT

Compounds of the following formula (I); or the pharmaceutically acceptable salts thereof, are inhibitors of haematopoietic progenitor kinase 1 (HPK1) useful in the treatment of diseases or disorders associated with HPK1. Also disclosed herein are uses of these compounds in the potential treatment or prevention of an HPK1-associated disease or disorder. Also disclosed herein are compositions comprising one or more of the compounds. Further disclosed herein are uses of these compositions in the potential prevention or treatment of an HPK1-associated disease or disorder.

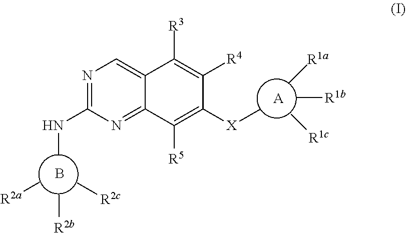

### BACKGROUND OF THE INVENTION

Cancer immunotherapy is treatment that uses the human body's own immune system to help fight cancer. This unique approach has witnessed significant clinical successes in the treatment of a variety of tumor types in recent years, particularly with the application of immune checkpoint inhibitors and chimeric antigen T cell therapy. Two of the most investigated checkpoint blockades (i.e., CTLA4 and PD-1 inhibitors) have demonstrated remarkable antitumor activity by overcoming immunosuppressive mechanisms at the tumor site. CTLA4 blockade predominantly enhances T cell activation during the priming phase of the immune response, whereas PD-1 inhibitors appear to release exhausted but otherwise activated effector T cell populations and reduce regulatory T cell function. While these monoclonal antibody-based T cell interventions have proven to be effective, utility of this approach is limited as they can only target receptors on the cell surface. To the contrary, small-molecule T cell activators offer the opportunity to target both extracellular and intracellular immune targets including kinases. Furthermore, inhibiting immune suppressive kinases has the potential to directly activate T cells, thus bypassing checkpoint inhibitory pathways and overcoming intrinsic and acquired resistance to checkpoint receptor blockade.

Haematopoietic progenitor kinase 1 (HPK1; also known as MAP4K1) is a member of the germinal center kinase family of serine/threonine kinases and is mainly expressed by haematopoietic cells. In T cells, it is believed that HPK1 phosphorylates serine 376 of SLP76 after T cell receptor (TCR) triggers and induces the association of SLP76 with 14-3-3 proteins. Knockdown of HPK1 expression in Jurkat T cells has been shown to increase TCR-induced activation of the IL2 gene. Further, antigen-stimulated T cells from HPK1-deficient mice proliferated more vigorously and produced higher amounts of cytokines as compared to antigen-stimulated T-cells from wild-type mice. Importantly, HPK1-deficient mice developed a more severe form of experimental autoimmune encephalomyelitis (EAE), a model of multiple sclerosis. Both in vitro and in vivo, HPK1 knockout dendritic cells (DC) have demonstrated enhanced antigen presentation function. Particularly, both HPK1 knockout T cells and HPK1 knockout DCs have been implicated in tumor rejection in a murine model of lung cancer. These findings have validated HPK1 as a novel target for anti-cancer immunotherapy. Inhibition of HPK1 with small molecule inhibitors therefore has the potential to be a treatment for cancers and other disorders. Compounds disclosed herein are useful in the potential treatment or prevention of HPK1-related diseases.

### SUMMARY OF THE INVENTION

Compounds of the formula I:

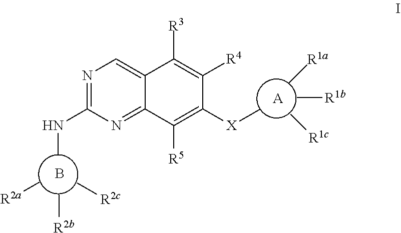

or pharmaceutically acceptable salts thereof, are inhibitors of haematopoietic progenitor kinase 1 (HPK1) useful in the treatment of diseases or disorders associated with HPK1. Also disclosed herein are uses of these compounds in the potential treatment or prevention of an HPK1-associated disease or disorder. Also disclosed herein are compositions comprising one or more of the compounds. Further disclosed herein are uses of these compositions in the potential prevention or treatment of an HPK1-associated disease or disorder.

### DETAILED DESCRIPTION OF THE INVENTION

The present invention is directed to compounds of the formula I:

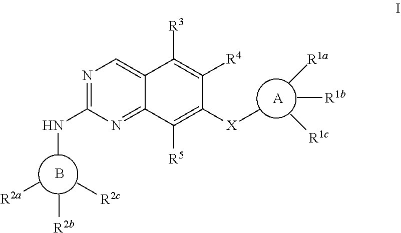

wherein:
A is a phenyl, cycloalkyl, or heterocyclyl ring;
B is a pyrazolyl or thiazolyl ring;
X is a bond, —O—, —O(C₁₋₃alkyl)-, —NH—, —NH(C₁₋₃alkyl)- or —N(CH₃)(C₁₋₃alkyl)-;
R^{1a}, R^{1b} and R^{1c} as are present are independently selected from:
   (1) hydrogen,
   (2) halogen.
   (3) hydroxyl,
   (4) C₁₋₆alkyl, which is unsubstituted or substituted with substituents selected from: hydroxy, —NH₂, —(CO)NH(C₁₋₆alkyl), —CN, and fluoro,
   (5) —O—C₁₋₆alkyl, which is unsubstituted or substituted with fluoro,
   (6) —C₃₋₆cyclolkyl, which is unsubstituted or substituted with substituents selected from: hydroxy, methoxy, —(CO)NH(C₁₋₆alkyl), —(CO)O(C₁₋₆alkyl), —CN, and fluoro,
   (7) C₂₋₆alkynyl,
   (8) —NH₂,
   (9) —NH(C₁₋₆alkyl),
   (10) —N(C₁₋₆alkyl)₂,
   (11) —(CO)(C₁₋₆alkyl),
   (12) —(CO)NH₂,
   (13) —(CO)NH(C₁₋₆alkyl),
   (14) —(CO)NH(C₃₋₆cycloalkyl),
   (15) —NH(CO)(C₁₋₆alkyl),
   (16) —SO₂—C₁₋₆alkyl,
   (17) —NH—SO₂—C₁₋₆alkyl,
   (18) —CN,
   (19) keto,
   (20) -phenyl,
   (21) -pyridyl,
   (22) -diazolyl,
   (23) -morpholinyl,
   (24) -oxazolyl,
   (25) -oxadiazolyl,
   (26) -piperazinyl,
   (27) -piperidinyl, and
   (28) -thiazolyl.
   or R^{1a} and R^{1b} may be joined to form a 1,4-dioxanyl, 1,3-dioxolanyl, morpholinyl, oxazolyl, piperidinyl, pyrazolyl, pyrrolidinyl, tetrahydropyranyl, tetrahydroquinolinyl, or thiazolyl ring;
R^{2a}, R^{2b} and R^{2c} as are present are independently selected from:
   (1) hydrogen,
   (2) halogen,
   (3) hydroxyl,
   (4) C₁₋₆alkyl, which is unsubstituted or substituted with substituents selected from: hydroxy, —(CO)NH(C₁₋₆alkyl), —(CO)O(C₁₋₆alkyl), —SO₂—C₁₋₆alkyl, C₃₋₆cycloalkyl, —CN, phenyl and fluoro,
   (5) —O—C₁₋₆alkyl, which is unsubstituted or substituted with fluoro,
   (6) —C₃₋₆cycloalkyl, which is unsubstituted or substituted with substituents selected from: hydroxy, methoxy, —C₁₋₆alkyl, —(CO)NH(C₁₋₆alkyl), —(CO)O(C₁₋₆alkyl), —CN, and fluoro,
   (7) —NH₂,
   (8) —NH(C₁₋₆alkyl),
   (9) —N(C₁₋₆alkyl)₂,
   (10) —(CO)(C₁₋₆alkyl),
   (11) —(CO)NH₂,
   (12) —(CO)NH(C₁₋₆alkyl),
   (13) —NH(CO)(C₁₋₆alkyl),
   (14) —SO₂—C₁₋₆alkyl,
   (15) —SO₂—NH(C₁₋₆alkyl),
   (16) —SO₂—N(C₁₋₆alkyl)₂,
   (17) azaspiro[3.3]heptanyl, which is unsubstituted or substituted with —C₁₋₆alkyl or oxetanyl, which is unsubstituted or substituted with —C₁₋₆alkyl,
   (18) azetidinyl, which is unsubstituted or substituted with —C₁₋₆alkyl or —(CO)O(C₁₋₆alkyl),
   (19) bicyclopentyl, which is unsubstituted or substituted with —C₁₋₆alkyl or —(CO)O(C₁₋₆alkyl),
   (20) oxazolyl,
   (21) oxadiazolyl,
   (22) oxetanyl, which is unsubstituted or substituted with —C₁₋₆alkyl,
   (23) piperidinyl, which is unsubstituted or substituted with —C₁₋₆alkyl or oxetanyl, which is unsubstituted or substituted with —C₁₋₆alkyl,
   (24) pyrrolidinyl, which is unsubstituted or substituted with oxo or —C₁₋₆alkyl, and
   (25) thiazolyl,
   or R^{2b} and R^{2c} may be joined to form a pyrrolyl, dihydrospiro[1,4′-pyrazolo[1,5-d][1,4]diazepin]-7′(8′H)-one, or dihydro-4H-pyrazolo[1,5-d][1,4]diazepin-one ring, which is unsubstituted or substituted with —C₁₋₆alkyl, —C₁₋₆alkyl-OH or —C₃₋₆cycloalkyl;
R³ is selected from:
   (1) hydrogen,
   (2) —NH₂,
   (3) chloro, and
   (4) fluoro;
R⁴ is selected from:
   (1) hydrogen,
   (2) cyano,
   (3) chloro, and
   (4) fluoro;
R⁵ is selected from:
   (1) hydrogen,
   (2) methyl,
   (3) cyano,
   (4) chloro,
   (5) fluoro, and
   (6) bromo;
or a pharmaceutically acceptable salt thereof.

An embodiment of the present invention includes compounds of the formula Ia:

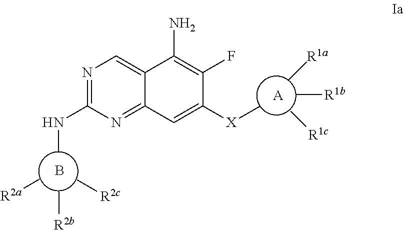

wherein A, B, X, R^{1a}, R^{1b}, R^{1c}, R^{2a}, R^{2b} and R^{2c} are defined herein; or a pharmaceutically acceptable salt thereof.

An embodiment of the present invention includes compounds of the formula Ib:

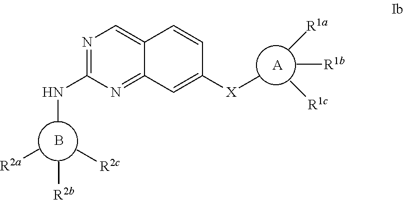

wherein A, B, X, R^{1a}, R^{1b}, R^{1c}, R^{2a}, R^{2b} and R^{2c} are defined herein; or a pharmaceutically acceptable salt thereof.

An embodiment of the present invention includes compounds of the formula II:

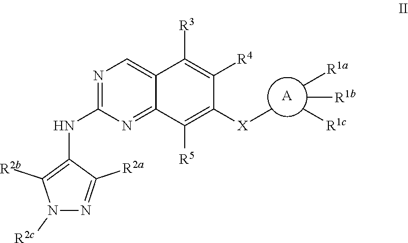

wherein A, X, R^{1a}, R^{1b}, R^{1c}, R^{2a}, R^{2b}, R^{2c}, R³, R⁴ and R⁵ are defined herein; or a pharmaceutically acceptable salt thereof.

An embodiment of the present invention includes compounds of the formula IIa:

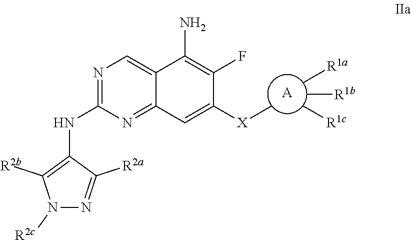

wherein A, X, R^{1a}, R^{1b}, R^{1c}, R^{2a}, R^{2b} and R^{2c} are defined herein; or a pharmaceutically acceptable salt thereof.

An embodiment of the present invention includes compounds of the formula IIb:

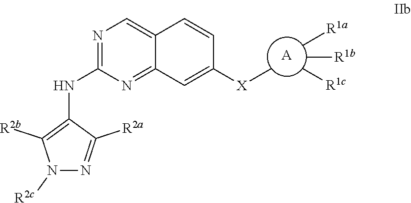

wherein A, X, R^{1a}, R^{1b}, R^{1c}, R^{2a}, R^{2b} and R^{2c} are defined herein; or a pharmaceutically acceptable salt thereof.

An embodiment of the present invention includes compounds of the formula III:

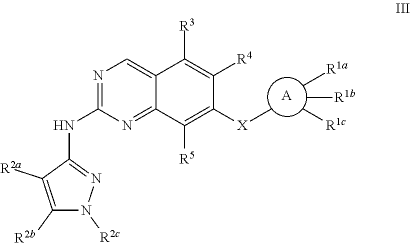

wherein A, X, R^{1a}, R^{1b}, R^{1c}, R^{2a}, R^{2b}, R^{2c}, R³, R⁴ and R⁵ are defined herein; or a pharmaceutically acceptable salt thereof.

An embodiment of the present invention includes compounds of the formula IIIa:

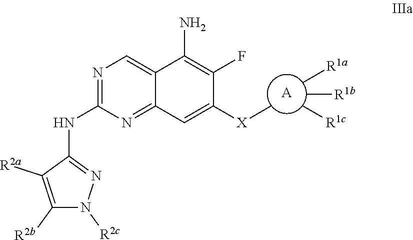

wherein A, X, R^{1a}, R^{1b}, R^{1c}, R^{2a}, R^{2b} and R^{2c} are defined herein; or a pharmaceutically acceptable salt thereof.

An embodiment of the present invention includes compounds of the formula IIIb:

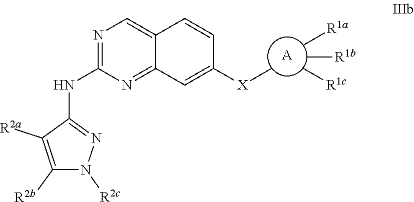

wherein A, X, R^{1a}, R^{1b}, R^{1c}, R^{2a}, R^{2b} and R^{2c} are defined herein; or a pharmaceutically acceptable salt thereof.

An embodiment of the present invention includes compounds of the formula IV:

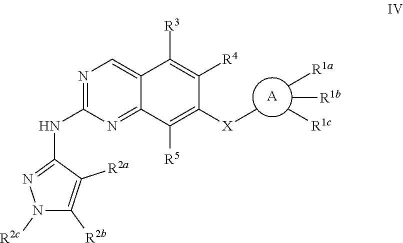

wherein A, X, R^{1a}, R^{1b}, R^{1c}, R^{2a}, R^{2b}, R^{2c}, R³, R⁴ and R⁵ are defined herein; or a pharmaceutically acceptable salt thereof.

An embodiment of the present invention includes compounds of the formula IVa:

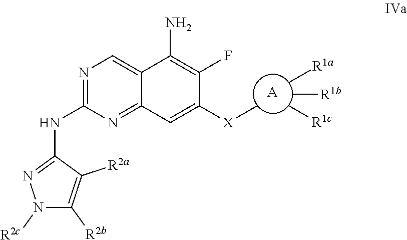

wherein A, X, R^{1a}, R^{1b}, R^{1c}, R^{2a}, R^{2b} and R^{2c} are defined herein; or a pharmaceutically acceptable salt thereof.

An embodiment of the present invention includes compounds of the formula IVb:

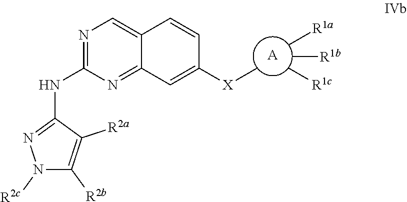

wherein A, X, R^{1a}, R^{1b}, R^{1c}, R^{2a}, R^{2b} and R^{2c} are defined herein; or a pharmaceutically acceptable salt thereof.

An embodiment of the present invention includes compounds of the formula V:

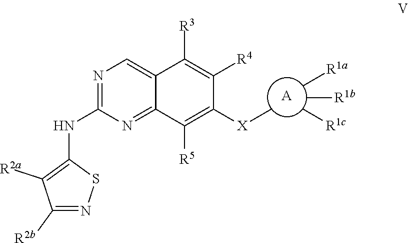

wherein A, X, R^{1a}, R^{1b}, R^{1c}, R^{2a}, R^{2b}, R³, R⁴ and R⁵ are defined herein; or a pharmaceutically acceptable salt thereof.

An embodiment of the present invention includes compounds of the formula Va:

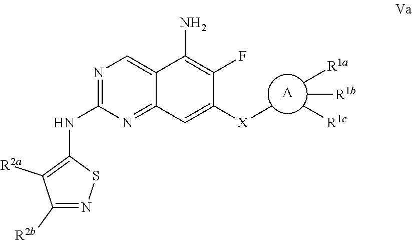

wherein A, X, R^{1a}, R^{1b}, R^{1c}, R^{2a} and R^{2b} are defined herein; or a pharmaceutically acceptable salt thereof.

An embodiment of the present invention includes compounds of the formula Vb.

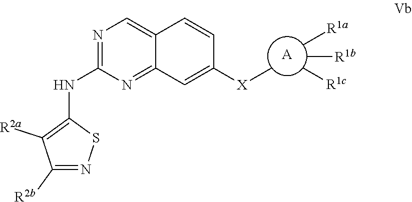

wherein A, X, R^{1a}, R^{1b}, R^{1c}, R^{2a} and R^{2b} are defined herein; or a pharmaceutically acceptable salt thereof.

An embodiment of the present invention includes compounds wherein A is a phenyl, pyridyl, or 2,3-dihydro-1H-pyrido[2,3-b][1,4]oxazin-7-yl ring. An embodiment of the present invention includes compounds wherein A is a phenyl ring. An embodiment of the present invention includes compounds wherein A is a pyridyl ring. An embodiment of the present invention includes compounds wherein A is a 2,3-dihydro-1H-pyrido[2,3-b][1,4]oxazin-7-yl ring.

An embodiment of the present invention includes compounds wherein B is a pyrazolyl ring. An embodiment of the present invention includes compounds wherein B is a pyazol-4-yl ring. An embodiment of the present invention includes compounds wherein B is a 1H-pyazol-4-yl ring. An embodiment of the present invention includes compounds wherein B is a thiazolyl ring. An embodiment of the present invention includes compounds wherein B is a 1,2-thiazol-5-yl ring.

An embodiment of the present invention includes compounds wherein X is a bond, —O—, or —O(CH₂)—. An embodiment of the present invention includes compounds wherein X is a bond. An embodiment of the present invention includes compounds wherein X is —O—. An embodiment of the present invention includes compounds wherein X is —O(CH₂)—.

An embodiment of the present invention includes compounds wherein R^{1a}, R^{1b} and R^{1c} as are present are independently selected from:
(1) hydrogen,
(2) fluoro,
(3) chloro,
(4) hydroxyl,
(5) C₁₋₃alkyl, which is unsubstituted or substituted with hydroxy or one or more fluoro,
(6) —O—C₁₋₃alkyl, which is unsubstituted or substituted with one or more fluoro,
(7) C₃₋₆cycloalkyl,
(8) —NH₂,
(9) —NH(C₁₋₃alkyl),
(10) —N(C₁₋₃alkyl)₂,
(11) keto, and
(12) -phenyl.

An embodiment of the present invention includes compounds wherein R^{1a}, R^{1b} and R^{1c} as are present are independently selected from:
(1) hydrogen,
(2) fluoro,
(3) hydroxyl,
(4) —CH₃,
(5) —CHF₂,
(6) —CF₃,
(7) —CH₂OH,
(8) —CH₂CH₃,
(9) —C(CH₃)OH,
(10) —OCH₃,
(11) —OCHF₂,
(12) —OCH₂CH₂F,
(13) —N(CH₃)₂,
(14) cyclopropyl, and
(15) phenyl.

An embodiment of the present invention includes compounds wherein R^{1c} is hydrogen and R^{1a} and R^{1b}, as are present, are independently selected from:
(1) hydrogen,
(2) fluoro,
(3) hydroxyl,
(4) —CH₃,
(5) —CHF₂,
(6) —CF₃,
(7) —CH₂OH,
(8) —CH₂CH₃,
(9) —C(CH₃)OH,
(10) —OCH₃,
(11) —OCHF₂,
(12) —OCH₂CH₂F,
(13) —N(CH₃)₂,
(14) cyclopropyl, and
(15) phenyl.

An embodiment of the present invention includes compounds wherein R^{1c} is hydrogen, and R^{1a} and R^{1b} may be joined to form a morpholinyl ring.

An embodiment of the present invention includes compounds wherein R^{1c} is methyl, and R^{1a} and R^{1b} may be joined to form a morpholinyl ring.

An embodiment of the present invention includes compounds wherein R^{2a}, R^{2b} and R^{2c} as are present are independently selected from:
(1) hydrogen,
(2) halogen,
(3) hydroxyl,
(4) C₁₋₆alkyl, and
(5) —O—C₁₋₆alkyl.

An embodiment of the present invention includes compounds wherein R^{2a} is hydrogen, R^{2b} is hydrogen or —C₁₋₆alkyl, and R^{2c} is independently selected from:
(1) C₁₋₆alkyl, which is unsubstituted or substituted with substituents selected from: hydroxy, —(CO)NH(C₁₋₆alkyl), —(CO)O(C₁₋₆alkyl), —C₃₋₆cycloalkyl, phenyl and fluoro,
(2) —C₃₋₆cycloalkyl, which is unsubstituted or substituted with substituents selected from: hydroxy, methoxy, —C₁₋₆alkyl, —(CO)NH(C₁₋₆alkyl), —(CO)O(C₁₋₆alkyl), and fluoro,
(3) azaspiro[3.3]heptanyl, which is unsubstituted or substituted with —C₁₋₆alkyl or oxetanyl, which is unsubstituted or substituted with —C₁₋₆alkyl,
(4) azetidinyl, which is unsubstituted or substituted with —C₁₋₆alkyl or —(CO)O(C₁₋₆alkyl),
(5) bicyclopentyl, which is unsubstituted or substituted with —C₁₋₆alkyl or —(CO)O(C₁₋₆alkyl),
(6) oxazolyl,
(7) oxadiazolyl,
(8) oxetanyl, which is unsubstituted or substituted with —C₁₋₆alkyl,
(9) piperidinyl, which is unsubstituted or substituted with —C₁₋₆alkyl or oxetanyl, which is unsubstituted or substituted with —C₁₋₆alkyl,
(10) pyrrolidinyl, which is unsubstituted or substituted with oxo or —C₁₋₆alkyl, and
(11) thiazolyl.

An embodiment of the present invention includes compounds wherein R^{2a} is hydrogen, and R^{2b} and R^{2c} are joined to form a pyrrolyl, dihydrospiro[1,4′-pyrazolo[1,5-d][1,4]diazepin]-7′(8′H)-one, or dihydro-4H-pyrazolo[1,5-d][1,4]diazepin-one ring, which is unsubstituted or substituted with —C₁₋₆alkyl, —C₁₋₆alkyl-OH or —C₃₋₆cycloalkyl.

An embodiment of the present invention includes compounds wherein R^{2a} is hydrogen. An embodiment of the present invention includes compounds wherein R^{2b} is hydrogen.

An embodiment of the present invention includes compounds wherein R³ is hydrogen. An embodiment of the present invention includes compounds wherein R³ is —NH₂.

An embodiment of the present invention includes compounds wherein R⁴ is hydrogen or fluoro. An embodiment of the present invention includes compounds wherein R⁴ is hydrogen. An embodiment of the present invention includes compounds wherein R⁴ is chloro. An embodiment of the present invention includes compounds wherein R⁴ is fluoro. An embodiment of the present invention includes compounds wherein R⁴ is cyano.

An embodiment of the present invention includes compounds wherein R⁵ is hydrogen or chloro. An embodiment of the present invention includes compounds wherein R⁵ is hydrogen. An embodiment of the present invention includes compounds wherein R⁵ is chloro.

Certain embodiments of the present invention include a compound which is selected from the group consisting of the subject compounds of the Examples herein or a pharmaceutically acceptable salt thereof.

Certain embodiments of the present invention include a compound which is selected from the group consisting of;
7-(8-methyl-2,3-dihydro-1H-pyrido[2,3-b][1,4]oxazin-7-yl)-N-[1-(oxetan-3-yl)-1H-pyrazol-4-yl]quinazolin-2-amine;
7-(8-methyl-2,3-dihydro-1H-pyrido[2,3-b][1,4]oxazin-7-yl)-N-{1-[2-(3-methyloxetan-3-yl)-2-azaspiro[3.3]heptan-6-yl]-1H-pyrazol-4-yl}quinazolin-2-amine;
1-[3-(4-{[7-(8-methyl-2,3-dihydro-1H-pyrido[2,3-b][1,4]oxazin-7-yl)quinazolin-2-yl]amino}-1H-pyrazol-1-yl)azetidin-1-yl]propan-1-one;
6′-methyl-2′-{[7-(8-methyl-2,3-dihydro-1H-pyrido[2,3-b][1,4]oxazin-7-yl)quinazolin-2-yl]amino}-5′,6′-dihydrospiro[cyclopropane-1,4′-pyrazolo[1,5-d][1,4]diazepin]-7′(8′H)-one;
7-(8-methyl-2,3-dihydro-1H-pyrido[2,3-b][1,4]oxazin-7-yl)-N-(1-methyl-1H-pyrazol-4-yl)quinazolin-2-amine;
2-{[7-(8-methyl-2,3-dihydro-1H-pyrido[2,3-b][1,4]oxazin-7-yl)quinazolin-2-yl]amino}-6-(propan-2-yl)-5,6-dihydro-4H-pyrazolo[1,5-d][1,4]diazepin-7(8H)-one;
2-{[7-(8-methyl-2,3-dihydro-1H-pyrido[2,3-b][1,4]oxazin-7-yl)quinazolin-2-yl]amino}-6-(propan-2-yl)-7,8-dihydro-4H-pyrazolo[1,5-d][1,4]diazepin-5(6H)-one;
6-(2-hydroxy-2-methylpropyl)-2-((7-(8-methyl-2,3-dihydro-1H-pyrido[2,3-b][1,4]-oxazin-7-yl)quinazolin-2-yl)amino)-7,8-dihydro-4H-pyrazolo[1,5-d][1,4]diazepin-5(6H)-one;
1-[3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)azetidin-1-yl]propan-1-one;
6-fluoro-7-(8-methyl-2,3-dihydro-1H-pyrido[2,3-b][1,4]oxazin-7-yl)-N˜2˜-{1-[2-(3-methyloxetan-3-yl)-2-azaspiro[3.3]heptan-6-yl]-1H-pyrazol-4-yl}quinazoline-2,5-diamine;
6-fluoro-7-(8-methyl-2,3-dihydro-1H-pyrido[2,3-b][1,4]oxazin-7-yl)-N˜2˜-{1-[1-(3-methyloxetan-3-yl)piperidin-4-yl]-1H-pyrazol-4-yl}quinazoline-2,5-diamine;
N˜2˜-[1-(2-azaspiro[3.3]heptan-6-yl)-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-[1-(azetidin-3-yl)-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-[1-(cyclopropylmethyl)-1H-pyrazol-4-yl]-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[1-(2,2,2-trifluoroethyl)-1H-pyrazol-4-yl]quinazoline-2,5-diamine;
6-fluoro-N˜2˜-[1-(1-methylcyclopropyl)-1H-pyrazol-4-yl]-7-(8-methyl-2,3-dihydro-1H-pyrido[2,3-b][1,4]oxazin-7-yl)quinazoline-2,5-diamine;
N˜2˜-(1-cyclopropyl-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-(1-methyl-1H-pyrazol-4-yl)quinazoline-2,5-diamine;
6-fluoro-7-(8-methyl-2,3-dihydro-1H-pyrido[2,3-b][1,4]oxazin-7-yl)-N˜2˜-[1-(piperidin-4-yl)-1H-pyrazol-4-yl]quinazoline-2,5-diamine;
N˜2˜-(1-benzyl-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[1-(2-methylpropyl)-1H-pyrazol-4-yl]quinazoline-2,5-diamine;
6-fluoro-7-(8-methyl-2,3-dihydro-1H-pyrido[2,3-b][1,4]oxazin-7-yl)-N˜2˜-[1-(propan-2-yl)-1H-pyrazol-4-yl]quinazoline-2,5-diamine;
methyl 3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)bicyclo[1.1.1]pentane-1-carboxylate;
N˜2˜-(1-ethyl-5-methyl-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-(3-methyl-1,2-thiazol-5-yl)quinazoline-2,5-diamine;
1-(4-{[5-amino-6-fluoro-7-(8-methyl-2,3-dihydro-1H-pyrido[2,3-b][1,4]oxazin-7-yl)quinazolin-2-yl]amino}-5-chloro-1H-pyrazol-1-yl)-2-methylpropan-2-ol;
3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-1-methylpyrrolidin-2-one;
2-{[5-amino-6-fluoro-7-(8-methyl-2,3-dihydro-1H-pyrido[2,3-b][1,4]oxazin-7-yl)quin-azolin-2-yl]amino}-6-(propan-2-yl)-7,8-dihydro-4H-pyrazolo[1,5-d][1,4]diazepin-5(6H)-one;
3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-1-methylpyrrolidin-2-one;
N˜2˜-(1-ethyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-(1-cyclobutyl-1H-pyrazol-4-yl)-6-fluoro-7-(8-methyl-2,3-dihydro-1H-pyrido[2,3-b][1,4]oxazin-7-yl)quinazoline-2,5-diamine;
1-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-2-methylpropan-2-ol;
N˜2˜-(5-chloro-1-cyclopropyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-[5-chloro-1-(1-methylcyclopropyl)-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-(1-ethyl-3-methyl-1H-pyrazol-4-yl)-6-fluoro-7-(8-methyl-2,3-dihydro-1H-pyrido[2,3-b][1,4]oxazin-7-yl)quinazoline-2,5-diamine;
3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-N-methylbicyclo[1.1.1]pentane-1-carboxamide;
N˜2˜-[1-(difluoromethyl)-5-methyl-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-(1,5-dimethyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-[1-(2,2-difluoroethyl)-3-methyl-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-[1-(2,2-difluoroethyl)-1H-pyrazol-4-yl]-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[3-methyl-1-(propan-2-yl)-1H-pyrazol-4-yl]quinazoline-2,5-diamine;
N˜2˜-(1,3-dimethyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-(5,6-dihydro-4H-pyrrolo[1,2-b]pyrazol-3-yl)-6-fluoro-7-(8-methyl-2,3-dihydro-1H-pyrido[2,3-b][1,4]oxazin-7-yl)quinazoline-2,5-diamine;
N˜2˜-(4-chloro-3-methyl-1,2-thiazol-5-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-(4-methyl-1,2-thiazol-5-yl)quinazoline-2,5-diamine;
N˜2˜-(1-ethyl-5-fluoro-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methy-2,3-dihydro-1H-pyrido[2,3-b][1,4]oxazin-7-yl)-N˜2˜-[1-(oxetan-3-yl)-1H-pyrazol-4-yl]quinazoline-2,5-diamine;
2-(4-{[5-amino-6-fluoro-7-(8-methyl-2,3-dihydro-1H-pyrido[2,3-b][1,4]oxazin-7-yl)quinazolin-2-yl]amino}-3-methyl-1H-pyrazol-1-yl)-N-methylacetamide;
2-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-N,2-dimethylpropanamide;
6-fluoro-7-(8-methyl-2,3-dihydro-1H-pyrido[2,3-b][1,4]oxazin-7-yl)-N2-(1-((methylsulfonyl)methyl)-1H-pyrazol-4-yl)quinazoline-2,5-diamine;
8-chloro-6-fluoro-N˜2˜-[1-(1-methylcyclopropyl)-1H-pyrazol-4-yl]-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[1-(1-methylpiperidin-4-yl)-1H-pyrazol-4-yl]quinazoline-2,5-diamine;
N-˜2-(1-cyclopropyl-5-methyl-1H-pyrazol-4-yl)-6-fluoro-7-(8-methyl-2,3-dihydro-1H-pyrido[2,3-b][1,4]oxazin-7-yl)quinazoline-2,5-diamine;
2-[3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)bicyclo[1.1.1]pentan-1-yl]propan-2-ol;
(S)-6-fluoro-7-(8-methyl-2,3-dihydro-1H-pyrido[2,3-b][1,4]oxazin-7-yl)-N2-(1-(1-methylpyrrolidin-3-yl)-1H-pyrazol-4-yl)quinazoline-2,5-diamine; and
(R)-6-fluoro-7-(8-methyl-2,3-dihydro-1H-pyrido[2,3-b][1,4]oxazin-7-yl)-N2-(1-(1-methylpyrrolidin-3-yl)-1H-pyrazol-4-yl)quinazoline-2,5-diamine;
   or a pharmaceutically acceptable salt thereof.

In one embodiment, the present invention is a composition comprising a compound of formula I, II, IIa, IIb, IIc, IId, III, IIIa, IIIb, IIIc, IIId, IV, IVa, IVb, or a pharmaceutically acceptable salt thereof, and at least one pharmaceutically acceptable carrier.

In another embodiment, the present invention is a method of treating cancer, metastasis, inflammation and auto-immune pathogenesis comprising administering to a patient in need thereof a composition of formula I, IIa, IIb, IIc, IId, III, IIIa, IIIb, IIIc, IIId, IV, IVa, IVb, or a pharmaceutically acceptable salt thereof.

In another embodiment, the present invention is the use of a compound of formula I, II, IIa, IIb, IIc, IId, III, IIIa, IIIb, IIIc, IIId, IV, IVa, IVb, or a pharmaceutically acceptable salt thereof, in the manufacture of a medicament for treating cancer, metastasis, inflammation and auto-immune pathogenesis.

In another embodiment, the present invention includes compounds of formula I, II, IIa, IIb, IIc, IId, III, IIIa, IIIb, IIIc, IIId, IV, IVa, IVb, or a pharmaceutically acceptable salt thereof, for use in the treatment of cancer, metastasis, inflammation and auto-immune pathogenesis.

Also disclosed herein is a method of inhibiting activity of haematopoietic progenitor kinase 1 (HPK1) comprising contacting HPK1 with a compound disclosed herein, or a pharmaceutically acceptable salt thereof.

Also disclosed herein is a method of treating cancer, metastasis, inflammation and auto-immune pathogenesis, comprising administering to a patient suffering from at least one of said diseases or disorder an effective amount of a compound disclosed herein, or a pharmaceutically acceptable salt thereof.

Also disclosed herein is a method of treating melanoma in a patient comprising administering to said patient an effective amount of a compound disclosed herein, or a pharmaceutically acceptable salt thereof.

The present invention relates to compounds and compositions that are capable of inhibiting the activity of HPK1. The invention features methods of treating, preventing or ameliorating a disease or disorder in which HPK1 plays a role by administering to a patient in need thereof a therapeutically effective amount of a compound of Formula (I), or a pharmaceutically acceptable salt thereof. The invention also features methods of treating, preventing or ameliorating a disease or disorder in which HPK1 plays a role by administering to a patient in need thereof a therapeutically effective amount of a compound of Formula (II), or a pharmaceutically acceptable thereof. The methods of the present invention can be used in the treatment of a variety of HPK1 dependent diseases and disorders by inhibiting the activity of HPK1 enzymes. Inhibition of HPK1 provides a novel approach to the treatment, prevention, or amelioration of diseases including, but not limited to, cancer and metastasis

Further disclosed herein is a compound disclosed herein, or a pharmaceutically acceptable salt thereof, for use in therapy. In one embodiment, disclosed herein is the use of a compound disclosed herein, or a pharmaceutically acceptable salt thereof, for the preparation of a medicament for use in therapy.

“Alkyl” refers to both branched- and straight-chain saturated aliphatic hydrocarbon groups of 1 to 18 carbon atoms, or more specifically, 1 to 12 carbon atoms. Examples of such groups include, but are not limited to, methyl (Me), ethyl (Et), n-propyl (Pr), n-butyl (Bu), n-pentyl, n-hexyl, and the isomers thereof such as isopropyl (i-Pr), isobutyl (i-Bu), sec-butyl (s-Bu), tert-butyl (t-Bu), isopentyl, and isohexyl. Alkyl groups may be optionally substituted with one or more substituents as defined herein. “C₁₋₆alkyl” refers to an alkyl group as defined herein having 1 to 6 carbon atoms.

“Cycloalkyl” refers to a non-aromatic ring system comprising from 3 to 6 ring carbon atoms. Non-limiting examples of monocyclic cycloalkyl include cyclopropyl, cyclobutyl, cyclopentyl. Non-limiting examples of cycloalkyl additionally include bicyclic spiro-cycloalkyl including spirohexane

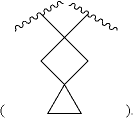 [SMILES: *C1(*)CC2(CC2)C1]

“Aryl” refers to an aromatic monocyclic or multicyclic ring moiety comprising 6 to 14 ring carbon atoms, or more specifically, 6 to 10 ring carbon atoms. Monocyclic aryl rings include, but are not limited to, phenyl. Multicyclic rings include, but are not limited to, naphthyl and bicyclic rings wherein phenyl is fused to a C₁₋₇cycloalkyl or C₅₋₇cycloalkenyl ring. Aryl groups may be optionally substituted with one or more substituents as defined herein. Bonding can be through any of the carbon atoms of any ring.

‘H’ refers to hydrogen.

“Halo” or “halogen” refers to fluoro, chloro, bromo or iodo, unless otherwise noted.

“Heterocycle” or “heterocyclyl” refers to a saturated, partially unsaturated or aromatic ring moiety having at least one ring heteroatom and at least one ring carbon atom. In one embodiment, the heteroatom is oxygen, sulfur, or nitrogen. A heterocycle containing more than one heteroatom may contain different heteroatoms. Heterocyclyl moieties include both monocyclic and multicyclic (e.g., bicyclic) ring moieties. Bicyclic ring moieties include fused, spirocycle and bridged bicyclic rings and may comprise one or more heteroatoms in either of the rings. The ring attached to the remainder of the molecule may or may not contain a heteroatom. Either ring of a bicyclic heterocycle may be saturated, partially unsaturated or aromatic. The heterocycle may be attached to the rest of the molecule via a ring carbon atom, a ring oxygen atom or a ring nitrogen atom. Non-limiting examples of heterocycles are described below.

In one embodiment, partially unsaturated and aromatic 4-7 membered monocyclic heterocyclyl moieties include, but are not limited to, 2,3-dihydro-1,4-dioxinyl, dihydropyranyl, dihydropyrazinyl, dihydropyridazinyl, dihydropyridinyl, dihydropyrimidinyl, dihydrotriazolyl, furanyl, imidazolyl, isothiazolyl, isoxazolyl, oxadiazolyl, oxazolyl, oxoimidazolidinyl, pyranyl, pyrazinyl, pyrazolyl, pyridazinyl, pyridinyl, pyrimidinyl, pyrrolyl, tetrahydropyrazinyl, tetrahydropyridazinyl, tetrahydropyridinyl, tetrahydropyrimidinyl, tetrazolyl, thiadiazolyl, thiazolyl, thienyl, thiophenyl, and triazolyl.

In one embodiment, saturated 4-7 membered monocyclic heterocyclyl moieties include, but are not limited to, azetidinyl, 1,4-dioxanyl, hexahydroazepinyl, morpholinyl, 1,4-oxazepanyl, oxazolidinyl, oxetanyl, piperazinyl, piperidinyl, pyridin-2-onyl, pyrrolidinyl, tetrahydrofuranyl, tetrahydropyranyl, thiomorpholinyl, tetrahydrothienyl, and tetrahydrothiophenyl. In one embodiment, a saturated 4-7 membered monocyclic heterocyclyl is azetidinyl.

Heterocyclic groups may be optionally substituted with one or more substituents as defined herein,

“Optionally substituted” refers to “unsubstituted or substituted,” and therefore, the generic structural formulas described herein encompass compounds containing the specified optional substituent(s) as well as compounds that do not contain the optional substituent(s). Each substituent is independently defined each time it occurs within the generic structural formula definitions.

“Celite®” (Fluka) diatomite is diatomaceous earth and can be referred to as “celite”.

### Polymorphism

A compound disclosed herein, including a salt, solvate or hydrate thereof, may exist in crystalline form, non-crystalline form, or a mixture thereof. A compound or a salt or solvate thereof may also exhibit polymorphism, i.e. the capacity of occurring in different crystalline forms. These different crystalline forms are typically known as “polymorphs”. Polymorphs have the same chemical composition but differ in packing, geometrical arrangement, and other descriptive properties of crystalline solid state. Polymorphs, therefore, may have different physical properties such as shape, density, hardness, deformability, stability, and dissolution properties. Polymorphs typically exhibit different melting points. IR spectra, and X-ray powder diffraction patterns, all of which may be used for identification. One of ordinary skill in the art will appreciate that different polymorphs may be produced, for example, by changing or adjusting the conditions used in crystallizing/recrystallizing a compound disclosed herein.

### Optical Isomers-Diastereomers-Geometric Isomers-Tautomers

Included herein are various isomers of the compounds disclosed herein. The term “isomers” refers to compounds that have the same composition and molecular weight but differ in physical and/or chemical properties. The structural difference may be in constitution (geometric isomers) or in the ability to rotate the plane of polarized light (stereoisomers).

Regarding stereoisomers, a compound disclosed herein may have one or more asymmetric carbon atom and may occur as mixtures (such as a racemic mixture) or as individual enantiomers or diastereomers. All such isomeric forms are included herein, including mixtures thereof. If a compound disclosed herein contains a double bond, the substituent may be in the E or Z configuration. If a compound disclosed herein contains a disubstituted cycloalkyl, the cycloalkyl substituent may have a cis- or trans-configuration. All tautomeric forms are also intended to be included.

Any asymmetric atom (e.g., carbon) of a compound disclosed herein, can be present in racemic mixture or enantiomerically enriched, for example the (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

A compound disclosed herein, can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated based on the physicochemical differences of the constituents, into the pure or substantially pure geometric or optical isomers, diastereomers, racemates, for example, by chromatography and/or fractional crystallization.

Any resulting racemates of the final compounds of the examples or intermediates can be resolved into the optical antipodes by known methods, e.g., by separation of the diastereomeric salts thereof, obtained with an optically active acid or base, and liberating the optically active acidic or basic compound. A basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds can also be resolved by chiral chromatography, e.g., high pressure liquid chromatography (HPLC) using a chiral adsorbent.

Some of the compounds described herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are included within the scope of the present invention.

### Isotopic Variations

Compounds disclosed herein, include unlabeled forms, as well as isotopically labeled forms. Isotopically labeled compounds have structures depicted by the formulas given herein except that one or more atoms are replaced by an atom having a selected atomic mass or mass number. Examples of isotopes that can be incorporated into compounds disclosed herein include isotopes of hydrogen, carbon, nitrogen, oxygen, phosphorous, sulfur, fluorine, iodine and chlorine, such as ²H (i.e., Deuterium or “D”), ³H, ¹¹C, ¹³C, ¹⁴C, ¹³N, ¹⁵N, ¹⁵O, ¹⁷O, ¹⁸O ³²P, ³⁵S, ¹⁸F, ¹²³I, ¹²⁵I and ³⁶Cl. The invention includes various isotopically labeled compounds as defined herein, for example those into which radioactive isotopes, such as ³H and ¹⁴C, or those into which non-radioactive isotopes, such as ²H and ¹³C are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Substitution with positron emitting isotopes, such as ¹¹C, ¹⁸F, ¹⁵O and ¹³N, may be particularly desirable for PET or SPECT studies. Isotopically-labeled compounds disclosed herein, can generally be prepared by conventional techniques known to those skilled in the art. Furthermore, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index.

### Pharmaceutically Acceptable Salts

The term “pharmaceutically acceptable salt” refers to a salt prepared from a pharmaceutically acceptable non-toxic base or acid, including inorganic or organic base and inorganic or organic acid. Salts derived from inorganic bases include aluminum, ammonium, calcium, copper, ferric, ferrous, lithium, magnesium, manganic salts, manganous, potassium, sodium, zinc, and the like. Particular embodiments include ammonium, calcium, magnesium, potassium, and sodium salts. Salts in the solid form may exist in more than one crystal structure and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When a compound disclosed herein is basic, a salt may be prepared from a pharmaceutically acceptable non-toxic acid, including an inorganic and organic acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, trifluoroacetic acid (TFA) and the like. Particular embodiments include the citric, hydrobromic, hydrochloric, maleic, phosphoric, sulfuric, fumaric, tartaric and trifluoroacetic acids.

### Methods of Use

Compounds disclosed herein can inhibit activity of haematopoietic progenitor kinase 1 (HPK1). For example, the compounds disclosed herein can potentially be used to inhibit activity of HPK1 in cell or in an individual in need of modulation of the enzyme by administering an effective amount of a compound.

Also disclosed herein are methods of treating diseases associated with activity or expression, including abnormal activity and/or overexpression, of HPK1 in an individual (e.g., patient) by administering to the individual in need of such treatment an effective amount or dose of a compound disclosed herein or a pharmaceutical composition thereof. Example diseases can include any disease, disorder or condition that may be directly or indirectly linked to expression or activity of the HPK1 enzyme, such as over expression or abnormal activity. An HPK1-associated disease can also include any disease, disorder or condition that may be prevented, ameliorated, or cured by modulating enzyme activity.

Examples of HPK1-associated diseases include cancer, metastasis, inflammation and auto-immune pathogenesis. Example cancers potentially treatable by the methods herein include cancer of the colon, pancreas, breast, prostate, lung, brain, ovary, cervix, testes, renal, head and neck, lymphoma, leukemia, melanoma, and the like. In some embodiments, the cancer is selected from liposarcoma, neuroblastoma, glioblastoma, bladder cancer, adrenocortical cancer, multiple myeloma, colorectal cancer, non-small cell lung cancer, oropharyngeal cancer, penis cancer, anal cancer, thyroid cancer, vaginal cancer, gastric cancer, rectal cancer, thyroid cancer, Hodgkin lymphoma and diffuse large B-cell lymphoma. Another aspect of the invention relates to a method of inducing cell cycle arrest, apoptosis in tumor cells, and/or enhanced tumor-specific T cell immunity. The method comprises contacting the cells with an effective amount of a compound of Formula (I). In another embodiment, the present invention relates to a compound of Formula (I) or a pharmaceutical composition comprising a compound of the present invention and a pharmaceutically acceptable carrier used for the treatment of cancers including, but not limited to, liposarcoma, neuroblastoma, glioblastoma, bladder cancer, adrenocortical cancer, multiple myeloma, colorectal cancer, non-small cell lung cancer, oropharyngeal cancer, penis cancer, anal cancer, thyroid cancer, vaginal cancer, gastric cancer, rectal cancer, thyroid cancer. Hodgkin lymphoma and diffuse large B-cell lymphoma. In some embodiments, administration of a compound of Formula (I) or a pharmaceutical composition comprising a compound of the present invention and a pharmaceutically acceptable carrier induces a change in the cell cycle or cell viability

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the HPK1 enzyme with a compound disclosed herein includes the administration of a compound of the present invention to an individual or patient, such as a human, as well as, for example, introducing a compound of the invention into a sample containing a cellular or purified preparation containing the HPK1 enzyme.

A subject administered with a compound disclosed herein, or a pharmaceutically acceptable salt, solvate or hydrate thereof, is generally a mammal, such as a human being, male or female. A subject also refers to cows, sheep, goats, horses, dogs, cats, rabbits, rats, mice, fish, and birds. In one embodiment, the subject is a human.

As used herein, the terms “treatment” and “treating” refer to all processes wherein there may be a slowing, interrupting, arresting, controlling, or stopping of the progression of a disease or disorder that may be associated with HPK1 enzyme activity. The terms do not necessarily indicate a total elimination of all disease or disorder symptoms. The terms also include the potential prophylactic therapy of the mentioned conditions, particularly in a subject that is predisposed to such disease or disorder.

The terms “administration of” and or “administering a” compound should be understood to include providing a compound described herein, or a pharmaceutically acceptable salt, solvate or hydrate thereof, and compositions of the foregoing to a subject.

The amount of a compound administered to a subject is an amount sufficient to inhibit HPK1 enzyme activity in the subject. In an embodiment, the amount of a compound can be an “effective amount”, wherein the subject compound is administered in an amount that will elicit a biological or medical response of a tissue, system, animal or human that is being sought by a researcher, veterinarian, medical doctor or other clinician. An effective amount does not necessarily include considerations of toxicity and safety related to the administration of a compound. It is recognized that one skilled in the art may affect physiological disorders associated with an HPK1 enzyme activity by treating a subject presently afflicted with the disorders, or by prophylactically treating a subject likely to be afflicted with the disorders, with an effective amount of a compound disclosed herein, or a pharmaceutically acceptable salt, solvate or hydrate thereof.

An effective amount of a compound will vary with the particular compound chosen (e.g. considering the potency, efficacy, and/or half-life of the compound); the route of administration chosen; the condition being treated; the severity of the condition being treated; the age, size, weight, and physical condition of the subject being treated; the medical history of the subject being treated; the duration of the treatment; the nature of a concurrent therapy; the desired therapeutic effect; and like factors and can be routinely determined by the skilled artisan.

The compounds disclosed herein may be administered by any suitable route including oral and parenteral administration. Parenteral administration is typically by injection or infusion and includes intravenous, intramuscular, and subcutaneous injection or infusion.

The compounds disclosed herein may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound disclosed herein depend on the pharmacokinetic properties of that compound, such as absorption, distribution and half-life which can be determined by a skilled artisan. In addition, suitable dosing regimens, including the duration such regimens are administered, for a compound disclosed herein depend on the disease or condition being treated, the severity of the disease or condition, the age and physical condition of the subject being treated, the medical history of the subject being treated, the nature of concurrent therapy, the desired therapeutic effect, and like factors within the knowledge and expertise of the skilled artisan. It will be further understood by such skilled artisans that suitable dosing regimens may require adjustment given an individual subject's response to the dosing regimen or over time as the individual subject needs change. Typical daily dosages may vary depending upon the particular route of administration chosen. Typical daily dosages for oral administration, to a human weighing approximately 70 kg would range from about 0.1 mg to about 2 grams, or more specifically, 0.1 mg to 500 mg, or even more specifically, 0.2 mg to 100 mg, of a compound disclosed herein.

One embodiment of the present invention provides for a method of treating a disease or disorder associated with HPK1 enzyme activity comprising administration of an effective amount of a compound disclosed herein to a subject in need of treatment thereof. In one embodiment, the disease or disorder associated with an HPK1 enzyme is a cell proliferation disorder.

In one embodiment, disclosed herein is the use of a compound disclosed herein in a therapy. The compound may be useful in a method of inhibiting HPK1 enzyme activity in a subject, such as a mammal in need of such inhibition, comprising administering an effective amount of the compound to the subject.

In one embodiment, disclosed herein is a pharmaceutical composition comprising a compound disclosed herein, or a pharmaceutically acceptable salt, solvate or hydrate thereof, for use in potential treatment of a disorder or disease related to HPK1 enzyme activity.

### Compositions

The term “composition” as used herein is intended to encompass a dosage form comprising a specified compound in a specified amount, as well as any dosage form which results, directly or indirectly, from combination of a specified compound in a specified amount. Such term is intended to encompass a dosage form comprising a compound disclosed herein, or a pharmaceutically acceptable salt, solvate or hydrate thereof, and one or more pharmaceutically acceptable carriers or excipients. Accordingly, the compositions of the present invention encompass any composition made by admixing a compound of the present invention and one or more pharmaceutically acceptable carrier or excipients. By “pharmaceutically acceptable” it is meant the carriers or excipients are compatible with the compound disclosed herein and with other ingredients of the composition.

In one embodiment, disclosed herein is a composition comprising a compound disclosed herein, or a pharmaceutically acceptable salt, solvate or hydrate thereof, and one or more pharmaceutically acceptable carriers or excipients. The composition may be prepared and packaged in bulk form wherein an effective amount of a compound of the invention can be extracted and then given to a subject, such as with powders or syrups. Alternatively, the composition may be prepared and packaged in unit dosage form wherein each physically discrete unit contains an effective amount of a compound disclosed herein. When prepared in unit dosage form, the composition of the invention typically contains from about 0.1 mg to 2 grams, or more specifically, 0.1 mg to 500 mg, or even more specifically, 0.2 mg to 100 mg, of a compound disclosed herein, or a pharmaceutically acceptable salt, solvate or hydrate thereof. A compound disclosed herein and a pharmaceutically acceptable carrier or excipient(s) will typically be formulated into a dosage form adapted for administration to a subject by a desired route of administration. For example, dosage forms include those adapted for (1) oral administration, such as tablets, capsules, caplets, pills, troches, powders, syrups, elixirs, suspensions, solutions, emulsions, sachets, and cachets; and (2) parenteral administration, such as sterile solutions, suspensions, and powders for reconstitution. Suitable pharmaceutically acceptable carriers or excipients will vary depending upon the particular dosage form chosen. In addition, suitable pharmaceutically acceptable carriers or excipients may be chosen for a particular function that they may serve in the composition. For example, certain pharmaceutically acceptable carriers or excipients may be chosen for their ability to facilitate the production of uniform dosage forms. Certain pharmaceutically acceptable carriers or excipients may be chosen for their ability to facilitate the production of stable dosage forms. Certain pharmaceutically acceptable carriers or excipients may be chosen for their ability to facilitate the carrying or transporting of a compound disclosed herein, once administered to the subject, from one organ or portion of the body to another organ or another portion of the body. Certain pharmaceutically acceptable carriers or excipients may be chosen for their ability to enhance patient compliance.

Suitable pharmaceutically acceptable excipients include the following types of excipients: diluents, lubricants, binders, disintegrants, fillers, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents.

A skilled artisan possesses the knowledge and skill in the art to select suitable pharmaceutically acceptable carriers and excipients in appropriate amounts for the use in the invention. In addition, there are a number of resources available to the skilled artisan, which describe pharmaceutically acceptable carriers and excipients and may be useful in selecting suitable pharmaceutically acceptable carriers and excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The compositions of the invention are prepared using techniques and methods known to those skilled in the art. Some methods commonly used in the art are described in Remington's Pharmaceutical Sciences (Mack Publishing Company).

In one embodiment, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives, (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch) gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as, for example, by coating or embedding particulate material in polymers, wax, or the like.

The compounds disclosed herein may also be coupled with soluble polymers as targetable drug carriers. Such polymers can include polyvinylpyrrolidone, pyrancopolymer, polyhydroxypropylmethacrylamidephenol, polyhydroxyethylaspartamidephenol, or polyethyleneoxidepolylysine substituted with palmitoyl residues. Furthermore, the compounds of the invention may be coupled to a class of biodegradable polymers useful in achieving controlled release of a drug, for example polylactic acid, polyepsilon caprolactone, polyhydroxy butyric acid, polyorthoesters, polyacetals, polydihydropyrans, polycyanacrylates and cross-linked or amphipathic block copolymers of hydrogels.

In one embodiment, the invention is directed to a liquid oral dosage form. Oral liquids such as solution, syrups and elixirs can be prepared in dosage unit form so that a given quantity contains a predetermined amount of a compound disclosed herein. Syrups can be prepared by dissolving the compound of the invention in a suitably flavored aqueous solution; while elixirs are prepared using a non-toxic alcoholic vehicle. Suspensions can be formulated by dispersing a compound disclosed herein in a non-toxic vehicle. Solubilizers and emulsifiers such as ethoxylated isostearyl alcohols and polyoxy ethylene sorbitol ethers, preservatives, flavor additives such as peppermint oil or other natural sweeteners or saccharin or other artificial sweeteners and the like can also be added.

In one embodiment, the invention is directed to compositions for parenteral administration. Compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

### Combinations

A compound disclosed herein may be used in combination with one or more other active agents, including but not limited to, other anti-cancer agents, that are used in the prevention, treatment, control, amelioration, or reduction of risk of a particular disease or condition (e.g., cell proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present invention.

When a compound disclosed herein is used contemporaneously with one or more other active agents, a composition containing such other active agents in addition to the compound disclosed herein is contemplated. Accordingly, the compositions of the present invention include those that also contain one or more other active ingredients, in addition to a compound disclosed herein. A compound disclosed herein may be administered either simultaneously with, or before or after, one or more other therapeutic agent(s). A compound disclosed herein may be administered separately, by the same or different route of administration, or together in the same pharmaceutical composition as the other agent(s).

Products provided as a combined preparation include a composition comprising a compound disclosed herein and one or more other active agent(s) together in the same pharmaceutical composition, or a compound disclosed herein, and one or more other therapeutic agent(s) in separate form, e.g. in the form of a kit.

The weight ratio of a compound disclosed herein to a second active agent may be varied and will depend upon the effective dose of each agent. Generally, an effective dose of each will be used. Thus, for example, when a compound disclosed herein is combined with another agent, the weight ratio of the compound disclosed herein to the other agent will generally range from about 1000:1 to about 1:1000, such as about 200:1 to about 1:200. Combinations of a compound disclosed herein, and other active agents will generally also be within the aforementioned range, but in each case, an effective dose of each active agent should be used. In such combinations, the compound disclosed herein, and other active agents may be administered separately or in conjunction. In addition, the administration of one element may be prior to, concurrent to, or subsequent to the administration of other agent(s).

In one embodiment, the invention provides a composition comprising a compound disclosed herein, and at least one other therapeutic agent as a combined preparation for simultaneous, separate or sequential use in therapy. In one embodiment, the therapy is the treatment of a disease or disorder associated with HPK1 enzyme activity.

In one embodiment, the invention provides a kit comprising two or more separate pharmaceutical compositions, at least one of which contains a compound disclosed herein. In one embodiment, the kit comprises means for separately retaining said compositions, such as a container, divided bottle, or divided foil packet. An example of such a kit is a blister pack, as typically used for the packaging of tablets, capsules and the like.

A kit disclosed herein may be used for administering different dosage forms, for example, oral and parenteral, for administering the separate compositions at different dosage intervals, or for titrating the separate compositions against one another. To assist with compliance, a kit of the invention typically comprises directions for administration.

Disclosed herein is a use of a compound disclosed herein, for treating a disease or disorder associated with HPK1 enzyme activity, wherein the medicament is prepared for administration with another active agent. The invention also provides the use of another active agent for treating a disease or disorder associated with an HPK1 enzyme, wherein the medicament is administered with a compound disclosed herein.

The invention also provides the use of a compound disclosed herein for treating a disease or disorder associated with HPK1 enzyme activity, wherein the patient has previously (e.g. within 24 hours) been treated with another active agent. The invention also provides the use of another therapeutic agent for treating a disease or disorder associated with HPK1 enzyme activity, wherein the patient has previously (e.g. within 24 hours) been treated with a compound disclosed herein. The second agent may be applied a week, several weeks, a month, or several months after the administration of a compound disclosed herein.

In one embodiment, the other active agent is selected from the group consisting of vascular endothelial growth factor (VEGF) receptor inhibitors, topoisomerase II inhibitors, smoothen inhibitors, alkylating agents, anti-tumor antibiotics, anti-metabolites, retinoids, immunomodulatory agents including but not limited to anti-cancer vaccines, CTLA-4, LAG-3 and PD-1 antagonists.

Examples of vascular endothelial growth factor (VEGF) receptor inhibitors include, but are not limited to, bevacizumab (sold under the trademark AVASTIN by Genentech/Roche), axitinib, (N-methyl-2-[[3-[([pound])-2-pyridin-2-ylethenyl]-1H-indazol-6-yl]sulfanyl]benzamide, also known as AG013736, and described in PCT Publication No. WO 01/002369), Brivanib Alaninate ((S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinyimethyj)amino]-3-pyfidinecarboxamide, and described in PCT Publication No. WO 02/068470), pasireotide (also known as SO 230, and described in PCT Publication No. WO 02/010192), and sorafenib (sold under the tradename NEXAVAR).

Examples of topoisomerase II inhibitors, include but are not limited to, etoposide (also known as VP-16 and Etoposide phosphate, sold under the tradenames TOPOSAR, VEPESID and ETOPOPHOS), and teniposide (also known as VM-26, sold under the tradename VUMON).

Examples of alkylating agents, include but are not limited to, 5-azacytidine (sold under the trade name VIDAZA), decitabine (sold under the trade name of DECOGEN), temozolomide (sold under the trade names TEMODAR and TEMODAL by Schering-Plough/Merck), dactinomycin (also known as actinomycin-D and sold under the tradename COSMEGEN), melphalan (also known as L-PAM. L-sarcolysin, and phenylalanine mustard, sold under the tradename ALKERAN), altretamine (also known as hexamethylmelamine (HMM), sold under the tradename HEXALEN), carmustine (sold under the tradename BCNU), bendamustine (sold under the tradename TREANDA), busulfan (sold under the tradenames BUSULFEX and MYLERAN), carboplatin (sold under the tradename PARAPLATIN), lomustine (also known as CCNU, sold under the tradename CeeNU), cisplatin (also known as CDDP, sold under the tradenames PLATINOL and PLATINOL-AQ), chlorambucil (sold under the tradename LEUKERAN), cyclophosphamide (sold under the tradenames CYTOXAN and NEOSAR), dacarbazine (also known as DTIC, DIC and imidazole carboxamide, sold under the tradename DTIC-DOME), altretamine (also known as hexamethylmelamine (HMM) sold under the tradename HEXALEN), ifosfamide (sold under the tradename IFEX), procarbazine (sold under the tradename MATULANE), mechlorethamine (also known as nitrogen mustard, mustine and mechloroethamine hydrochloride, sold under the tradename MUSTARGEN), streptozocin (sold under the tradename ZANOSAR), thiotepa (also known as thiophosphoamide, TESPA and TSPA, and sold under the tradename THIOPLEX).

Examples of anti-tumor antibiotics include, but are not limited to, doxorubicin (sold under the tradenames ADRIAMYCIN and RUBEX), bleomycin (sold under the tradename LENOXANE), daunorubicin (also known as daunorubicin hydrochloride, daunomycin, and rubidomycin hydrochloride, sold under the tradename CERUBIDINE), daunorubicin liposomal (daunorubicin citrate liposome, sold under the tradename DAUNOXOME), mitoxantrone (also known as DHAD, sold under the tradename NOVANTRONE), epirubicin (sold under the tradename ELLENCE), idarubicin (sold under the tradenames IDAMYCIN, IDAMYCIN PFS), and mitomycn C (sold under the tradename MUTAMYCIN).

Examples of anti-metabolites include, but are not limited to, claribine (2-chlorodeoxy-adenosine, sold under the tradename LEUSTATIN), 5-fluorouracil (sold under the tradename ADRUCIL), 6-thioguanine (sold under the tradename PURINETHOL), pemetrexed (sold under the tradename ALIMTA), cytarabine (also known as arabinosylcytosine (Ara-C), sold under the tradename CYTOSAR-U), cytarabine liposomal (also known as Liposomal Ara-C, sold under the tradename DEPOCYT), decitabine (sold under the tradename DACOGEN), hydroxyurea (sold under the tradenames HYDREA, DROXIA and MYLOCEL), fludarabine (sold under the tradename FLUDARA), floxuridine (sold under the tradename FUDR), cladribine (also known as 2-chlorodeoxyadenosine (2-CdA) sold under the tradename LEUSTATIN), methotrexate (also known as amethopterin, methotrexate sodium (MTX), sold under the tradenames RHEUMATREX and TREXALL), and pentostatin (sold under the tradename NIPENT).

Examples of retinoids include, but are not limited to, alitretinoin (sold under the tradename PANRETIN), tretinoin (all-trans retinoic acid, also known as ATRA, sold under the tradename VESANOID), Isotretinoin (13-c/s-retinoic acid, sold under the tradenames ACCUTANE, AMNESTEEM, CLARAVIS, CLARUS, DECUTAN, ISOTANE, IZOTECH, ORATANE, ISOTRET, and SOTRET), and bexarotene (sold under the tradename TARGRETIN).

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in any of the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments. Examples of PD-1 antagonists include, but are not limited to, pembrolizumab (sold under the tradename KEYTRUDA) and nivolumab (sold under the tradename OPDIVO).

Examples of mAbs that bind to human PD-1, and useful in the treatment method, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358.

Examples of mAbs that bind to human PD-L1, and useful in the treatment method, medicaments and uses of the present invention, are described in WO2013/019906. WO2010/077634 A1 and U.S. Pat. No. 8,383,796. Specific anti-human PD-L1 mAbs useful as the PD-1 antagonist in the treatment method, medicaments and uses of the present invention include MPDL3280A. BMS-936559, MED14736, MSB0010718C and an antibody which comprises the heavy chain and light chain variable regions of SEQ ID NO:24 and SEQ ID NO:21, respectively, of WO2013/019906.

Other PD-1 antagonists useful in any of the treatment method, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

Examples of other cytotoxic agents include, but are not limited to, arsenic trioxide (sold under the tradename TRISENOX), asparaginase (also known as L-asparaginase, and Erwinia L-asparaginase, sold under the tradenames ELSPAR and KIDROLASE).

### Experimental Procedures

The following examples are intended to be illustrative only and not limiting in any way. Abbreviations not indicated below have their meanings as conventionally used in the art unless specifically stated otherwise.

| | |
|---|---|
| ACN or MeCN | acetonitrile |
| AcOH | acetic acid |
| t-AmOH or | tert-Amyl alcohol |
| tert-AmOH | |
| BrettPhos-Pd- | [(2-di-cyclohexylphosphino-3,6-dimethoxy-2′,4′,6′- |
| G3 | triisopropyl-1,1′-bipheny1)-2-(2′-amino-1,1′- |
| | biphenyl)]palladium(II) methanesulfonate |
| | methanesulfonate |
| t-BuBrettPhos | 2-(di-tert-butylphosphino)-2′,4′,6′- triisopropyl-3,6- |
| | dimethoxy-1,1′-biphenyl |
| t-BuBrettPhos- | [(2-di-tert-butylphosphino-3,6-dimethoxy-2′,4′,6′- |
| Pd-G3 | triisopropyl-1,1′-biphenyl)-2-(2′-amino-1,1′- |
| | biphenyl)]palladium(II) methanesulfonate |
| BisPin | bis(pinacolato)diboron |
| Boc | tert-butoxycarbamate |
| Boc₂O | di-tert-butyl dicarbonate |
| DCM | dichloromethane |
| DIEA | diisopropylethylamine |
| DMA | dimethylacetamide |
| DMF | dimethylformamide |
| DMSO | dimethylsulfoxide |
| EI | electron ionization |
| EtOAc | ethyl acetate |
| EtOH | ethanol |
| ¹H NMR | proton nuclear magnetic resonance (data) |
| HPLC | high performance liquid chromatography |
| KOAc | potassium acetate |
| K₃PO₄ | potassium phosphate |
| LC/MS | liquid chromatography coupled to mass spectrometer |
| LiHMDS | lithium hexamethyldisilazide or lithium |
| | bis(trimethylsilyl)amide |
| MeCN | acetonitrile |
| MeOH | methanol |
| MHz | megahertz |
| MS | mass spectrum (data) |
| NBS | N-bromosuccinimide |
| NCS | N-chlorosuccinimide |
| MTBE | methyl tert-butyl ether |
| N(Et)₃ | triethylamine |
| NMR | nuclear magnetic resonance (data) |
| Na₂SO₄ | sodium sulfate |
| NaBH(OAc)₃ | sodium triacetoxyborohydride |
| NaOAc | sodium acetate |
| PdCl₂(dppf) | 1,1′-bis(diphenylphosphino)ferrocene-palladium(II)di- |
| | chloride |
| PdCl₂(dtbpf) | [1,1′-bis(di-tert-butylphosphino)ferrocene]di- |
| | chloropalladium(II) |
| Pd(PPh₃)₄ | tetrakis(triphenylphosphine)palladium(0) |
| Pd[(t-Bu)₃P]₂ | bis(tri-tert-butylphosphine)palladium(0) |
| Pd₂(dba)₃ | tris(dibenzylideneacetone)dipalladium(0) |
| Pd(PPh₃)₂Cl₂ | bis(triphenylphosphine)palladium(II) dichloride |
| PMB | para-methoxybenzyl |
| PPA | polyphosphoric acid |
| i-Pr₂Net | N,N-diisopropylethylamine |
| RT | room temperature |
| TFA | trifluoroacetic acid |
| THF | tetrahydrofuran |
| TMSCl | trimethylsilyl chloride |
| TLC | thin layer chromatography |
| Xphos | 2-dicyclohexylphosphino-2′,4′,6′-triisopropylbiphenyl |
| G2-XPhos | chloro(2-dicyclohexylphosphino-2′,4′,6′-triisopropyl- |
| Precatalyst | 1,1′-biphenyl)[2-(2′-amino-1,1′-biphenyl)]palladium(II) |

### General Synthetic Schemes

The compounds of formula (I) may be prepared by methods known in the art of organic synthesis as set forth in part by the following synthetic schemes and synthetic procedures and conditions for the illustrative intermediates and examples.

The compounds described herein may be made from commercially available starting materials or synthesized using known organic, inorganic, and/or enzymatic processes.

While the present invention has been described in conjunction with the specific examples set forth below, many alternatives, modifications, and variations thereof will be apparent to those of ordinary skill in the art. All such alternatives, modifications, and variations are intended to fall within the spirit and scope of the present invention.

### Methods of Synthesis

The compounds in the present invention can be prepared according to the following general schemes using appropriate materials and are further exemplified by the subsequent specific examples. The compounds illustrated in the examples are not to be construed as forming the only genus that is considered as the invention. The illustrative examples below, therefore, are not limited by the compounds listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of the instant invention herein above.

Those skilled in the art will readily understand that known variations of the conditions and processes of the following preparative procedures can be used to prepare these compounds. The invention will now be illustrated in the following non-limiting Examples in which, unless otherwise stated, all reactions were stirred (mechanically, stir bar/stir plate, or shaken) and conducted under an inert atmosphere of nitrogen or argon unless specifically stated otherwise. All temperatures are degrees Celsius (° C.) unless otherwise noted. Ambient temperature is 15-25° C. Most compounds were purified by reverse-phase preparative HPLC, MPLC on silica gel, recrystallization and/or trituration (suspension in a solvent followed by filtration of the solid). The course of the reactions was followed by thin layer chromatography (TLC) and/or LC/MS and/or NMR and reaction times are given for illustration only. All end products were analyzed by NMR and LC/MS. Intermediates were analyzed by NMR and/or TLC and/or LC/MS.

### General Synthetic Schemes

Several synthetic methods were employed to access the compounds described herein. Final compounds were evaluated for biological activity in the kinase activity assay in either the neutral form, as a TFA salt, or as a HCl salt, and were screened in the kinase activity assay as either the racemate or as resolved enantiomers and diastereomers. The chiral separations were conducted on either the final compounds or on a synthetic intermediate. Chiral separation conditions are noted where appropriate.

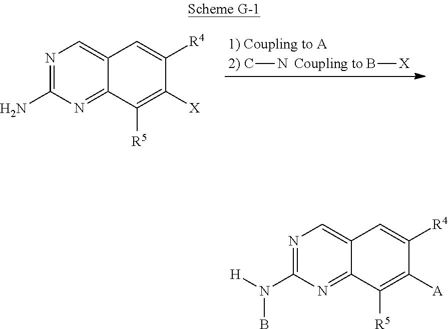

A general synthetic approach is outlined in Scheme G-1. An appropriately substituted 2-amino-7-bromo-quinazoline is used as a precursor, in which the A group is introduced at C(7) position of the quinazoline first. Next, the corresponding advanced intermediate is treated with an aryl halide or heteroaryl halide in the presence of Pd catalyst to install the B group.

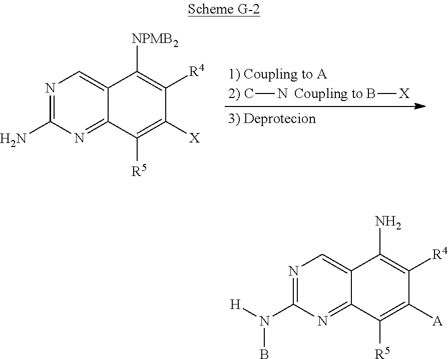

Compounds bearing an amine functional group at the 5-position of the quinazoline ring (R³═NH₂) are prepared as shown in Schemes G-2 and G-3. For these compounds, the synthetic sequence is modified in several ways, one shown in Scheme G-2. One modification is to prepare a substituted 2-amino-7-bromoquinazoline bearing an N(PMB)₂ group at the 5-position of the quinazoline. Similar to the methods described in Scheme G-1, introduction of the A group at the quinazoline 7-position by Pd-catalyzed C—C coupling then followed by C—N coupling with haloazoles to introduce the B group. This provides an advanced intermediate bearing the N(PMB)₂ group at the 5-position of the quinazoline; acid-mediated deprotection finally provides the final amine-bearing compounds with R³═NH₂.

### Synthesis of Common Intermediates

### Preparation of Intermediate I-1 (tert-Butyl 8-Methyl-7-(4,4,5,5-tetramethyl-1,3,2-dioxaborolan-2-yl)-2,3-dihydro-1H-pyrido[2,3-b][1,4]oxazine-1-carboxylate)

Intermediate I-1 was prepared from 4-methyl-3-nitropyridin-2-amine via the sequence outlined below.

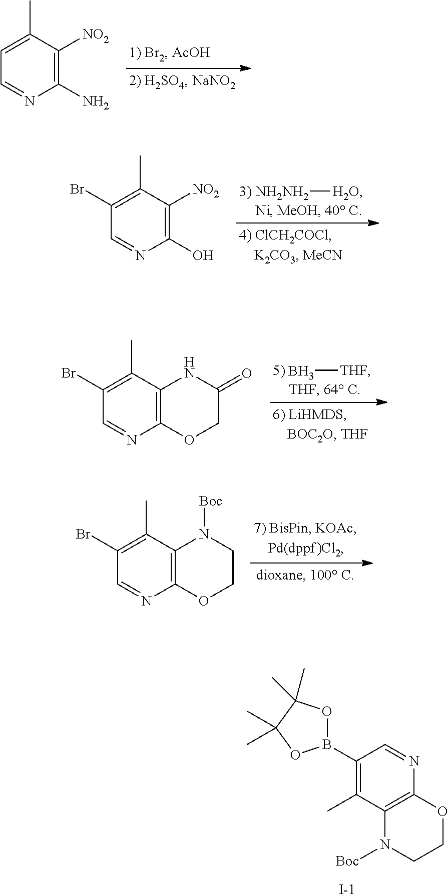

### Step 1. Synthesis of 5-Bromo-4-methyl-3-nitropyridin-2-amine

Two mixtures containing 4-methyl-3-nitropyridin-2-amine (400 g, 2.61 mol) in AcOH (2.8 L) were treated with NaOAc (82.0 g, 5.22 mol). Next, a solution of bromine (417 g, 2.61 mol) in AcOH (800 mL) was added dropwise at RT to each, and then the mixtures stirred for 2 h. The crude reaction mixtures were quenched with ice water (2 L), stirred for 30 min and finally combined into one. The combined mixture was filtered, and the filter cake washed with ice water (3×1 L) and dried under reduced pressure obtaining 5-bromo-4-methyl-3-nitropyridin-2-amine (1.0 kg) as a solid.

### Step 2. Synthesis of 5-Bromo-4-methyl-3-nitropyridin-2-ol

Five equal mixtures containing 5-bromo-4-methyl-3-nitropyridin-2-amine (200 g, 0.862 mol) in water (3.5 L) were treated each with H₂SO₄ (200 g, 2.04 mol) followed by NaNO₂ at 0° C. (137 g, 1.98 mol in 500 mL of water). The reactions were stirred at RT for 2 h, then at 100° C. for 4 h. The crude reaction mixtures were then combined and filtered. The filter cake was dried under reduced pressure obtaining 5-bromo-4-methyl-3-nitropyridin-2-ol (920 g) as a solid.

### Step 3. Synthesis of 3-Amino-5-bromo-4-methylpyridin-2-ol

Four equal mixtures containing 5-bromo-4-methyl-3-nitropyridin-2-ol (230 g, 0.987 mol) in MeOH (4.6 L) were treated with Raney-Ni (101 g, 1.18 mol). The mixtures were heated to 40° C. and treated with hydrazine hydrate (74.1 g, 1.48 mol) and the reaction mixtures stirred for 2 h at 40° C. The four mixtures were filtered and the filtrates combined into one. The combined filtrates were concentrated to remove MeOH, obtaining 3-amino-5-bromo-4-methylpyridin-2-ol (680 g) as a solid.

### Step 4. Synthesis of 7-Bromo-8-methyl-1H-pyrido[2,3-b][1,4]oxazin-2(3H)-one

Three equal mixtures containing 3-amino-5-bromo-4-methylpyridin-2-ol (227 g, 1.12 mol), K₂CO₃ (617 g, 4.47 mol) in MeCN (3.4 L) were cooled to 15° C. and each treated dropwise with chloroacetyl chloride (189 g, 1.67 mol). The mixtures were stirred at RT for 2 h, then at 40° C. for 6 h, and finally at 60° C. for 10 h. Each was then quenched with MeOH (100 mL), combined into one and filtered. The filted cake was washed with 2:1 THF/MeOH (3×1 L), and the filtrate concentrated in vacuo to obtain 7-bromo-8-methyl-1H-pyrido[2,3-b][1,4]oxazin-2(3H)-one (600 g) as a solid. ¹H NMR (400 MHz, CD₃OD) δ 7.78 (s, 1H), 4.68 (s, 2H), 3.35 (s, 1H), 2.38 (s, 3H).

### Step 5. Synthesis of 7-Bromo-8-methyl-2,3-dihydro-1H-pyrido[2,3-b][1,4]oxazine

Six equal mixtures of 7-bromo-8-methyl-1H-pyrido[2,3-b][1,4]oxazin-2(3H)-one (100 g, 411 mol) in THF (2 L) were treated dropwise at 0° C. with a 10 M solution of BH₃-Me₂S in THF (165 mL). The mixtures were stirred at 80° C. for 2 h, cooled to 0° C. and quenched with 500 mL of MeOH each. The six mixtures were stirred at 80° C. for 2 h, then combined into one and concentrated in vacuo, obtaining 7-bromo-8-methyl-2,3-dihydro-1H-pyrido[2,3-b][1,4]oxazine (400 g). ¹H NMR (400 MHz, CD₃OD) δ 7.50 (s, 1H), 4.31 (s, 2H), 3.43 (m, 2H), 2.23 (s, 3H).

### Step 6. Synthesis of tert-Butyl 7-Bromo-8-methyl-2,3-dihydro-1H-pyrido[2,3-b][1,4]oxazine-1-carboxylate

Two equal mixtures of 7-bromo-8-methyl-2,3-dihydro-1H-pyrido[2,3-b][1,4]oxazine (200 g, 873 mol) in THF (3 L) were treated with Boc₂O (229 g, 1.05 mol) and cooled to 0° C. To each were added a 1 M solution of LiHMDS in THF (1.05 L) and the reactions stirred at RT for 15 h. Each were quenched with MeOH (50 mL) and combined into one mixture which was concentrated in vacuo. The residue was then purified by chromatography (gradient of 40:1 to 3:1 petroleum ether/EtOAc) to give crude solid product. The material was then triturated with petroleum ether to obtain tert-butyl 7-bromo-8-methyl-2,3-dihydro-1H-pyrido[2,3-b][1,4]oxazine-1-carboxylate (186 g) as a solid. ¹H NMR (400 MHz, CD₃OD) δ 8.14 (s, 1H), 4.55 (m, 1H), 4.46 (m, 1H), 4.39 (m, 1H), 3.08 (m, 1H), 2.29 (s, 3H), 1.48 (s, 9H).

### Step 7. Synthesis of tert-Butyl 8-Methyl-7-(4,4,5,5-tetramethyl-1,3,2-dioxaborolan-2-yl)-2,3-dihydro-1H-pyrido[2,3-b][1,4]oxazine-1-carboxylate

A mixture of tert-butyl 7-bromo-8-methyl-2,3-dihydro-1H-pyrido[2,3-b][1,4]oxazine-1-carboxylate (186 g, 565 mol) in dioxane (1.86 L) was treated with bis(pinacolato)diboron (287 g, 1.13 mol), KOAc (166 g, 1.70 mol), and Pd(dppf)Cl₂ (41.3 g, 56.5 mmol). The mixture was purged and degassed with nitrogen three times, then stirred at 100° C. for 1 h. The reaction mixture was concentrated in vacuo. EtOAc (1 L) added and the mixture filtered. The EtOAc filtrate was treated with water (200 mL) and extracted with EtOAc (3×200 mL). The combined organic layers was washed with 100 mL of brine, dried (Na₂SO₄) and concentrated under reduced pressure. The residue was purified by chromatography on SiO₂ (gradient of 50:1 to 1:1 petroleum ether/EtOAc) to give crude product. The material was washed with petroleum ether to obtain tert-butyl 8-methyl-7-(4,4,5,5-tetramethyl-1,3,2-dioxaborolan-2-yl)-2,3-dihydro-1H-pyrido[2,3-b][1,4]oxazine-1-carboxylate (100 g) as a solid. ¹H NMR (400 MHz, CD₃OD) δ 8.37 (s, 1H), 4.53 (m, 1H), 4.46 (m, 1H), 4.34 (m, 1H), 3.08 (m, 1H), 2.40 (s, 3H), 1.47 (s, 9H), 1.33 (s, 12H). MS (EI) calc'd for C₁₉H₃₀BN₂O₅ [M+H]⁺, 377; found 377.

### Preparation of Intermediate I-2 (7-Bromoquinazolin-2-amine)

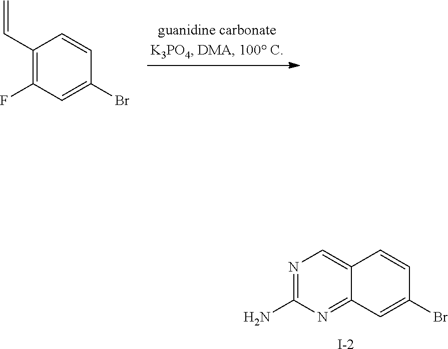

A 30-mL microwave vial was charged with guanidine carbonate (486 mg, 2.70 mmol), 4-bromo-2-fluorobenzaldehyde (548 mg, 2.70 mmol) and DMA (13 mL). The vial was capped and heated to 100° C. for 4 h. After 4 h, the reaction was cooled, poured into 50 mL of ice water, stirred for 15 min then filtered to give a 7-bromoquinazolin-2-amine (553 mg) as a powdery solid. MS (EI) calc'd for C₈H₇BrN₃ [M+H]⁺, 224, 226; found 224, 226.

### Preparation of Intermediate I-3 (7-Bromo-6-fluoro-N⁵,N⁵-bis(4-methoxybenzyl)quinazoline-2,5-diamine)

Intermediate I-3 was prepared from 1-bromo-2,3,5-trifluorobenzene as shown below.

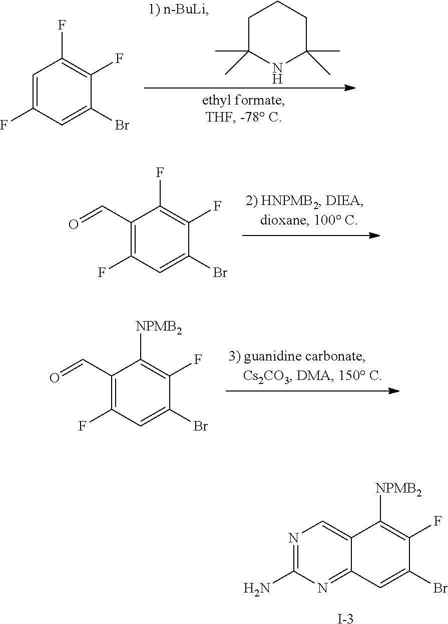

### Step 1. Synthesis of 4-Bromo-2,3,6-trifluorobenzaldehyde

A mixture of 2,2,6,6-tetramethylpiperidine (187 g, 1.33 mol) in THF (1.4 L) was cooled to −78° C. and treated dropwise with a 2.5 M solution of n-BuLi (400 mL, 1.00 mol) and stirred for 30 min. Next, 1-bromo-2,3,5-trifluorobenzene (140 g, 663 mmol) was added dropwise as a solution in THF (1.12 L) and the reaction mixture stirred for 1 h at −78° C. Next, a solution of ethyl formate (59 g, 796 mol) in THF (280 mL) was added dropwise and the reaction mixture stirred another 1 h. The mixture was finally quenched with the addition of aqueous NH₄Cl (2 L) and extracted with EtOAc (3 L, 2 L, 1 L). The combined organic layers were concentrated and the residue purified by chromatography on SiO₂ (100:1 to 100:5 petroleum ether/EtOAc) to provide 4-bromo-2,3,6-trifluorobenzaldehyde (42 g) as a solid. ¹H NMR (400 MHz, CDCl₃) δ 10.13 (s, 1H), 7.86-7.90 (m, 1H).

### Step 2. Synthesis of 2-(Bis(4-methoxy benzyl)amino)-4-bromo-3,6-difluorobenzaldehyde

A solution of 4-bromo-2,3,6-trifluorobenzaldehyde (50 g, 209 mmol), DIEA (55 g, 430 mmol), HNPMB₂ (63 g, 240 mmol) in dioxane (500 mL) was warmed to 100° C. and stirred for 6 h. The mixture was concentrated and the residue purified by chromatography on SiO₂ (100:1 to 100:5, petroleum ether/EtOAc) to provide 2-(bis(4-methoxybenzyl)amino)-4-bromo-3,6-difluorobenzaldehyde (90 g) as an oil. ¹HNMR (400 MHz, DMSO-d₆) δ 10.07 (s, 1H), 7.56-7.56 (m, 1H), 7.08-7.01 (m, 4H), 6.83-6.86 (m, 4H), 4.174 (s, 4H), 3.71 (s, 6H).

### Step 3. Synthesis of 7-Bromo-6-fluoro-N⁵,N⁵-bis(4-methoxybenzyl)quinazoline-2,5-diamine

A solution of 2-(bis(4-methoxybenzyl)amino)-4-bromo-3,6-difluorobenzaldehyde (90 g, 189 mmol), Cs₂CO₃ (122 g, 374 mmol), guanidine carbonate (44 g, 242 mmol) in DMA (360 mL) was warmed to 150° C. and stirred for 30 min. The reaction mixture was cooled to RT, diluted with water (2 L) and extracted with EtOAc (2 L, 1 L). The organic layer was concentrated and the residue purified by chromatography on SiO₂ (100:1 to 2.5:1 petroleum ether/EtOAc) to provide 7-bromo-6-fluoro-N⁵,N⁵-bis(4-methoxybenzyl)quinazoline-2,5-diamine (31 g) as a solid. ¹H NMR (400 MHz, DMSO-d₆) δ 9.24 (s, 1H), 7.46-7.47 (d, J=4 Hz, 1H), 7.13-7.15 (d, J=8 Hz, 4H), 6.93 (s, 2H), 6.80-6.82 (d. J=8 Hz, 4H), 4.21 (s, 4H), 3.68 (s, 6H). MS (EI) calc'd for C₂₄H₂₃BrFN₄O₂ [M+H]⁺, 497, 499; found 497, 499.

### Preparation of Intermediate I-4 (tert-Butyl 7-(2-Aminoquinazolin-7-yl)-8-methyl-2,3-dihydro-1H-pyrido[2,3-b][1,4]oxazine-1-carboxylate)

Intermediate I-4 was prepared via the coupling of intermediates I-1 and I-2.

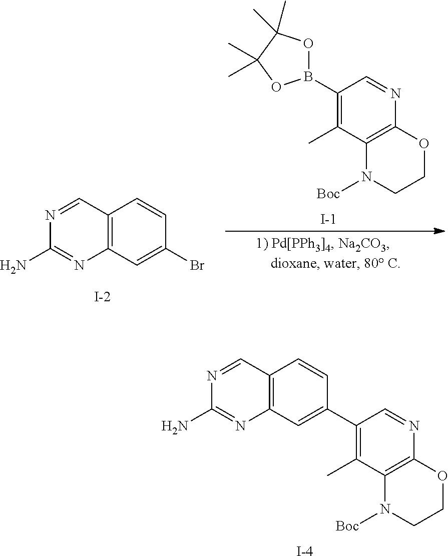

A mixture containing 7-bromoquinazolin-2-amine (I-2; 50 mg, 0.22 mmol), tert-butyl 8-methyl-7-(4,4,5,5-tetramethyl-1,3,2-dioxaborolan-2-yl)-2,3-dihydro-1H-pyrido[2,3-b][1,4]oxazine-1-carboxylate (Intermediate I-1; 100 mg, 0.27 mmol), PdCl₂(dppf) (16 mg, 0.022 mmol) and Na₂CO₃ (50 mg, 0.47 mmol) in dioxane (1 mL) and water (0.2 mL) was stirred overnight at 80° C. The mixture was diluted with DCM, washed with water. Organic layer was dried (Na₂SO₄) and concentrated. Chromatography on SiO₂ (gradient of 0-20% MeOH/DCM, 25 g silica gel) gave the desired product I-4 as a solid. MS (EI) calc'd for C₂₁H₂₄N₅O₃ [M+H]⁺, 416: found, 416.

### Preparation of Intermediate I-5 (tert-Butyl 7-(2-Amino-5-(bis(4-methoxybenzyl)amino)-6-fluoroquinazolin-7-yl)-8-methyl-2,3-dihydro-1H-pyrido[2,3-b][1,4]oxazine-1-carboxylate)

Intermediate I-5 was prepared via the coupling of intermediates I-1 and I-3.

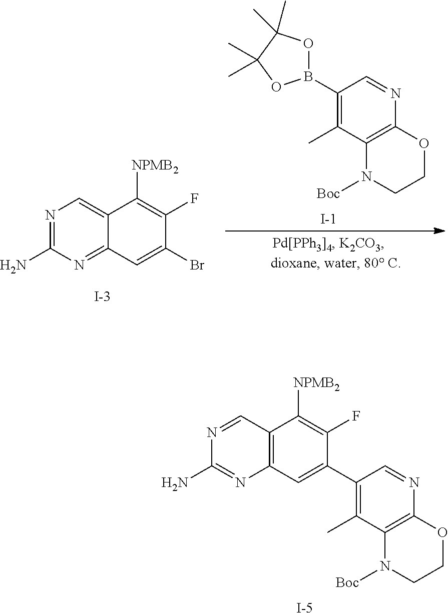

A mixture containing 7-bromo-6-fluoro-N⁵,N⁵-bis(4-methoxybenzyl)quinazoline-2,5-diamine (I-3) (5.0 g, 10 mmol), tert-butyl 8-methyl-7-(4,4,5,5-tetramethyl-1,3,2-dioxaborolan-2-yl)-2,3-dihydro-1H-pyrido[2,3-b][1,4]oxazine-1-carboxylate (I-1) (4.5 g, 12 mmol), Pd(PPh₃)₄ (1.16 g mg, 1.01 mmol), K₂CO₃ (2.8 g, 20 mmol) in dioxane (15 mL) and water (5 mL) was stirred overnight at 80° C. The reaction mixture filtered, and the residue washed with EtOAc.

The combined organic layers were concentrated under reduced pressure and purified by chromatography on SiO₂ (gradient of 0-20% MeOH/DCM, 120 g silica gel) to give the desired product, I-5, as an oil that solidified upon standing. ¹H NMR (500 MHz, CDCl₃) δ 9.53 (s, 1H), 7.92 (s, 1H), 7.24 (m, 1H), 7.17 (2 d, 4H), 6.79 (2 d, 4H), 5.25 (br, 2H), 4.61 (br, 1H), 4.54 (br, 1H), 4.39 (br, 1H), 4.28 (s, 4H), 3.77 (s, 6H), 3.17 (br, 1H), 2.02 (s, 3H), 1.26 (s, 9H); MS (EI) calc'd for C₃₇H₄₀FN₆O₅ [M+H]⁺, 667; found, 667.

### COMPOUND EXAMPLES OF TABLE 1

### Example 1A. Preparation of 1-1

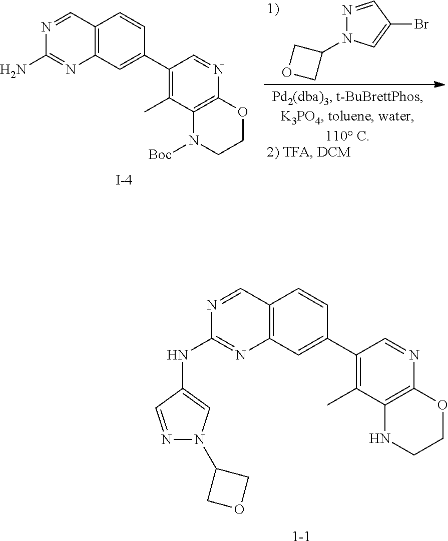

A mixture containing K₃PO₄ (40 mg, 0.19 mmol), Pd₂(dba)₃ (12 mg, 0.013 mmol), t-BuBrettPhos (12 mg, 0.025 mmol), 4-bromo-1-(oxetan-3-yl)-1H-pyrazole (25 mg, 0.12 mmol) and tert-butyl 7-(2-aminoquinazolin-7-yl)-8-methyl-2,3-dihydro-1H-pyrido[2,3-b][1,4]oxazine-1-carboxylate (I-4) (25 mg, 0.064 mmol) in toluene (1 mL) was treated with water (10 μl, 0.56 mmol) and deoxygenated by bubbling argon gas through the reaction mixture for 3 min. The mixture was stirred overnight at 110° C. The reaction was cooled, filtered and concentrated. The crude residue was dissolved in 1 mL DCM treated with 0.2 mL of TFA then aged for 3 h. After concentration the deprotected product was purified by reverse phase chromatography (gradient of 15-70% MeCN/water with 0.1% NH₄OH) to provide 1-1 as a solid.

¹H NMR (500 MHz, CD₃OD-d₄) δ 9.17 (s, 1H), 8.44 (s, 1H), 7.89 (d, J=8.2 Hz, 1H), 7.81 (s, 1H), 7.61 (d, J=6.2 Hz, 1H), 7.42 (s, 1H), 7.28 (d, J=8.2 Hz, 1H), 5.59 (p, J=6.9 Hz, 1H), 5.08 (d, J=6.8 Hz, 4H), 4.47-4.36 (m, 2H), 3.56-3.47 (m, 2H), 2.12 (s, 3H). MS (EI) calc'd for C₂₂H₂₂N₇O₂ [M+H]⁺, 416; found, 416.

### Example 1B. Preparation of 1-2

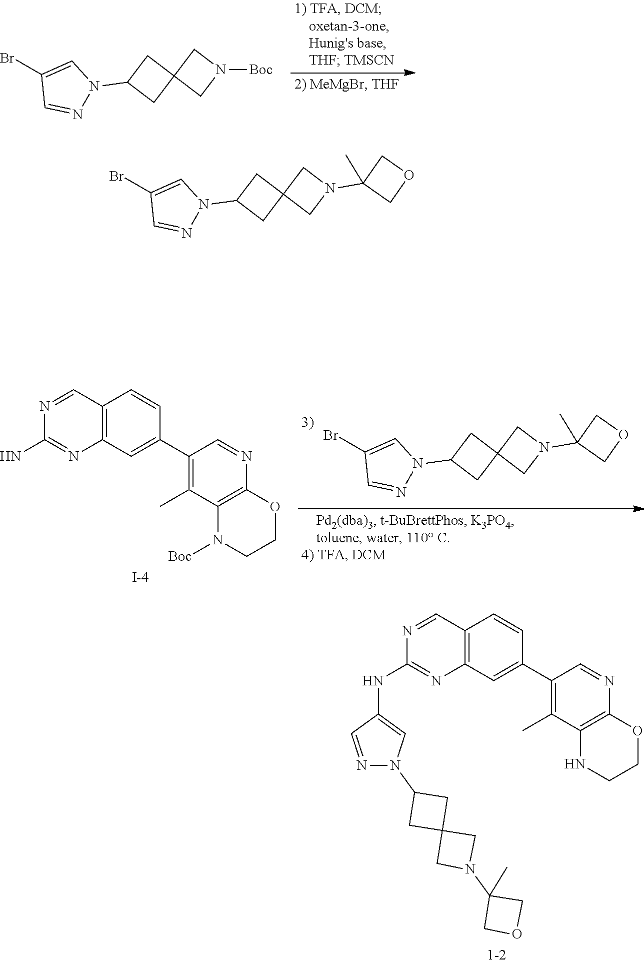

### Step 1. Preparation of 3-(6-(4-Bromo-1H-pyrazol-1-yl)-2-azaspiro[3,3]heptan-2-yl)oxetane-3-carbonitrile

A solution of tert-butyl 6-(4-bromo-1H-pyrazol-1-yl)-2-azaspiro[3.3]heptane-2-carboxylate (500 mg, 1.46 mmol) in a 3 mL of DCM and TFA (1 mL) was stirred for 4 h, then concentrated to a solid. The solid residue was dissolved in THF (3 mL), treated with Hunig's base (1.3 mL, 7.3 mmol) and oxetan-3-one (0.21 mL, 3.7 mmol). The reaction mixture was stirred at room temperature for 20 min, after which trimethylsilanecarbonitrile (0.22 mL, 1.8 mmol) and acetic acid (0.10 mL, 1.8 mmol) were added. The reaction mixture was heated to 70° C. and stirred for 5 hours. The crude reaction mixture was concentrated and purified by column chromatography (0-100% 3.1 EtOAc/EtOH in hexane) to afford 3-(6-(4-bromo-1H-pyrazol-1-yl)-2-azaspiro[3.3]heptan-2-yl)oxetane-3-carbonitrile. MS (EI) calc'd for C₁₃H₁₆BrN₄O [M+H]+, 323, 325; found, 323, 325.

### Step 2. Preparation of 6-(4-Bromo-1H-pyrazol-1-yl)-2-(3-methyloxetan-3-yl)-2-azaspiro[3.3]heptane

A 20-mL vial with 3-(6-(4-bromo-1H-pyrazol-1-yl)-2-azaspiro[3.3]heptan-2-yl)oxetane-3-carbonitrile (170 mg, 0.526 mmol) in THF (2.6 mL) was treated with 3.4 M ether solution of MeMgBr (0.77 mL, 2.6 mmol). The reaction was heated to 65° C. and 3 h, then quenched with 1 M NaOH. The aqueous phase was extracted with DCM, and the combined organic layers dried over Na₂SO₄, filtered, and concentrated. The crude residue was purified by column chromatography (0-100% EtOAc/hexanes) to give 6-(4-bromo-1H-pyrazol-1-yl)-2-(3-methyloxetan-3-yl)-2-azaspiro[3.3]heptane as a clear oil. ¹H NMR (500 MHz, CDCl₃) 7.50 (s, 1 H), 7.43 (s, 1H), 4.78 (m, 2H), 4.64 (m, 2H), 4.58 (m, 1H), 3.53 (s, 2H), 3.48 (s, 2H), 2.73 (m, 4H), 2.22 (s, 3H).

Steps 3 and 4. Preparation of 7-(8-Methyl-2,3-dihydro-1H-pyrido[2,3-b][1,4]oxazin-7-yl)-N-(1-(2-(3-methyloxetan-3-yl)-2-azaspiro[3.3]heptan-6-yl)-1H-pyrazol-4-yl)quinazolin-2-amine.

A mixture of 6-(4-bromo-1H-pyrazol-1-yl)-2-(3-methyloxetan-3-yl)-2-azaspiro[3.3]heptane (30 mg, 0.0% mmol) and tert-butyl 7-(2-aminoquinazolin-7-yl)-8-methyl-2,3-dihydro-1H-pyrido[2,3-b][1,4]oxazine-1-carboxylate (I-4) (25 mg, 0.064 mmol) in toluene (1 mL) was treated with Pd₂(dba)₃ (12 mg, 0.013 mmol), t-BuBrettPhos (12 mg, 0.025 mmol), K₃PO₄ (40 mg, 0.19 mmol) and water (0.010 mL) was deoxygenated by bubbling argon for 3 min and stirred overnight at 110° C. Filtered the mixture and concentrated. Residue was dissolved in 1 mL of DCM, treated with 0.2 mL of TFA, aged for 3 hours, then concentrated. Dissolved in 3 mL of MeOH and purified by reverse phase chromatography (gradient of 15-70% MeCN/water with 0.1% NH₄OH) to provide the desired product. ¹H NMR (500 MHz, CD₃OD) δ 9.16 (s, 1H),

8.31 (br, 1H), 7.88 (d, J=10 Hz, 1H), 7.72 (s, 1H), 7.60 (s, 1H), 7.41 (s, 1H), 7.27 (d, J=10 Hz, 1H), 4.80 (m, 1H), 4.73 (m, 2H), 4.41 (m, 2H), 4.35 (m, 2H), 3.59 (s, 2H), 3.51 (m, 4H), 2.72-2.77 (m, 4H), 2.11 (s, 3H), 1.34 (s, 3H); MS (EI) Calc'd for C₂₉H₃₃NO₂ [M+H]⁺, 525; found, 525.

### Example 1C. Preparation of 1-3

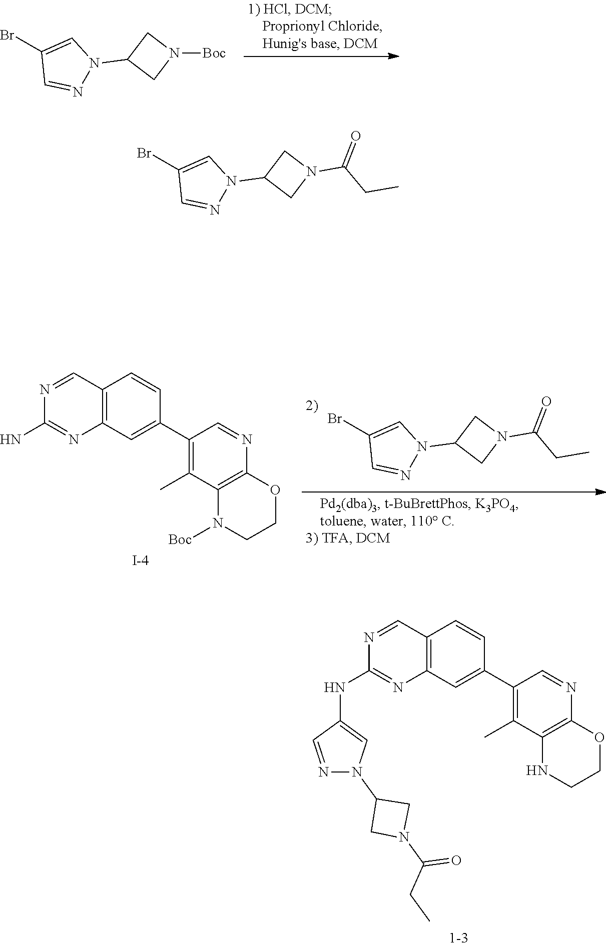

### Step 1. Preparation of 1-(3-(4-Bromo-1H-pyrazol-1-yl)azetidin-1-yl)propan-1-one

A mixture of tert-butyl 3-(4-bromo-1H-pyrazol-1-yl)azetidine-1-carboxylate (200 mg, 0.662 mmol) in DCM (3 mL) was treated with a 4 M dioxane solution of HCl (0.50 mL, 2.0 mmol). The mixture was aged for 6 hours, concentrated to dryness. It was redissolved in DCM (3 mL), treated with Hunig'sBase (0.30 mL, 1.7 mmol) followed by propionyl chloride (100 mg, 1.08 mmol). Stirred for 2 hours and concentrated. It was redissolved in 3 mL of MeOH and purified by reverse phase chromatography (gradient of 2-55% MeCN/water with 0.1% NH₄OH) to provide the desired intermediate. ¹H NMR (500 MHz, DMSO-d₆) δ 8.18 (s, 1H), 7.69 (s, 1H), 5.23 (m, 1H), 4.55 (s, 1H), 4.35 (m, 1H), 4.28 (m, 1H), 4.08 (m, 1H), 2.10 (q, J=10 Hz, 2H), 0.98 (t, J=10 Hz, 3H).

Steps 2 and 3. Preparation of 1-(3-(4-((7-(8-Methyl-2,3-dihydro-1H-pyrido[2,3-b][1,4]oxazin-7-yl)quinazolin-2-yl)amino)-1H-pyrazol-1-yl)azetidin-1-yl)propan-1-one.

A mixture containing K₃PO₄ (40 mg, 0.19 mmol), water (0.010 mL, 0.56 mmol), Pd₂(dba)₃ (12 mg, 0.013 mmol), t-BuBrettPhos (12 mg, 0.025 mmol), 1-(3-(4-bromo-1H-pyrazol-1-yl)azetidin-1-yl)propan-1-one (30 mg, 0.17 mmol) and tert-butyl 7-(2-aminoquinazolin-7-yl)-8-methyl-2,3-dihydro-1H-pyrido[2,3-b][1,4]oxazine-1-carboxylate (I-4) (25 mg, 0.064 mmol) in toluene (1 mL) was deoxygenated by bubbling argon for 3 min, warmed to 110° C. and stirred overnight. The mixture was filtered, and concentrated. The residue was dissolved in 1 mL of DCM, 0.2 mL of TFA was added, then the mixture was aged for 3 hours and concentrated. The residue was dissolved in 3 mL of MeOH and purified by reverse phase chromatography (gradient of 15-70% MeCN/water with 0.1% NH₄OH) to provide the desired product. ¹H NMR (500 MHz, DMSO-d₆) δ 9.83 (s, 1H), 9.25 (s, 1H), 8.42 (s, 1H), 7.91 (d, J=10 Hz, 1H), 7.76 (s, 1H), 7.56 (s, 1H), 7.39 (s, 1H), 7.26 (d, J=10 Hz, 1H), 5.71 (m, 1H), 5.28 (m, 1H), 4.55 (m, 1H), 4.41 (m, 1H), 4.29 (m, 3H), 4.13 (m, 1H), 3.38 (s, 2H), 2.12 (q, J=10 Hz, 2H), 2.04 (s, 3H), 1.00 (t, J=10 Hz, 3H); MS (EI) Calc'd for C₂₅H₂₇N₈O₂ [M+H]⁺, 471; found, 471.

### Example 1D. Preparation of 1-6

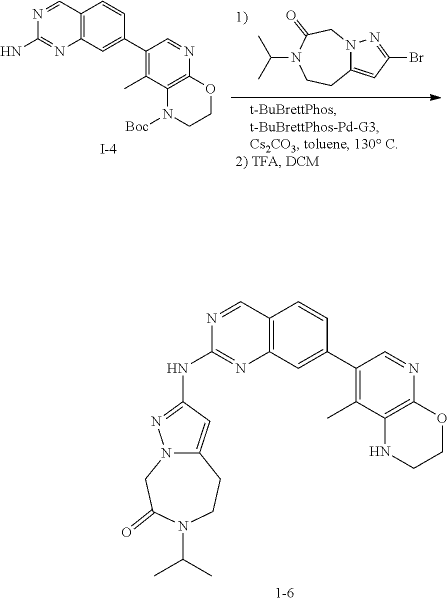

A mixture containing 2-bromo-6-isopropyl-5,6-dihydro-4H-pyrazolo[1,5-d][1,4]diazepin-7(8H)-one (30 mg, 0.11 mmol; see WO 2018/183964 for preparation), tert-butyl 7-(2-aminoquinazolin-7-yl)-8-methyl-2,3-dihydro-1H-pyrido[2,3-b][1,4]oxazine-1-carboxylate (I-4) (30 mg, 0.076 mmol), Cs₂CO₃ (50 mg, 0.15 mmol), t-BuBrettPhos (15 mg, 0.031 mmol), t-BuBrettPhos-Pd-G3 (25 mg, 0.029 mmol) in toluene (1 mL) was stirred for 16 hours at 130° C. The mixture was filtered and concentrated. The residue was dissolved in 1 mL of DCM and 1 mL of TFA and aged for 1 h. The mixture was concentrated, dissolved in 2 mL of DMSO, filtered, then purified by reverse phase chromatography (gradient of 15-70% MeCN/water with 0.1% NH₄OH) to provide 6-isopropyl-2-((7-(8-methyl-2,3-dihydro-1H-pyrido[2,3-b][1,4]oxazin-7-yl)quinazolin-2-yl)amino)-5,6-dihydro-4H-pyrazolo[1,5-d][1,4]diazepin-7(8H)-one. ¹H NMR (500 MHz, DMSO-d₆) δ 9.97 (s, 1H), 9.27 (s, 1H), 7.92 (d, J=10 Hz, 1H), 7.47 (s, 1H), 7.38 (s, 1H), 7.28 (d, J=10 Hz, 1H), 6.84 (s, 1H), 5.70 (s, 1H), 4.95 (s, 2H), 4.60 (m, 1H), 4.29 (br, 2H), 3.80 (br, 2H), 3.38 (br, 1H), 3.03 (br, 2H), 2.03 (s, 3H), 1.13 (2 d, J=5 Hz, 6H); MS (EI) Calc'd for C₂(H₂₉N₈O₂ [M+H]⁺, 485; found, 485.

### Example 1E. Preparation of 1-7

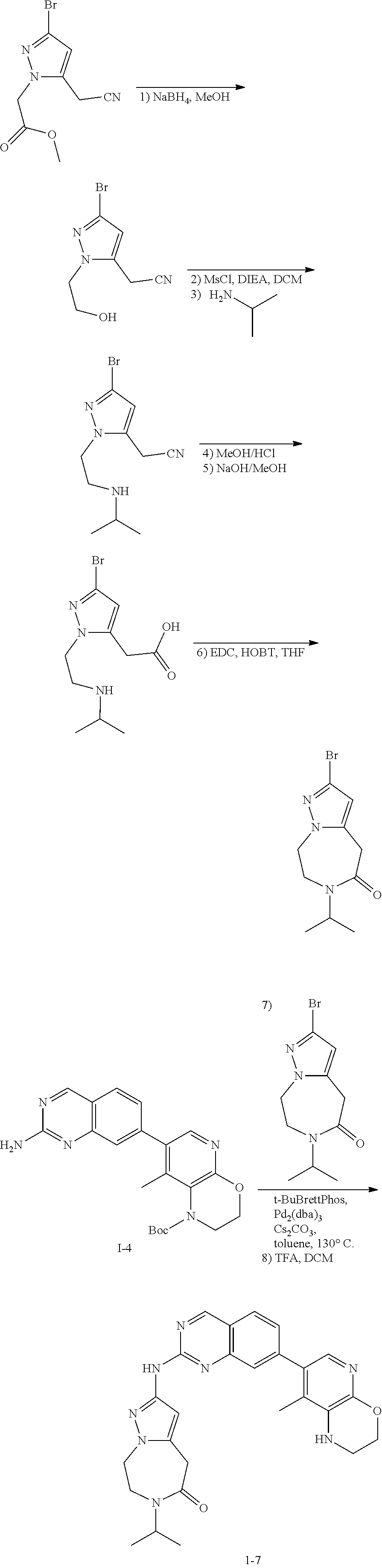

### Step 1. Preparation of 2-(3-bromo-1-(2-hydroxyethyl)-1H-pyrazol-5-yl)acetonitrile

To a mixture of methyl 2-(3-bromo-5-(cyanomethyl)-1H-pyrazol-1-yl)acetate (5 g, 19.37 mmol) in MeOH (40 mL) was added NaBH₄ (2.199 g, 58.1 mmol) portionwise at 0° C., then it was stirred at 0° C. for about 3 h. TLC showed most finished. It was quenched with H₂O (20 mL). The mixture was concentrated in vacuum to remove the MeOH. The residue was extracted with EtOAc (3×20 mL). The combined organic layers were washed with brine (20 mL), dried over anhydrous Na₂SO₄, filtered and the filtrate was evaporated in vacuum to give crude product. The residue was purified by flash silica gel chromatography (ISCO®; 20 g SepaFlash® Silica Flash Column, eluent of [0˜60]% ethyl acetate/pet, ether gradient at 35 mL/min) to give 2-(3-bromo-1-(2-hydroxyethyl)-1H-pyrazol-5-yl)acetonitrile as a solid. ¹H NMR (500 MH z, CDCl₃) δ 6.31 (s, 1H), 4.11 (t, J=5 Hz, 2H), 3.91 (t, J=5 Hz, 2H), 3.87 (s, 2H), 2.90 (s, 1H).

### Steps 2 and 3. Preparation of 2-(3-bromo-1-(2-(isopropylamino)ethyl)-1H-pyrazol-5-yl)acetonitrile

To a solution of 2-(3-bromo-1-(2-hydroxyethyl)-1H-pyrazol-5-yl)acetonitrile (2 g, 8.69 mmol), DIEA (3.04 mL, 17.39 mmol) in DCM (30 mL) was added MsCl (0.782 mL, 10.04 mmol). Then it was stirred at 0° C. for 30 min. TLC showed completed. Propan-2-amine (4.47 mL, 52.2 mmol) was added to the mixture. The mixture was sealed and stirred at 40° C. for 15 h. LC/MS showed completed. The mixture was dissolved in DCM (100 mL), washed with water (20 mL), dried over anhydrous Na₂SO₄, filtered and the filtrate was evaporated in vacuum to give the crude product 2-(3-bromo-1-(2-(isopropylamino)ethyl)-1H-pyrazol-5-yl)acetonitrile as an oil. ¹H NMR (500 MHz, CDCl₃) δ 6.29 (s, 1H), 4.08 (t, J=5.5 Hz, 2H), 3.96 (s, 2H), 3.01 (d, J=5.5 Hz, 2H), 2.70 (q, J=6.5 Hz, 1H), 0.98 (d, J=6.5 Hz, 6H).

### Steps 4 and 5. Preparation of 2-(3-bromo-1-(2-(isopropylamino)ethyl)-1H-pyrazol-5-yl)acetic

To a solution of 2-(3-bromo-1-(2-(isopropylamino)ethyl)-1H-pyrazol-5-yl)acetonitrile (2.3 g, crude) in MeOH (10 mL) was added hydrogen chloride (5 mL, 20.00 mmol) (4M in MeOH). Then it was stirred at 60° C. for 24 h. LC/MS showed most completed. It was concentrated to give crude methyl 2-(3-bromo-1-(2-(isopropylamino)ethyl)-1H-pyrazol-5-yl)acetate as a solid. The crude mixture was taken up in MeOH (60 mL) and added sodium hydroxide (12.82 mL, 25.6 mmol). Then it was stirred at 30° C. for 1 h. LC/MS showed the desired product. It was concentrated in vacuum to give crude of 2-(3-bromo-1-(2-(isopropylamino)ethyl)-1H-pyrazol-5-yl)acetic acid as a crude oil. MS (EI) Calc'd for C₁₀H₁₇BrN₃O₂[M+H]⁺, 290, 292; found, 290, 292.

### Step 6. Preparation of 2-bromo-6-isopropyl-7,8-dihydro-4H-pyrazolo[1,5-d][1,4]diazepin-5(6H)-one

To a mixture of 2-(3-bromo-1-(2-(isopropylamino)ethyl)-1H-pyrazol-5-yl)acetic acid (3.9 g, crude) in THF (1 L) was added DIEA (7.04 mL, 40.3 mmol), HOBT (4.12 g, 26.9 mmol) and EDC (5.15 g, 26.9 mmol). Then it was stirred at 20° C. for 1 h. LC/MS showed the desired product. The mixture was dissolved in water (100 mL) and EtOAc (400 mL). The organic layer was separated and the aqueous was re-extracted with EtOAc (3×400 mL). The combined organic layers were washed with brine (300 mL), dried over anhydrous Na₂SO₄, filtered and the filtrate was evaporated in vacuum to give the crude product. The residue was purified by flash silica gel chromatography (ISCO®; 40 g SepaFlash® Silica Flash Column, eluent of [0˜60]% ethyl acetate/pet, ether gradient at 40 mL/min) to give 2-bromo-6-isopropyl-7,8-dihydro-4H-pyrazolo[1,5-d][1,4]diazepin-5(6H)-one as a solid. ¹H NMR (500 MHz, CDCl₃) δ 6.08 (s, 1H), 4.86 (q, J=6.5 Hz, 1H), 4.26-4.33 (t. J=5.5 Hz, 2H), 3.77-3.83 (m, 4H), 1.18 (d, J=7.0 Hz, 6H).

### Step 7. Synthesis of Compound 2-7 2-{[7-(8-methyl-2,3-dihydro-1H-pyrido[2,3-b][1,4]oxazin-7-yl)quinazolin-2-yl]amino}-6-(propan-2-yl)-7,8-dihydro-4H-pyrazolo[1,5-d][1,4]diazepin-5(6H)-one

To a mixture of 2-bromo-6-isopropyl-7,8-dihydro-4H-pyrazolo[1,5-d][1,4]diazepin-5(6H)-one (27.7 mg, 0.102 mmol), Intermediate I-4 (40 mg, 0.102 mmol), Cs₂CO₃ (99 mg, 0.305 mmol) in toluene (1.5 mL) was added t-BuBrettPhos (14.78 mg, 0.031 mmol) and Pd₂(dba)₃ (12.10 mg, 0.013 mmol). Then it was stirred at 130° C. for 6 h under nitrogen. LC/MS showed desired product. The mixture was purified by flash silica gel chromatography (Pet, ether:EtOAc=1:1 to EtOAc:MeOH=8:1) to give the protected adduct as an oil. The compound was then taken up in DCM (3 mL) and added TFA (1.5 mL). Then it was stirred at 10° C. for 1 h. The mixture was concentrated to give crude material which was purified by HPLC (Column YMC-Actus Triart C18 150×30 mm×5 um; 18-48% MeCN/water (with 0.1% TFA) to give compound I-7 as a solid. ¹H NMR (400 MHz, DMSO-d₆) δ 10.20 (s, 1H), 9.30 (s, 1H), 7.95 (d, J=8.4 Hz, 1H), 7.56 (s, 1H), 7.47 (s, 1H), 7.30 (dd, J=8.4, 1.6 Hz, 1H), 6.76 (s, 1H), 4.61 (q, J=6.4 Hz, 1H), 4.36 (t, J=4.4 Hz, 2H), 4.10 (t, J=4.4 Hz, 2H), 3.91 (t, =4.4 Hz, 2H), 3.87 (s, 2H), 3.41 (br d, J=3.6 Hz, 2H), 2.05 (s, 3H), 1.10 (d, J=6.4 Hz, 6H); MS (EI) Calc'd for C₂₆H₂₉N₈O₂ [M+H]⁺, 485; found, 485.

Compounds listed in the table below were prepared using the synthetic methods described in the aforementioned examples. The table provides the compound structure, the name, the calculated and observed masses, and the example method used for preparation.

TABLE 1

| | | | Exact |
|---|---|---|---|
| | | | Mass |
| Example | | | [M + H]⁺; |
| Number | Structure | Name | Method |
| 1-1 | 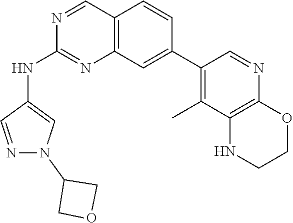 [SMILES: Cc1c(-c2ccc3cnc(Nc4cnn(C5COC5)c4)nc3c2)cnc2c1NCCO2] | 7-(8-methyl-2,3-dihydro- 1H-pyrido[2,3- b][1,4]oxazin-7-yl)-N-[1- (oxetan-3-yl)-1H-pyrazol- 4-yl]quinazolin-2-amine | Calc'd 416, found 416; 1A |
| 1-2 | 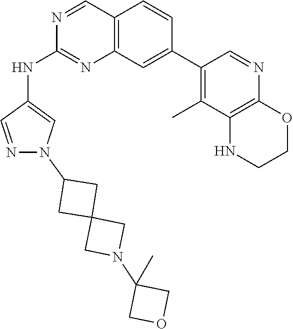 [SMILES: Cc1c(-c2ccc3cnc(Nc4cnn(C5CC6(C5)CN(C5(C)COC5)C6)c4)nc3c2)cnc2c1NCCO2] | 7-(8-methyl-2,3-dihydro- 1H-pyrido[2,3- b][1,4]oxazin-7-yl)-N-{1- [2-(3-methyloxetan-3-yl)- 2-azaspiro[3.3]heptan-6- yl]-1H-pyrazol-4- yl}quinazolin-2-amine | Calc'd 525, found 525; 1B |
| 1-3 | 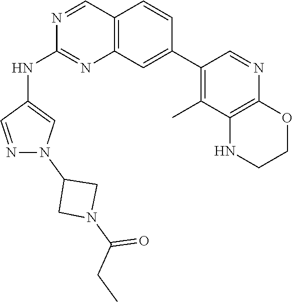 [SMILES: CCC(=O)N1CC(n2cc(Nc3ncc4ccc(-c5cnc6c(c5C)NCCO6)cc4n3)cn2)C1] | 1-[3-(4-{[7-(8-methyl-2,3- dihydro-1H-pyrido[2,3- b][1,4]oxazin-7- yl)quinazolin-2-yl]amino}- 1H-pyrazol-1-yl)azetidin- 1-yl]propan-1-one | Calc'd 471, found 471; 1C |
| 1-4 | 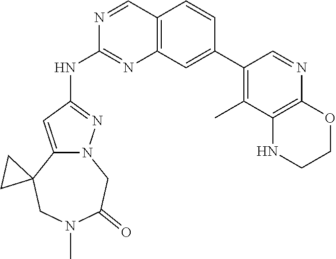 [SMILES: Cc1c(-c2ccc3cnc(Nc4cc5n(n4)CC(=O)N(C)CC54CC4)nc3c2)cnc2c1NCCO2] | 6′-methyl-2′-{[7-(8-methyl- 2,3-dihydro-1H- pyrido[2,3-b][1,4]oxazin- 7-yl)quinazolin-2- yl]amino}-5′,6′- dihydrospiro[cyclopropane- 1,4′-pyrazolo[1,5- d][1,4]diazepin]-7′(8′H)- one | Calc'd 483, found 483; 1D |
| 1-5 | 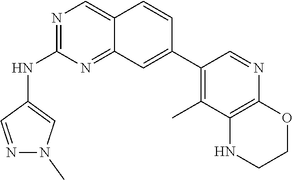 [SMILES: Cc1c(-c2ccc3cnc(Nc4cnn(C)c4)nc3c2)cnc2c1NCCO2] | 7-(8-methyl-2,3-dihydro- 1H-pyrido[2,3- b][1,4]oxazin-7-yl)-N-(1- methyl-1H-pyrazol-4- yl)quinazolin-2-amine | Calc'd 374, found 374; 1A |
| 1-6 | 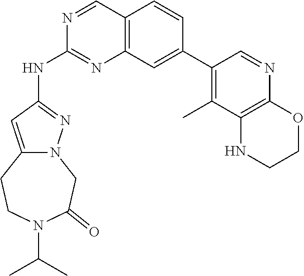 [SMILES: Cc1c(-c2ccc3cnc(Nc4cc5n(n4)CC(=O)N(C(C)C)CC5)nc3c2)cnc2c1NCCO2] | 2-{[7-(8-methyl-2,3- dihydro-1H-pyrido[2,3- b][1,4]oxazin-7- yl)quinazolin-2-yl]amino}- 6-(propan-2-yl)-5,6- dihydro-4H-pyrazolo[1,5- d][1,4]diazepin-7(8H)-one | Calc'd 485, found 485; 1D |
| 1-7 | 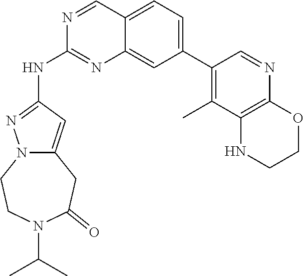 [SMILES: Cc1c(-c2ccc3cnc(Nc4cc5n(n4)CCN(C(C)C)C(=O)C5)nc3c2)cnc2c1NCCO2] | 2-{[7-(8-methyl-2,3- dihydro-1H-pyrido[2,3- b][1,4]oxazin-7- yl)quinazolin-2-yl]amino}- 6-(propan-2-yl)-7,8- dihydro-4H-pyrazolo[1,5- d][1,4]diazepin-5(6H)-one | Calc'd 485, found 485; 1E |
| 1-8 | 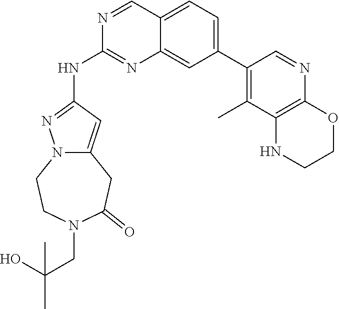 [SMILES: Cc1c(-c2ccc3cnc(Nc4cc5n(n4)CCN(CC(C)(C)O)C(=O)C5)nc3c2)cnc2c1NCCO2] | 6-(2-hydroxy-2- methylpropyl)-2-((7-(8- methyl-2,3-dihydro-1H- pyrido[2,3-b][1,4]oxazin- 7-yl)quinazolin-2- yl)amino)-7,8-dihydro-4H- pyrazolo[1,5- d][1,4]diazepin-5(6H)-one | Calc'd 515, found 515; 1E |

### COMPOUND EXAMPLES OF TABLE 2

### Example 2A. Preparation of 2-3

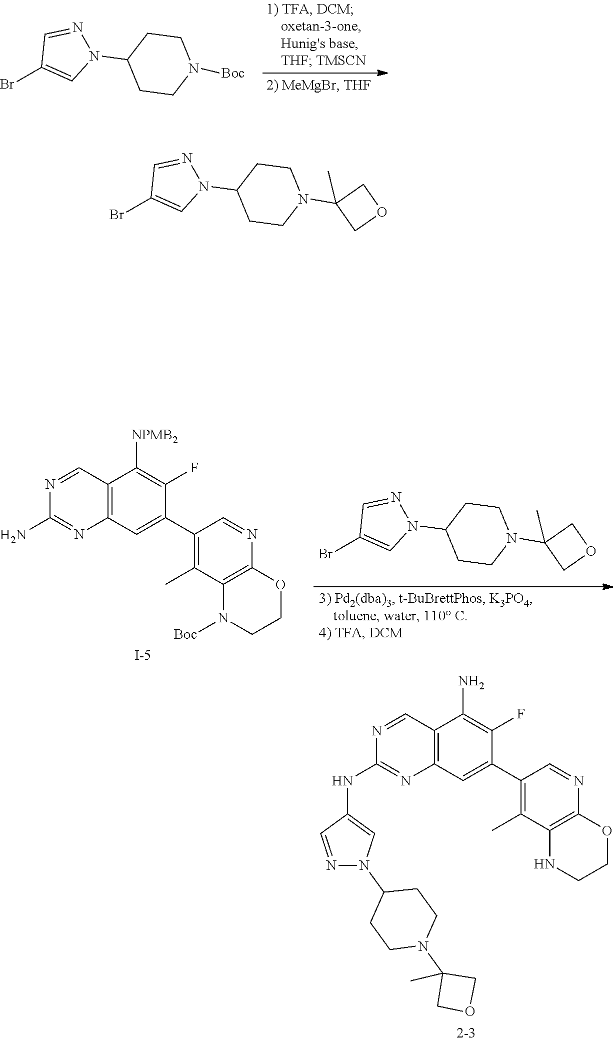

### Step 1. Preparation of 3-(4-(4-Bromo-1H-pyrazol-1-yl)piperidin-1-yl)oxetane-3-carbonitrile

A solution of tert-butyl 4-(4-bromo-1H-pyrazol-1-yl)piperidine-1-carboxylate (1.5 g, 4.54 mmol) in a 20 mL of DCM and TFA (0.3 mL) was stirred for 4 h, then concentrated to a solid. The solid residue was dissolved in DCE (11 mL), treated with Hunig's base (4.0 mL, 23 mmol) and oxetan-3-one (0.67 mL, 11 mmol). The reaction mixture was stirred at room temperature for 20 min, after which trimethylsilanecarbonitrile (0.68 mL, 5.5 mmol) and acetic acid (0.31 mL, 5.5 mmol) were added. The reaction mixture was heated to 70° C. and stirred overnight. The crude reaction mixture was concentrated and partitioned between DCM and sat'd NaHCO₃. The combined organic layer was dried (Na₂SO₄) and concentrated to afford 3-(4-(4-bromo-1H-pyrazol-1-yl)piperidin-1-yl)oxetane-3-carbonitrile. MS (EI) calc'd for Cl₂H₁₆BrN₄O [M+H]⁺, 311, 313; found, 311, 313.

### Step 2. Preparation of 4-(4-Bromo-1H-pyrazol-1-yl)-1-(3-methyloxetan-3-yl)piperidine

A 20-mL vial with 3-(4-(4-bromo-1H-pyrazol-1-yl)piperidin-1-yl)oxetane-3-carbonitrile (700 mg, 2.25 mmol) in THF (5.2 mL) was treated with 3.4 M ether solution of MeMgBr (3.3 mL, 11 mmol). The reaction was heated to 65° C. for 3 h, then quenched with 1 M NaOH. The aqueous phase was extracted with DCM, and the combined organic layers dried over Na₂SO₄, filtered, and concentrated. The crude residue was purified by column chromatography (0-100% EtOAc/hexanes) to give 4-(4-bromo-1H-pyrazol-1-yl)-1-(3-methyloxetan-3-yl)piperidine as an oil. MS (EI) calc'd for C₁₂H₁₉BrN₃O [M+H]⁺, 300, 302; found, 300, 302.

### Steps 3 and 4. Preparation of 6-Fluoro-7-(8-methyl-2,3-dihydro-1H-pyrido[2,3-b][1,4]oxazin-7-yl)-N²-(1-(1-(3-methyloxetan-3-yl)piperidin-4-yl)-1H-pyrazol-4-yl)quinazoline-2,5-diamine (2-3)

A mixture containing K₃PO₄ (40 mg, 0.19 mmol), Pd₂(dba)₃ (12 mg, 0.013 mmol), t-BuBrettPhos (10 mg, 0.021 mmol), water (20 μL, 1.1 mmol), tert-butyl 7-(2-amino-5-(bis(4-methoxybenzyl)amino)-6-fluoroquinazolin-7-yl)-8-methyl-2,3-dihydro-11H-pyrido[2,3-b][1,4]oxazine-1-carboxylate (I-5) (30 mg, 0.045 mmol) in toluene (1 mL) was treated with 4-(4-bromo-1H-pyrazol-1-yl)-1-(3-methyloxetan-3-yl)piperidine (35 mg, 0.12 mmol). Deoxygenated by bubbling argon for 3 min, then warmed to 110° C. and stirred overnight. Cooled to RT, filtered and concentrated. Crude reaction residue was dissolved in 1 mL of DCM, 1 mL of TFA, aged for 2 h and concentrated. Crude reaction was then dissolved in 2 mL of DMSO, filtered and purified by reverse phase chromatography (gradient of 2-55% MeCN/water with 0.1% TFA) to provide 6-fluoro-7-(8-methyl-2,3-dihydro-1H-pyrido[2,3-b][1,4]oxazin-7-yl)-N²-(1-(1-(3-methyloxetan-3-yl)piperidin-4-yl)-1H-pyrazol-4-yl)quinazoline-2,5-diamine. ¹H NMR (500 MHz, DMSO-d₆) δ 10.5 (br, 1H), 9.70 (s, 1H), 9.52 (s, 1H), 8.25 (s, 1H), 7.63 (s, 1H), 7.39 (s, 1H), 6.61 (s, 1H), 4.84 (m, 2H), 4.52 (m, 1H), 4.39 (m, 2H), 4.33 (m, 2H), 3.32-3.42 (m, 4H), 3.17 (m, 2H), 2.24-2.34 (m, 4H), 1.95 (s, 3H), 1.65 (s, 3H); MS (EI) calc'd for C₂H₃₃FN₉O₂, [M+H]⁺, 546; found, 546.

### Example 2B. Synthesis of Compound 2-8

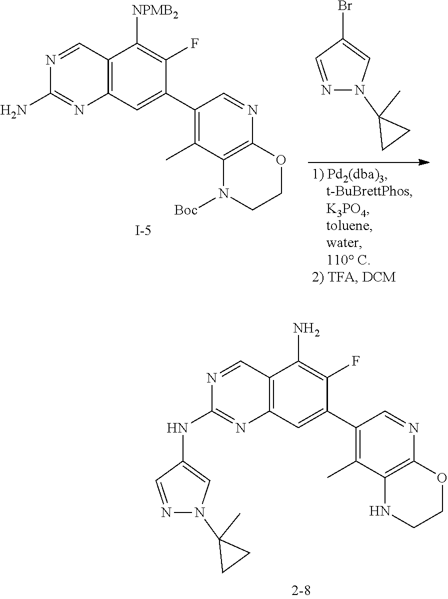

A mixture containing K₃PO₄ (25 mg, 0.118 mmol), Pd₂(dba)₃ (5 mg, 0.005 mmol), t-BuBrettPhos (8 mg, 0.02 mmol), water (20 μL, 1.1 mmol), tert-butyl 7-(2-amino-5-(bis(4-methoxybenzyl)amino)-6-fluoroquinazolin-7-yl)-8-methyl-2,3-dihydro-1H-pyrido[2,3-b][1,4]oxazine-1-carboxylate (I-5) (25 mg, 0.037 mmol) in toluene (1 mL) was treated with 4-bromo-1-(1-methylcyclopropyl)-1H-pyrazole (20 mg, 0.099 mmol). The reaction mixture was deoxygenated by bubbling argon for 3 min, then warmed to 110° C. and stirred overnight. The reaction mixture was cooled to RT, filtered and concentrated. The crude reaction residue was dissolved in 1 mL of DCM, 1 mL of TFA, aged for 2 h and concentrated. The residue was dissolved in 2 mL of DMSO, filtered and purified by reverse phase chromatography (gradient of 2-55% MeCN/water with 0.1% NH₄OH) to provide 6-fluoro-7-(8-methyl-2,3-dihydro-1H-pyrido[2,3-b][1,4]oxazin-7-yl)-N²-(1-(1-methylcyclo-propyl)-1H-pyrazol-4-yl)quinazoline-2,5-diamine. ¹H NMR (500 MHz, DMSO-d₆) δ 9.53 (s, 1H), 9.48 (s, 1H), 8.17 (br, 1H), 7.54 (s, 1H), 7.35 (s, 1H), 6.64 (s, 1H), 6.27 (s, 2H), 5.68 (s, 1H), 4.29 (br, 2H), 3.37 (br, 2H), 1.94 (s, 3H), 1.55 (s, 3H), 1.16 (m, 2H), 0.88 (n, 2H); MS (EI) calc'd for C₂₃H₂₄FN₈O [M+H]⁺, 447; found, 447.

### Example 2C. Preparation of 2-10

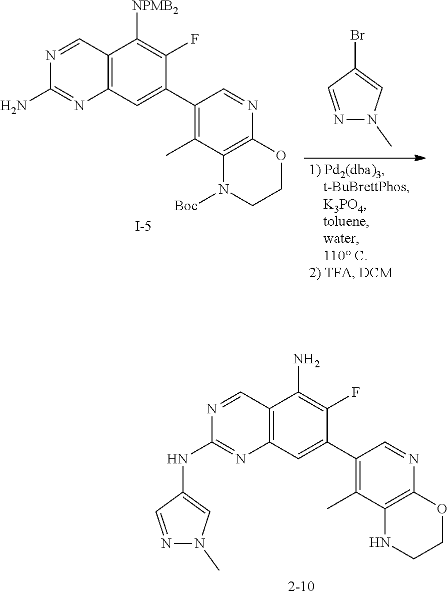

A mixture containing K₃PO₄ (25 mg, 0.12 mmol), Pd₂(dba)₃ (5.0 mg, 5.5 μmol), t-BuBrettPhos (8.0 mg, 0.017 mmol), Intermediate I-5 (25 mg, 0.037 mmol) and 4-bromo-1-methyl-1H-pyrazole (12 mg, 0.075 mmol) in toluene (1 mL) was treated with water (0.020 mL, 1.1 mmol) and deoxygenated by bubbling argon gas through the reaction mixture for 3 min. The mixture was stirred overnight at 110° C. After cooling, the reaction was filtered and concentrated to dryness. The crude mixture was dissolved in 1 mL of DCM and treated with 1 mL of TFA. The solution allowed to age for 2 h, then concentrated to dryness. The residue was purified by reverse phase chromatography (gradient of 2-55% MeCN/water with 0.1% NH₄OH) to provide 2-10 as a solid. ¹H NMR (500 MHz, DMSO-d₆) δ 9.51 (2 s, 2H), 8.17 (br s, 1H), 7.53 (s, 1H), 7.35 (s, 1H), 6.65 (s, 1H), 6.28 (s, 2H), 5.69 (s, 1H), 4.30 (t, J=4.2 Hz, 2H), 3.81 (br, 4H), 3.38 (s, 2H), 1.94 (s, 3H); MS (EI) calc'd for C₂₀H₂₀FN₈O [M+H]⁺, 407; found, 407.

### Example 2D. Preparation of 2-11

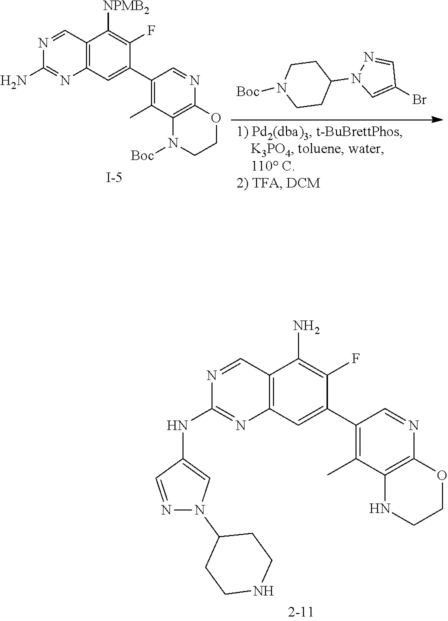

A mixture containing K₃PO₄ (40 mg, 0.19 mmol), Pd₂(dba)₃ (12 mg, 0.013 mmol), t-BuBrettPhos (10 mg, 0.021 mmol), Intermediate I-5 (30 mg, 0.045 mmol) and tert-butyl 4-(4-bromo-1H-pyrazol-1-yl)piperidine-1-carboxylate (30 mg, 0.091 mmol) in toluene (1 mL) was treated with water (0.020 mL, 1.1 mmol) and deoxygenated by bubbling argon gas through the reaction mixture for 3 min. The mixture was stirred overnight at 110° C. After cooling, the reaction is filtered and concentrated to dryness. The crude mixture was dissolved in 1 mL of DCM and treated with 1 mL of TFA. The solution allowed to age for 2 h, then concentrated to dryness. The residue was purified by reverse phase chromatography (gradient of 2-55% MeCN/water with 0.1% TFA) to provide 6-fluoro-7-(8-methyl-2,3-dihydro-1H-pyrido[2,3-b][1,4]oxazin-7-yl)-N²-(1-(piperidin-4-yl)-1H-pyrazol-4-yl)quinazoline-2,5-diamine (4-11). ¹H NMR (500 MHz, DMSO-d₆) δ 9.54 (s, 1H), 9.48 (s, 1H), 8.21 (s, 1H), 7.55 (s, 1H), 7.35 (s, 1H), 6.65 (s, 1H), 6.27 (s, 2H), 5.69 (s, 1H), 4.29 (m, 2H), 4.16 (m, 1H), 3.37 (m, 2H), 3.04 (m, 2H), 2.57 (m, 2H), 1.94 (s, 3H), 1.91 (s, 2H), 1.76 (m, 2H); MS (EI) calc'd for C₂₄H₂₇FN₉O [M+H]⁺, 476; found, 476.

### Example 2E. Preparation of 2-15

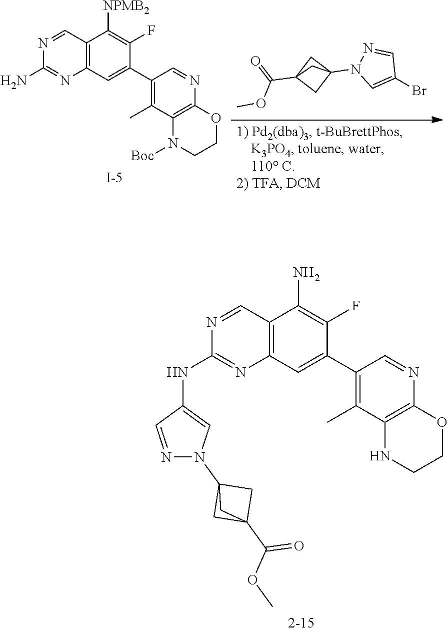

To a vial containing Intermediate I-5 (40 mg, 0.060 mmol) was added methyl 3-(4-bromo-1H-pyrazol-1-yl)bicyclo[1.1.1]pentane-1-carboxylate (32.5 mg, 0.120 mmol), Pd₂(dba)₃ (10.99 mg, 0.012 mmol), t-BuBrettPhos (5.82 mg, 0.012 mmol) and K₃PO₄ (38.2 mg, 0.180 mmol). The mixture was dissolved in toluene (1 mL) and water (10 μL) and deoxygenated by bubbling argon gas through the reaction mixture for 3 min. The mixture was stirred overnight at 110° C. After cooling, the reaction is filtered and concentrated to dryness. The crude mixture was dissolved in 1 mL of DCM and treated with 1 mL of TFA. The reaction was heated to 65° C. for 1 h, then concentrated to dryness. The residue was purified by reverse phase chromatography (gradient elution of 2-55% Acetonitrile/Water+0.1% TFA) to provide compound 2-15. ¹H NMR (500 MHz, DMSO-d) δ 9.85 (s, 1H), 9.56 (s, 1H), 8.21 (s, 1H), 7.64 (s, 1H), 7.51 (s, 1H), 6.72 (s, 1H), 4.43 (s, 3H), 3.67 (s, 3H), 3.45 (s, 2H), 2.01 (s, 3H); MS (EI) calc'd for C₂₆H₂₆FN₈O₃ [M+H]⁺, 517; found, 517.

### Example 2F. Preparation of Compounds 2-19 and 2-21

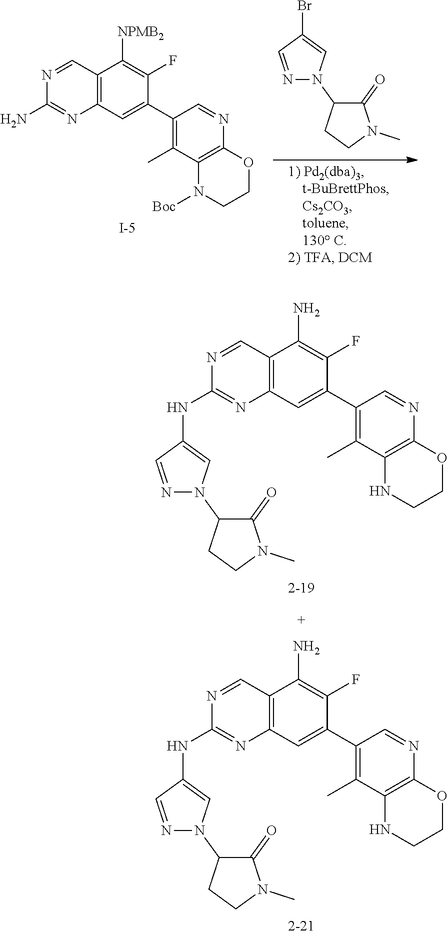

To a solution of Intermediate I-5 (150 mg, 0.225 mmol), 3-(4-bromo-1H-pyrazol-1-yl)-1-methylpyrrolidin-2-one (71 mg, 0.29 mmol; see WO2018/183956 for preparation), Cs₂CO₃ (220 mg, 0.675 mmol). Pd₂(dba)₃ (26.8 mg, 0.029 mmol) and t-BuBrettPhos (33 mg, 0.067 mmol) in toluene (4 mL). Then it was stirred at 125° C. for 6 h under nitrogen. LCMS showed the desired product was formed. The mixture was quenched with water (20 mL), then the mixture was extracted with EtOAc (3×30 mL), the combined organic layers were washed with brine, dried over Na₂SO₄, filtered and concentrated to get the residue. It was further purified by flash silica gel chromatography (gradient of 0-25% EtOAc/MeOH, 4 g silica) to give tert-butyl 7-(5-(bis(4-methoxybenzyl)amino)-6-fluoro-2-((1-(I-methyl-2-oxopyrrolidin-3-yl)-1H-pyrazol-4-yl)amino)quinazolin-7-yl)-8-methyl-2,3-dihydro-1H-pyrido[2,3-b][1,4]oxazine-1-carboxylate as an oil. The product was taken up in DCM (2 mL) and TFA (2 mL) and then stirred at 25° C. for 1 h. LCMS showed the desired product was formed. The mixture was concentrated to give the residue which was separated by HPLC(TFA) to give the desired racemate compound as a solid. Prep-HPLC condition: Column YMC-Actus Triart C18 150×30 mm×5 um; 22-52% MeCN/water with 0.1% TFA). The enantiomers were then separated by SFC (Instrument SFC-16, Column DAICEL CHIRALPAK AS (250 mm×30 mm, 10 um), 50% EtOH/CO₂ with 0.1% NH₃H₂O) to give Compound 2-19 (Rt=1.888 min) and Compound 2-21 (Rt=3.274 min) as solids.

### Compound 2-19. (S or R)-3-(4-{[5-Amino-6-fluoro-7-(8-methyl-2,3-dihydro-1H-pyrido[2,3-b][1,4]oxazin-7-yl)quinazolin-2-yl]amino}-1H-pyrazol-1-yl)-1-methylpyrrolidin-2-one

¹H NMR (400 MHz, MeOD) δ 9.37 (s, 1H), 8.34 (s, 1H), 7.72 (s, 1H), 7.37 (s, 1H), 6.79 (d. J=5.6 Hz, 1H), 5.14 (t, J=9.2 Hz, 1H), 4.36-4.42 (m, 2H), 3.46-3.65 (m, 4H), 2.94 (s, 3H), 2.60-2.71 (m, 1H), 2.44-2.54 (m, 1H), 2.03 (d, J=1.2 Hz, 3H); MS (EI) calc'd for C₂₄H₂₅FN₉O₂ [M+H]⁺, 490; found, 490.

### Compound 2-21. (S or R)-3-(4-{[5-Amino-6-fluoro-7-(8-methyl-2,3-dihydro-1H-pyrido[2,3-b][1,4]oxazin-7-yl)quinazolin-2-yl]amino}-1H-pyrazol-1-yl)-1-methylpyrrolidin-2-one

¹H NMR (500 MHz, DMSO-d₆) δ 9.47 (s, 1H), 8.26 (s, 1H), 7.75 (s, 1H), 7.40 (s, 1H), 6.77 (d, J=5.6 Hz, 1H), 5.17 (t, J=9.6 Hz, 1H), 4.44-4.48 (m, 2H), 3.52-3.66 (m, 4H), 2.94 (s, 3H), 2.65-2.67 (m, 1H), 2.44-5.53 (m, 1H), 2.06 (d, J=1.2 Hz, 3H); MS (EI) calc'd for C₂₄H₂₅FN₉O₂ [M+H]⁺, 490; found, 490.

### Example 2G. Synthesis of Compound 2-43

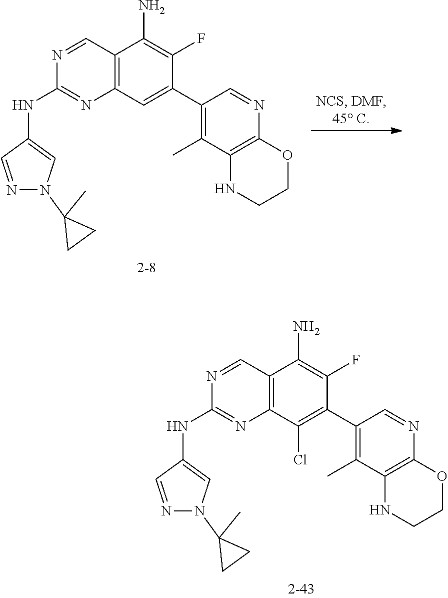 [SMILES: Cc1c(-c2cc3nc(Nc4cnn(C5(C)CC5)c4)ncc3c(N)c2F)cnc2c1NCCO2 ; Cc1c(-c2c(F)c(N)c3cnc(Nc4cnn(C5(C)CC5)c4)nc3c2Cl)cnc2c1NCCO2]

A solution of 6-fluoro-7-(8-methyl-2,3-dihydro-1H-pyrido[2,3-b][1,4]oxazin-7-yl)-N²-(1-(1-methylcyclopropyl)-1H-pyrazol-4-yl)quinazoline-2,5-diamine (2-8; 60 mg, 0.11 mmol) in DMF (1 mL) was treated with NCS (17 mg, 0.13 mmol) and the mixture stirred for 1 hour at 45° C.

It was then diluted with DMSO (1 mL), filtered, and purified by reverse phase chromatography (gradient of 15-70% MeCN/water with 0.1% TFA) to provide the desired product as a TFA salt. The product, (R and S)-8-chloro-6-fluoro-7-(8-methyl-2,3-dihydro-1H-pyrido[2,3-b][1,4]oxazin-7-yl)-N2-(1-(1-methylcyclopropyl)-1H-pyrazol-4-yl)quinazoline-2,5-diamine, was obtained as a mixture of atropstereoisomers. ¹H NMR (500 MHz, DMSO-d₆) δ 9.96 (s, 1H), 9.56 (s, 1H), 8.49 (s, 1H), 7.65 (s, 1H), 7.39 (s, 1H), 4.38 (m, 2H), 3.41 (m, 2H), 1.89 (s, 3H), 1.55 (s, 3H), 1.14 (m, 2H), 0.89 (m, 2H); MS (EI) calc'd for C₂₃H₂₃ClFN₈O [M+H]⁺, 481; found, 481.

### Example 2H. Synthesis of Compound 2-44

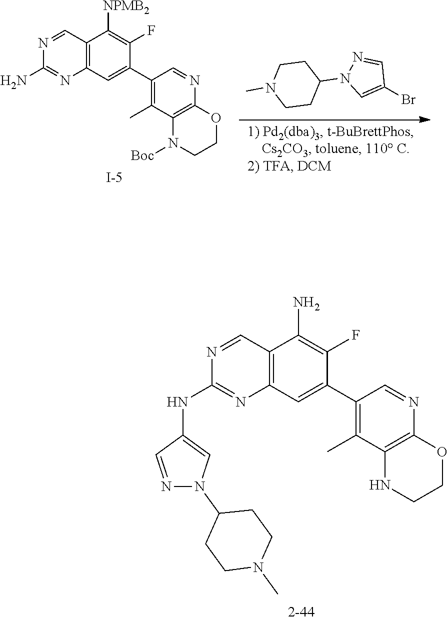

A mixture containing Cs₂CO₃ (400 mg, 1.23 mmol), Pd₂(dba)₃ (40 mg, 0.044 mmol), t-BuBrettPhos (44 mg, 0.091 mmol), 4-(4-bromo-1H-pyrazol-1-yl)-1-methylpiperidine (200 mg, 0.82 mmol), tert-butyl 7-(2-amino-5-(bis(4-methoxybenzyl)amino)-6-fluoroquinazolin-7-yl)-8-methyl-2,3-dihydro-1H-pyrido[2,3-b][1,4]oxazine-1-carboxylate (I-5; 300 mg, 0.450 mmol) in toluene (4 mL) was deoxygenated by bubbling nitrogen for 3 min. The mixture was warmed to 110° C., stirred for 20 hours, filtered, purified by chromatography on SiO₂ (0-50% MeOH/DCM; 80 g silica) to provide the desired intermediate as an oil, dissolved in 2 mL of DCM and 2 mL of TFA, aged 2 hours, and concentrated. The residue was dissolved in 4:1 DCM/MeOH and extracted with 2 N NaOH. The organic layer was then loaded onto a silica gel column (40 g silica) and eluted with 50% MeOH/DCM to provide the desired product as a solid. ¹H NMR (500 MHz, DMSO-d₆) δ 9.52 (s, 1H), 9.48 (s, 1H), 8.23 (s, 1H), 7.56 (s, 1H), 7.35 (s, 1H), 6.66 (s, 1H), 6.26 (br, 2H), 5.68 (s, 1H), 4.29 (m, 2H), 4.07 (m, 1H), 2.84 (m, 2H), 2.19 (s, 3H), 1.90-2.05 (m, 6H), 1.94 (s, 3H); MS (EI) calc'd for C₂₅H₂₉FN₉O [M+H], 490; found, 490.

### Example 21. Synthesis of Compound 2-28

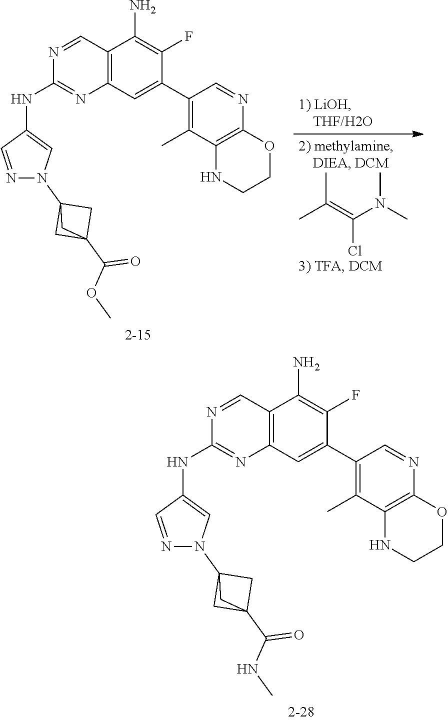

### Step 1. Preparation of 3-(4-((5-(bis(4-methoxybenzyl)amino)-7-(1-(tert-butoxycarbonyl)-8-methyl-2,3-dihydro-1H-pyrido[2,3-b][1,4]oxazin-7-yl)-6-fluoroquinazolin-2-yl)amino)-3-methyl-1H-pyrazol-1-yl)bicyclo[1.1.1]pentane-1-carboxylic acid

To a vial containing tert-butyl 7-(5-(bis(4-methoxybenzyl)amino)-6-fluoro-2-((1-(3-(methoxycarbonyl)bicyclo[1.1.1]pentan-1-yl)-1H-pyrazol-4-yl)amino)quinazolin-7-yl)-8-methyl-2,3-dihydro-1H-pyrido[2,3-b][1,4]oxazine-1-carboxylate (56.1 mg, 0.0655 mmol) was added lithium hydroxide (7.8 mg, 0.33 mmol). The mixture was dissolved in a mixture of THF (260 μL) and Water (66 μL) and heated to 50° C. for 1 h. The mixture was diluted with saturated aqueous NH₄Cl and DCM. The layers were separated and the aqueous layer was extracted with DCM and then 3:1 CHCl₃:IPA 3×. The combined organics were dried over Na₂SO₄, filtered and concentrated under reduced pressure to afford 3-(4-((5-(bis(4-methoxybenzyl)amino)-7-(I-(tert-butoxycarbonyl)-8-methyl-2,3-dihydro-1H-pyrido[2,3-b][1,4]oxazin-7-yl)-6-fluoroquinazolin-2-yl)amino)-1H-pyrazol-1-yl)bicyclo[1.1.1]pentane-1-carboxylic acid as a solid. MS (EI) calc'd for C₄₆H₄₇FN₈O₇ [M+H]⁺ 843; found 843.

### Step 2. Preparation of 3-(4-((5-amino-6-fluoro-7-(8-methyl-2,3-dihydro-1H-pyrido[2,3-b][1,4]oxazin-7-yl)quinazolin-2-yl)amino)-1H-pyrazol-1-yl)-N-methylbicyclo[1.1.1]pentane-1-carboxamide (2-28)

To a vial containing 3-(4-((5-(bis(4-methoxybenzyl)amino)-7-(1-(tert-butoxycarbonyl)-8-methyl-2,3-dihydro-1H-pyrido[2,3-b][1,4]oxazin-7-yl)-6-fluoroquinazolin-2-yl)amino)-1H-pyrazol-1-yl)bicyclo[1.1.1]pentane-1-carboxylic acid (83.7 mg, 0.0993 mmol) in DCM (662 μl) was added 1-chloro-N,N,2-trimethylpropenylamine (26 μL, 0.20 mmol). The mixture was allowed to stir for 10 min at 25° C. To the reaction mixture was added methylamine (99 μL, 2.0 M in THF, 0.20 mmol) and DIEA (35 μL, 0.20 mmol), sequentially. The reaction was then allowed to stir for 10 min. The reaction was concentrated under reduced pressure. The residue was dissolved in 1 mL of DCM and was added 1 mL of TFA. The reaction was allowed to stir at 65° C. for 10 min. The reaction mixture was concentrated under reduced pressure. The residue was dissolved in DMSO and filtered. The solution was purified by reverse phase column chromatography (gradient elution of 2-55% Acetonitrile/Water+0.1% TFA) to afford 2-28 as a solid. ¹H NMR (500 MHz, DMSO-d₆) δ 9.86 (s, 1H), 9.56 (s, 1H), 8.17 (s, 1H), 7.90 (d, J=4.6 Hz, 1H), 7.64 (s, 1H), 7.53 (s, 1H), 6.69 (s, 1H), 4.44 (m, 2H), 3.46 (m, 2H), 2.60 (d, J=4.6 Hz, 3H), 2.39 (s, 6H), 2.01 (s, 3H). MS (EI) calc'd for C₂₆H₂₇FN₉O₂ [M+H]⁺ 516; found 516.

### Example 2J. Synthesis of Compound 2-45

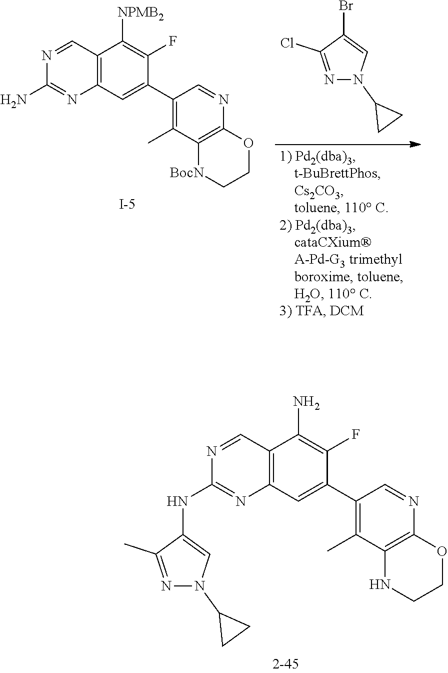

### Step 1. Preparation of tert-butyl 7-(5-(bis(4-methoxybenzyl)amino)-2-((5-chloro-1-cyclopropyl-1H-pyrazol-4-yl)amino)-6-fluoroquinazolin-7-yl)-8-methyl-2,3-dihydro-1H-pyrido[2,3-b][1,4]oxazine-1-carboxylate

To a vial containing tert-butyl 7-(2-amino-5-(bis(4-methoxybenzyl)amino)-6-fluoroquinazolin-7-yl)-8-methyl-2,3-dihydro-1H-pyrido[2,3-b][1,4]oxazine-1-carboxylate (100 mg, 0.150 mmol) was added 4-bromo-5-chloro-1-cyclopropyl-1H-pyrazole (49.8 mg, 0.225 mmol), Pd₂(dba)₃ (27.5 mg, 0.030 mmol), t-BuBrettPhos (29.1 mg, 0.060 mmol) and Cs₂CO₃ (147 mg, 0.450 mmol). The mixture was dissolved in toluene (1.5 mL) and degassed with N₂ for 5 min. The reaction was heated to 100° C. and allowed to stir overnight. The reaction mixture was filtered and concentrated under reduced pressure. The crude residue was purified by normal phase column chromatography (gradient elution of 0-100% EtOAc/CH₂Cl₂) to afford tert-butyl 7-(5-(bis(4-methoxybenzyl)amino)-2-((5-chloro-1-cyclopropyl-1H-pyrazol-4-yl)amino)-6-fluoroquinazolin-7-yl)-8-methyl-2,3-dihydro-1H-pyrido[2,3-b][1,4]oxazine-1-carboxylate as an oil. MS (EI) calc'd for C₄₃H₄₄ClFN₈O₅ [M+H]⁺, 807, 809; found, 807, 809.

### Step 2. Preparation of 2-45 (N²-(1-cyclopropyl-3-methyl-1H-pyrazol-4-yl)-6-fluoro-7-(8-methyl-2,3-dihydro-1H-pyrido[2,3-b][1,4]oxazin-7-yl)quinazoline-2,5-diamine)

To a vial containing tert-butyl 7-(5-(bis(4-methoxybenzyl)amino)-2-((5-chloro-1-cyclopropyl-1H-pyrazol-4-yl)amino)-6-fluoroquinazolin-7-yl)-8-methyl-2,3-dihydro-1H-pyrido[2,3-b][1,4]oxazine-1-carboxylate (115.4 mg, 0.143 mmol) was added trimethylboroxine (35.9 mg, 0.286 mmol), t-BuBrettPhos-Pd-G3 (10.41 mg, 0.014 mmol) and tripotassium phosphate (91 mg, 0.43 mmol). The mixture was dissolved in toluene (1.4 mL) and water (68 μL). The solution was degassed with N₂ for 5 min. The reaction mixture was heated to 100° C. and allowed to stir for 14 h. The reaction mixture was filtered and concentrated under reduced pressure. The crude residue was purified by normal phase column chromatography (gradient elution of 0-100% EtOAc/CH₂Cl₂) to afford the protected product as an oil. The oil was dissolved in 1 mL of DCM and 1.0 mL of TFA. The mixture was allowed to stir for 10 min at RT. The reaction mixture was concentrated under reduced pressure. The crude oil was dissolved in DMSO and filtered. The solution was purified by reverse phase column chromatography (gradient elution of 2-55% Acetonitrile/Water+0.1% TFA) to afford 2-45 as a solid. ¹H NMR (500 MHz, DMSO-d₆) δ 9.67 (s, 1H), 7.57 (s, 1H), 7.40 (s, 1H), 6.62 (s, 2H), 4.41-4.35 (m, 2H), 3.53 (tt, J=7.3, 3.9 Hz, 1H), 3.41 (m, 2H), 2.28 (m, 3H), 1.96 (s, 3H), 1.11-1.04 (m, 2H), 1.04-0.98 (m, 2H). MS (EI) calc'd for C₂₃H₂₄FN₈O [M+H]⁺, 447; found, 447.

### Example 2K. Synthesis of Compound 2-46

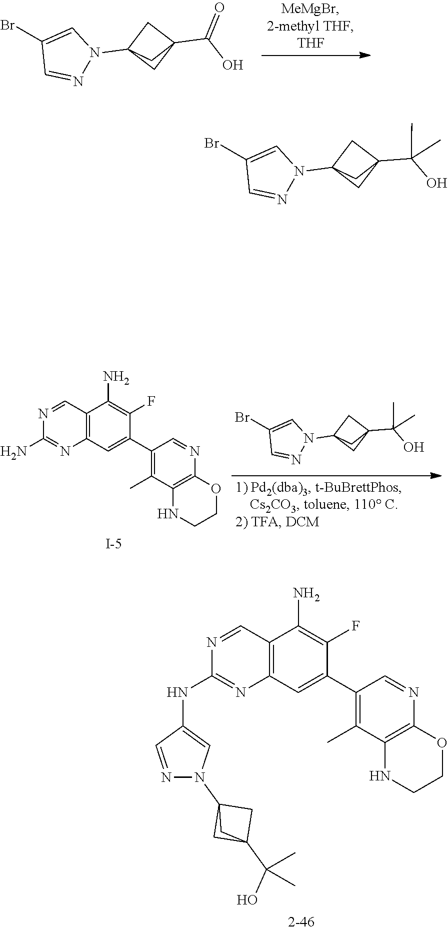

### Step 1. Preparation of 2-(3-(4-bromo-1H-pyrazol-1-yl)bicyclo[1.1.1]pentan-1-yl)propan-2-ol

To a vial containing methyl 3-(4-bromo-1H-pyrazol-1-yl)bicyclo[1.1.1]pentane-1-carboxylate (100 mg, 0.369 mmol) in THF (1.8 mL), stirring at 0° C., was added methylmagnesium bromide (325 μL, 1.11 mmol, 2M in 2-methyl THF). The reaction mixture was allowed to warm to RT and allowed to stir overnight. The reaction mixture was diluted with DCM and quenched with saturated aqueous NH₄C₁. The aqueous layer was extracted with DCM and then with 3:1 CHCl₃:IPA 3×. The combined organic layers were dried over Na₂SO₄, filtered and concentrated under reduced pressure to afford 2-(3-(4-bromo-1H-pyrazol-1-yl)bicyclo[1.1.1]pentan-1-yl)propan-2-ol. MS (EI) calc'd for C₁₁H₁₅BrN₂O [M+H]⁺, 271, 273; found, 271, 273.

### Step 2. Preparation of 2-(3-(4-((5-amino-6-fluoro-7-(8-methyl-2,3-dihydro-1H-pyrido[2,3-b][1,4]oxazin-7-yl)quinazolin-2-yl)amino)-1H-pyrazol-1-yl)bicyclo[1.1.1]pentan-1-yl)propan-2-ol (2-46)

To a vial containing tert-butyl 7-(2-amino-5-(bis(4-methoxybenzyl)amino)-6-fluoroquinazolin-7-yl)-8-methyl-2,3-dihydro-1H-pyrido[2,3-b][1,4]oxazine-1-carboxylate (40 mg, 0.060 mmol) was added 2-(3-(4-bromo-1H-pyrazol-1-yl)bicyclo[1.1.1]pentan-1-yl)propan-2-ol (24.4 mg, 0.0899 mmol), Pd₂(dba)₃ (10.99 mg, 0.012 mmol), t-BuBrettPhos (11.6 mg, 0.0239 mmol) and cesium carbonate (58.6 mg, 0.180 mmol). The mixture was dissolved in toluene (600 μL) and degassed with N₂ for 5 min. The reaction was heated to 100° C. and allowed to stir overnight. The reaction mixture was filtered and concentrated under reduced pressure. The crude residue was purified by normal phase column chromatography (gradient elution of 10-20% MeOH/CH₂Cl₂) to afford the protected product as an oil. The residue was dissolved in 1 mL DCM and 1 mL TFA The reaction mixture was allowed to stir at RT for 1 h. The reaction was concentrated under reduced pressure. The crude residue was dissolved in DMSO and filtered. The solution was purified by reverse phase column chromatography (gradient elution of 15-70% Acetonitrile/Water+0.05% NH₃) to afford 2-46 as a solid. ¹H NMR (500 MHz, DMSO-d) δ 9.57 (s, 1H), 9.50 (s, 1H), 8.16 (s, 1H), 7.58 (s, 1H), 7.35 (s, 1H), 6.65 (s, 1H), 6.28 (s, 2H), 5.68 (s, 1H), 4.33 (s, 1H), 4.29 (m, 2H), 3.38 (m, 2H), 2.51 (p, J=1.7 Hz, 2H), 2.05 (s, 6H), 1.94 (s, 3H), 1.12 (s, 6H). MS (EI) calc'd for C₂₇H₃₀FN₈O₂ [M+H]⁺ 517: found, 517.

Compounds listed in the table below were prepared using the synthetic methods described in the aforementioned examples. The table provides the compound structure, the name, the calculated and observed masses, and the example method used for preparation.

TABLE 2

| | | | Exact |
|---|---|---|---|
| | | | Mass |
| Example | | | [M + H]⁺; |
| Number | Structure | Name | Method |
| 2-1 | 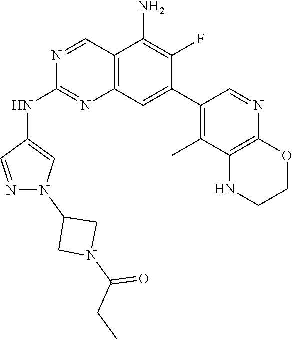 [SMILES: CCC(=O)N1CC(n2cc(Nc3ncc4c(N)c(F)c(-c5cnc6c(c5C)NCCO6)cc4n3)cn2)C1] | 1-[3-(4-{[5-amino-6- fluoro-7-(8-methyl-2,3- dihydro-1H-pyrido[2,3- b][1,4]oxazin-7- yl)quinazolin-2-yl]amino}- 1H-pyrazol-1-yl)azetidin- 1-yl]propan-1-one | Calc'd 504, found 504; 2C |
| 2-2 | 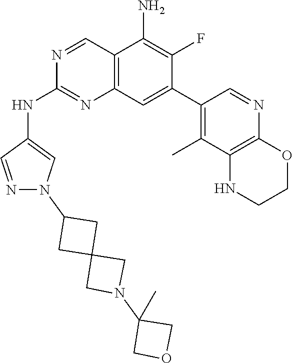 [SMILES: Cc1c(-c2cc3nc(Nc4cnn(C5CC6(C5)CN(C5(C)COC5)C6)c4)ncc3c(N)c2F)cnc2c1NCCO2] | 6-fluoro-7-(8-methyl-2,3- dihydro-1H-pyrido[2,3- b][1,4]oxazin-7-yl)-N~2~- {1-[2-(3-methyloxetan-3- yl)-2-azaspiro[3.3]heptan- 6-yl]-1H-pyrazol-4- yl}quinazoline-2,5-diamine | Calc'd 558, found 558; 2A |
| 2-3 | 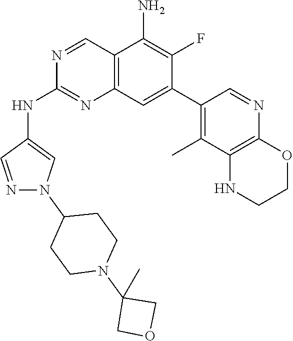 [SMILES: Cc1c(-c2cc3nc(Nc4cnn(C5CCN(C6(C)COC6)CC5)c4)ncc3c(N)c2F)cnc2c1NCCO2] | 6-fluoro-7-(8-methyl-2,3- dihydro-1H-pyrido[2,3- b][1,4]oxazin-7-yl)-N~2~- {1-[1-(3-methyloxetan-3- yl)piperidin-4-yl]-1H- pyrazol-4-yl}quinazoline- 2,5-diamine | Calc'd 546, found 546; 2A |
| 2-4 | 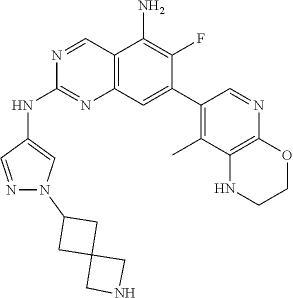 [SMILES: Cc1c(-c2cc3nc(Nc4cnn(C5CC6(CNC6)C5)c4)ncc3c(N)c2F)cnc2c1NCCO2] | N~2~-[1-(2- azaspiro[3.3]heptan-6-yl)- 1H-pyrazol-4-yl]-6-fluoro- 7-(8-methyl-2,3-dihydro- 1H-pyrido[2,3- b][1,4]oxazin-7- yl)quinazoline-2,5-diamine | Calc'd 488, found 488; 2D |
| 2-5 | 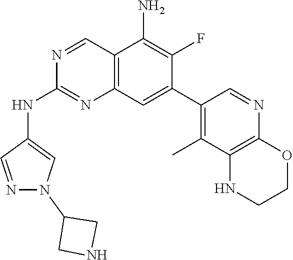 [SMILES: Cc1c(-c2cc3nc(Nc4cnn(C5CNC5)c4)ncc3c(N)c2F)cnc2c1NCCO2] | N~2~-[1-(azetidin-3-yl)- 1H-pyrazol-4-yl]-6-fluoro- 7-(8-methyl-2,3-dihydro- 1H-pyrido[2,3- b][1,4]oxazin-7- yl)quinazoline-2,5-diamine | Calc'd 448, found 448; 2D |
| 2-6 | 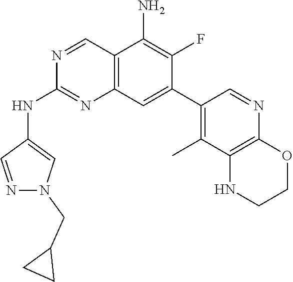 [SMILES: Cc1c(-c2cc3nc(Nc4cnn(CC5CC5)c4)ncc3c(N)c2F)cnc2c1NCCO2] | N~2~-[1- (cyclopropylmethyl)-1H- pyrazol-4-yl]-6-fluoro-7- (8-methyl-2,3-dihydro-1H- pyrido[2,3-b][1,4]oxazin- 7-yl)quinazoline-2,5- diamine | Calc'd 447, found 447; 2C |
| 2-7 | 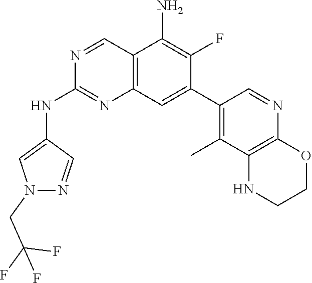 [SMILES: Cc1c(-c2cc3nc(Nc4cnn(CC(F)(F)F)c4)ncc3c(N)c2F)cnc2c1NCCO2] | 6-fluoro-7-(8-methyl-2,3- dihydro-1H-pyrido[2,3- b][1,4]oxazin-7-yl)-N~2~- [1-(2,2,2-trifluoroethyl)- 1H-pyrazol-4- yl]quinazoline-2,5-diamine | Calc'd 475, found 475; 2C |
| 2-8 | 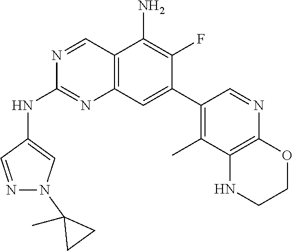 [SMILES: Cc1c(-c2cc3nc(Nc4cnn(C5(C)CC5)c4)ncc3c(N)c2F)cnc2c1NCCO2] | 6-fluoro-N~2~-[1-(1- methylcyclopropyl)-1H- pyrazol-4-yl]-7-(8-methyl- 2,3-dihydro-1H- pyrido[2,3-b][1,4]oxazin- 7-yl)quinazoline-2,5- diamine | Calc'd 447, found 447; 2B |
| 2-9 | 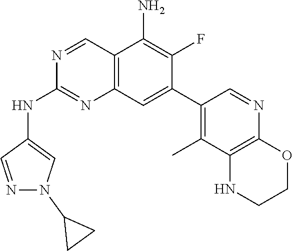 [SMILES: Cc1c(-c2cc3nc(Nc4cnn(C5CC5)c4)ncc3c(N)c2F)cnc2c1NCCO2] | N~2~-(1-cyclopropyl-1H- pyrazol-4-yl)-6-fluoro-7- (8-methyl-2,3-dihydro-1H- pyrido[2,3-b][1,4]oxazin- 7-yl)quinazoline-2,5- diamine | Calc'd 433, found 433; 2B |
| 2-10 | 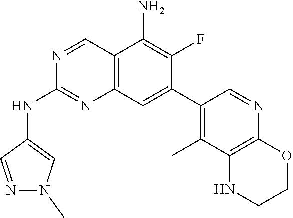 [SMILES: Cc1c(-c2cc3nc(Nc4cnn(C)c4)ncc3c(N)c2F)cnc2c1NCCO2] | 6-fluoro-7-(8-methyl-2,3- dihydro-1H-pyrido[2,3- b][1,4]oxazin-7-yl)-N~2~- (1-methyl-1H-pyrazol-4- yl)quinazoline-2,5-diamine | Calc'd 407, found 407; 2C |
| 2-11 | 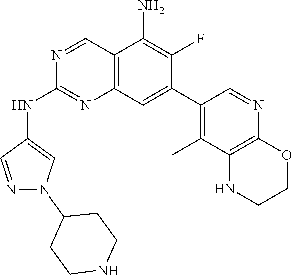 [SMILES: Cc1c(-c2cc3nc(Nc4cnn(C5CCNCC5)c4)ncc3c(N)c2F)cnc2c1NCCO2] | 6-fluoro-7-(8-methyl-2,3- dihydro-1H-pyrido[2,3- b][1,4]oxazin-7-yl)-N~2~- [1-(piperidin-4-yl)-1H- pyrazol-4-yl]quinazoline- 2,5-diamine | Calc'd 476, found 476; 2D |
| 2-12 | 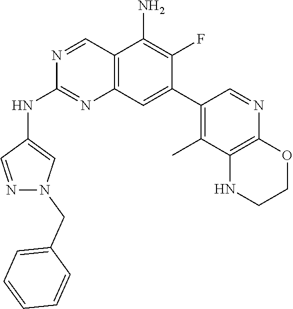 [SMILES: Cc1c(-c2cc3nc(Nc4cnn(Cc5ccccc5)c4)ncc3c(N)c2F)cnc2c1NCCO2] | N~2~-(1-benzyl-1H- pyrazol-4-yl)-6-fluoro-7- (8-methyl-2,3-dihydro-1H- pyrido[2,3-b][1,4]oxazin- 7-yl)quinazoline-2,5- diamine | Calc'd 483, found 483; 2C |
| 2-13 | 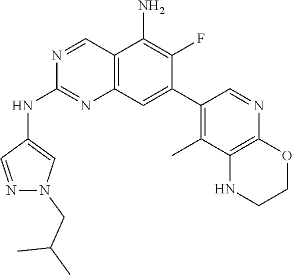 [SMILES: Cc1c(-c2cc3nc(Nc4cnn(CC(C)C)c4)ncc3c(N)c2F)cnc2c1NCCO2] | 6-fluoro-7-(8-methyl-2,3- dihydro-1H-pyrido[2,3- b][1,4]oxazin-7-yl)-N~2~- [1-(2-methylpropyl)-1H- pyrazol-4-yl]quinazoline- 2,5-diamine | Calc'd 449, found 449; 2C |
| 2-14 | 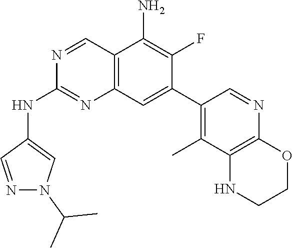 [SMILES: Cc1c(-c2cc3nc(Nc4cnn(C(C)C)c4)ncc3c(N)c2F)cnc2c1NCCO2] | 6-fluoro-7-(8-methyl-2,3- dihydro-1H-pyrido[2,3- b][1,4]oxazin-7-yl)-N~2~- [1-(propan-2-yl)-1H- pyrazol-4-yl]quinazoline- 2,5-diamine | Calc'd 435, found 435; 2C |
| 2-15 | 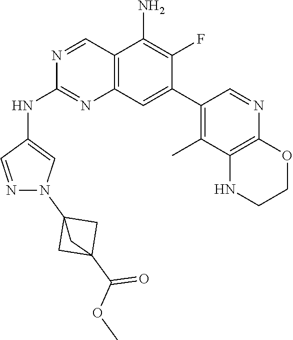 [SMILES: COC(=O)C12CC(n3cc(Nc4ncc5c(N)c(F)c(-c6cnc7c(c6C)NCCO7)cc5n4)cn3)(C1)C2] | methyl 3-(4-{[5-amino-6- fluoro-7-(8-methyl-2,3- dihydro-1H-pyrido[2,3- b][1,4]oxazin-7- yl)quinazolin-2-yl]amino}- 1H-pyrazol-1- yl)bicyclo[1.1.1]pentane-1- carboxylate | Calc'd 517, found 517; 2E |
| 2-16 | 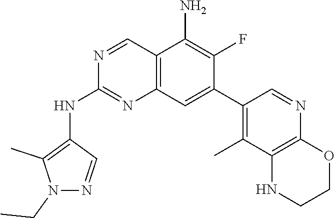 [SMILES: CCn1ncc(Nc2ncc3c(N)c(F)c(-c4cnc5c(c4C)NCCO5)cc3n2)c1C] | N~2~-(1-ethyl-5-methyl- 1H-pyrazol-4-yl)-6-fluoro- 7-(8-methyl-2,3-dihydro- 1H-pyrido[2,3- b][1,4]oxazin-7- yl)quinazoline-2,5-diamine | Calc'd 435, found 435; 2C |
| 2-17 | 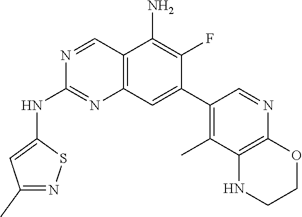 [SMILES: Cc1cc(Nc2ncc3c(N)c(F)c(-c4cnc5c(c4C)NCCO5)cc3n2)sn1] | 6-fluoro-7-(8-methyl-2,3- dihydro-1H-pyrido[2,3- b][1,4]oxazin-7-yl)-N~2~- (3-methyl-1,2-thiazol-5- yl)quinazoline-2,5-diamine | Calc'd 424, found 424; 2C |
| 2-18 | 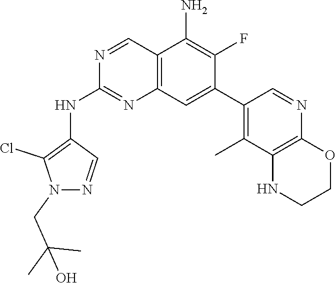 [SMILES: Cc1c(-c2cc3nc(Nc4cnn(CC(C)(C)O)c4Cl)ncc3c(N)c2F)cnc2c1NCCO2] | 1-(4-{[5-amino-6-fluoro-7- (8-methyl-2,3-dihydro-1H- pyrido[2,3-b][1,4]oxazin- 7-yl)quinazolin-2- yl]amino}-5-chloro-1H- pyrazol-1-yl)-2- methypropan-2-ol | Calc'd 499, found 499; 2C |
| 2-19 | 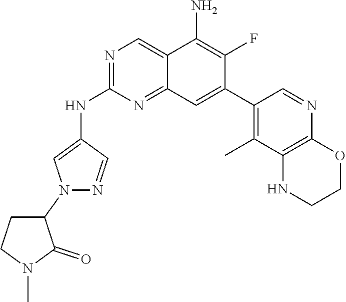 [SMILES: Cc1c(-c2cc3nc(Nc4cnn(C5CCN(C)C5=O)c4)ncc3c(N)c2F)cnc2c1NCCO2] | (S or R)-3-(4-{[5-amino-6- fluoro-7-(8-methyl-2,3- dihydro-1H-pyrido[2,3- b][1,4]oxazin-7- yl)quinazolin-2-yl]amino}- 1H-pyrazol-1-yl)-1- methylpyrrolidin-2-one | Calc'd 490, found 490; 2F |
| 2-20 | 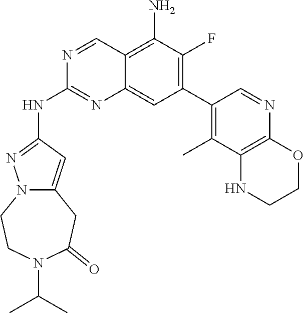 [SMILES: Cc1c(-c2cc3nc(Nc4cc5n(n4)CCN(C(C)C)C(=O)C5)ncc3c(N)c2F)cnc2c1NCCO2] | 2-{[5-amino-6-fluoro-7-(8- methyl-2,3-dihydro-1H- pyrido[2,3-b][1,4]oxazin- 7-yl)quinazolin-2- yl]amino}-6-(propan-2-yl)- 7,8-dihydro-4H- pyrazolo[1,5- d][1,4]diazepin-5(6H)-one | Calc'd 518, found 518; 2C |
| 2-21 | 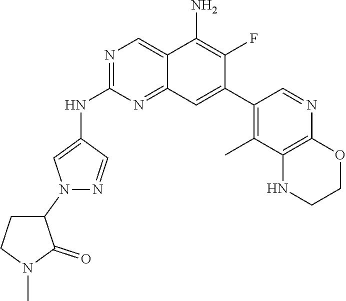 [SMILES: Cc1c(-c2cc3nc(Nc4cnn(C5CCN(C)C5=O)c4)ncc3c(N)c2F)cnc2c1NCCO2] | (S or R)-3-(4-{[5-amino-6- fluoro-7-(8-methyl-2,3- dihydro-1H-pyrido[2,3- b][1,4]oxazin-7- yl)quinazolin-2-yl]amino}- 1H-pyrazol-1-yl)-1- methylpyrrolidin-2-one | Calc'd 490, found 490; 2F |
| 2-22 | 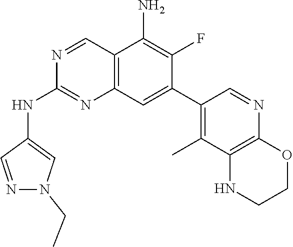 [SMILES: CCn1cc(Nc2ncc3c(N)c(F)c(-c4cnc5c(c4C)NCCO5)cc3n2)cn1] | N~2~-(1-ethyl-1H-pyrazol- 4-yl)-6-fluoro-7-(8-methyl- 2,3-dihydro-1H- pyrido[2,3-b][1,4]oxazin- 7-yl)quinazoline-2,5- diamine | Calc'd 421, found 421; 2C |
| 2-23 | 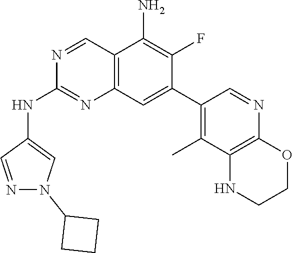 [SMILES: Cc1c(-c2cc3nc(Nc4cnn(C5CCC5)c4)ncc3c(N)c2F)cnc2c1NCCO2] | N~2~-(1-cyclobutyl-1H- pyrazol-4-yl)-6-fluoro-7- (8-methyl-2,3-dihydro-1H- pyrido[2,3-b][1,4]oxazin- 7-yl)quinazoline-2,5- diamine | Calc'd 447, found 447; 2C |
| 2-24 | 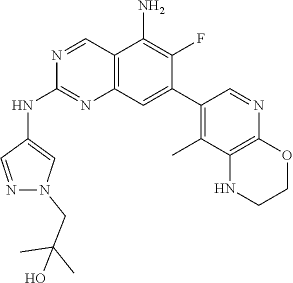 [SMILES: Cc1c(-c2cc3nc(Nc4cnn(CC(C)(C)O)c4)ncc3c(N)c2F)cnc2c1NCCO2] | 1-(4-{[5-amino-6-fluoro-7- (8-methyl-2,3-dihydro-1H- pyrido[2,3-b][1,4]oxazin- 7-yl)quinazolin-2- yl]amino}-1H-pyrazol-1- yl)-2-methylpropan-2-ol | Calc'd 465, found 465; 2C |
| 2-25 | 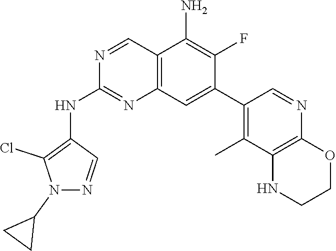 [SMILES: Cc1c(-c2cc3nc(Nc4cnn(C5CC5)c4Cl)ncc3c(N)c2F)cnc2c1NCCO2] | N~2~-(5-chloro-1- cyclopropyl-1H-pyrazol-4- yl)-6-fluoro-7-(8-methyl- 2,3-dihydro-1H- pyrido[2,3-b][1,4]oxazin- 7-yl)quinazoline-2,5- diamine | Calc'd 467, found 467; 2C |
| 2-26 | 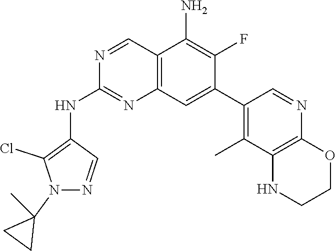 [SMILES: Cc1c(-c2cc3nc(Nc4cnn(C5(C)CC5)c4Cl)ncc3c(N)c2F)cnc2c1NCCO2] | N~2~-[5-chloro-1-(1- methylcyclopropyl)-1H- pyrazol-4-yl]-6-fluoro-7- (8-methyl-2,3-dihydro-1H- pyrido[2,3-b][1,4]oxazin- 7-yl)quinazoline-2,5- diamine | Calc'd 481, found 481; 2C |
| 2-27 | 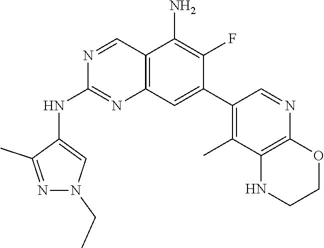 [SMILES: CCn1cc(Nc2ncc3c(N)c(F)c(-c4cnc5c(c4C)NCCO5)cc3n2)c(C)n1] | N~2~-(1-ethyl-3-methyl- 1H-pyrazol-4-yl)-6-fluoro- 7-(8-methyl-2,3-dihydro- 1H-pyrido[2,3- b][1,4]oxazin-7- yl)quinazoline-2,5-diamine | Calc'd 435, found 435; 2C |
| 2-28 | 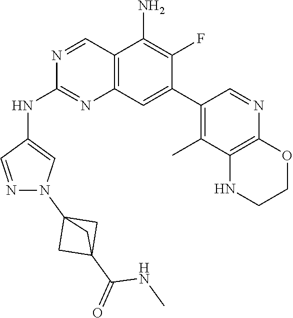 [SMILES: CNC(=O)C12CC(n3cc(Nc4ncc5c(N)c(F)c(-c6cnc7c(c6C)NCCO7)cc5n4)cn3)(C1)C2] | 3-(4-{[5-amino-6-fluoro-7- (8-methyl-2,3-dihydro-1H- pyrido[2,3-b][1,4]oxazin- 7-yl)quinazolin-2- yl]amino}-1H-pyrazol-1- yl)-N- methylbicyclo[1.1.1]pentane- 1-carboxamide | Calc'd 516, found 516; 2I |
| 2-29 | 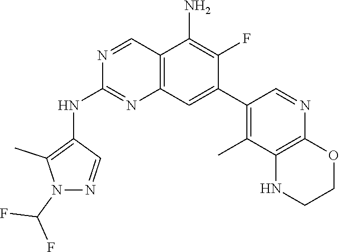 [SMILES: Cc1c(-c2cc3nc(Nc4cnn(C(F)F)c4C)ncc3c(N)c2F)cnc2c1NCCO2] | N~2~-[1-(difluoromethyl)- 5-methyl-1H-pyrazol-4- yl]-6-fluoro-7-(8-methyl- 2,3-dihydro-1H- pyrido[2,3-b][1,4]oxazin- 7-yl)quinazoline-2,5- diamine | Calc'd 457, found 457; 2C |
| 2-30 | 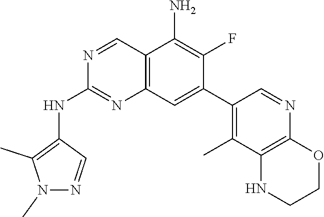 [SMILES: Cc1c(-c2cc3nc(Nc4cnn(C)c4C)ncc3c(N)c2F)cnc2c1NCCO2] | N~2~-(1,5-dimethyl-1H- pyrazol-4-yl)-6-fluoro-7- (8-methyl-2,3-dihydro-1H- pyrido[2,3-b][1,4]oxazin- 7-yl)quinazoline-2,5- diamine | Calc'd 421, found 421; 2C |
| 2-31 | 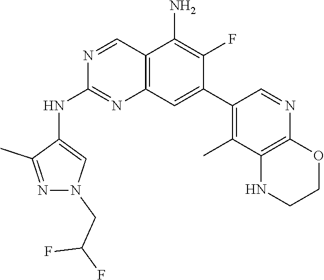 [SMILES: Cc1nn(CC(F)F)cc1Nc1ncc2c(N)c(F)c(-c3cnc4c(c3C)NCCO4)cc2n1] | N~2~-[1-(2,2- difluoroethyl)-3-methyl- 1H-pyrazol-4-yl]-6-fluoro- 7-(8-methyl-2,3-dihydro- 1H-pyrido[2,3- b][1,4]oxazin-7- yl)quinazoline-2,5-diamine | Calc'd 471, found 471; 2C |
| 2-32 | 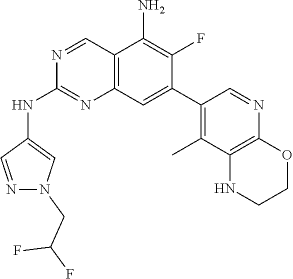 [SMILES: Cc1c(-c2cc3nc(Nc4cnn(CC(F)F)c4)ncc3c(N)c2F)cnc2c1NCCO2] | N~2~-[1-(2,2- difluoroethyl)-1H-pyrazol- 4-yl]-6-fluoro-7-(8-methyl- 2,3-dihydro-1H- pyrido[2,3-b][1,4]oxazin- 7-yl)quinazoline-2,5- diamine | Calc'd 457, found 457; 2C |
| 2-33 | 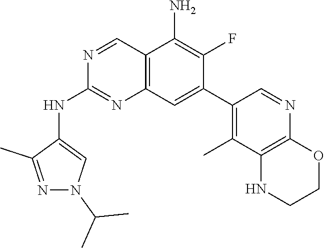 [SMILES: Cc1nn(C(C)C)cc1Nc1ncc2c(N)c(F)c(-c3cnc4c(c3C)NCCO4)cc2n1] | 6-fluoro-7-(8-methyl-2,3- dihydro-1H-pyrido[2,3- b][1,4]oxazin-7-yl)-N~2~- [3-methyl-1-(propan-2-yl)- 1H-pyrazol-4- yl]quinazoline-2,5-diamine | Calc'd 449, found 449; 2C |
| 2-34 | 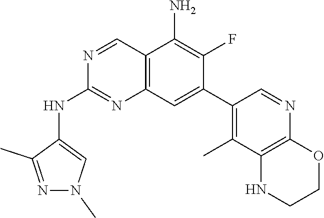 [SMILES: Cc1nn(C)cc1Nc1ncc2c(N)c(F)c(-c3cnc4c(c3C)NCCO4)cc2n1] | N~2~-(1,3-dimethyl-1H- pyrazol-4-yl)-6-fluoro-7- (8-methyl-2,3-dihydro-1H- pyrido[2,3-b][1,4]oxazin- 7-yl)quinazoline-2,5- diamine | Calc'd 421, found 421; 2C |
| 2-35 | 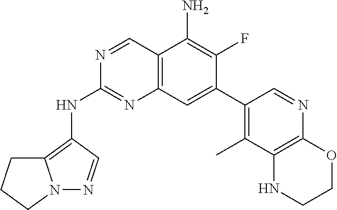 [SMILES: Cc1c(-c2cc3nc(Nc4cnn5c4CCC5)ncc3c(N)c2F)cnc2c1NCCO2] | N~2~-(5,6-dihydro-4H- pyrrolo[1,2-b]pyrazol-3- yl)-6-fluoro-7-(8-methyl- 2,3-dihydro-1H- pyrido[2,3-b][1,4]oxazin- 7-yl)quinazoline-2,5- diamine | Calc'd 433, found 433; 2C |
| 2-36 | 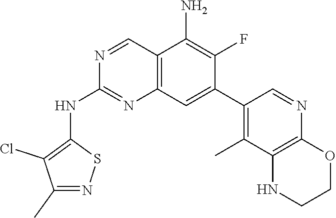 [SMILES: Cc1nsc(Nc2ncc3c(N)c(F)c(-c4cnc5c(c4C)NCCO5)cc3n2)c1Cl] | N~2~-(4-chloro-3-methyl- 1,2-thiazol-5-yl)-6-fluoro- 7-(8-methyl-2,3-dihydro- 1H-pyrido[2,3- b][1,4]oxazin-7- yl)quinazoline-2,5-diamine | Calc'd 458, found 458; 2C |
| 2-37 | 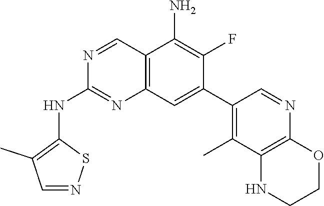 [SMILES: Cc1cnsc1Nc1ncc2c(N)c(F)c(-c3cnc4c(c3C)NCCO4)cc2n1] | 6-fluoro-7-(8-methyl-2,3- dihydro-1H-pyrido[2,3- b][1,4]oxazin-7-yl)-N~2~- (4-methyl-1,2-thiazol-5- yl)quinazoline-2,5-diamine | Calc'd 424, found 424; 2C |
| 2-38 | 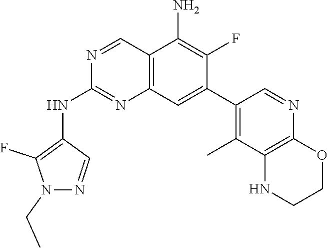 [SMILES: CCn1ncc(Nc2ncc3c(N)c(F)c(-c4cnc5c(c4C)NCCO5)cc3n2)c1F] | N~2~-(1-ethyl-5-fluoro- 1H-pyrazol-4-yl)-6-fluoro- 7-(8-methyl-2,3-dihydro- 1H-pyrido[2,3- b][1,4]oxazin-7- yl)quinazoline-2,5-diamine | Calc'd 439, found 439; 2C |
| 2-39 | 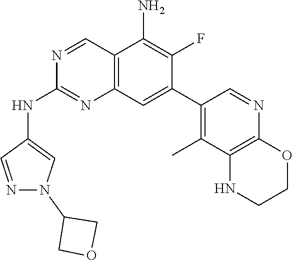 [SMILES: Cc1c(-c2cc3nc(Nc4cnn(C5COC5)c4)ncc3c(N)c2F)cnc2c1NCCO2] | 6-fluoro-7-(8-methyl-2,3- dihydro-1H-pyrido[2,3- b][1,4]oxazin-7-yl)-N~2~- [1-(oxetan-3-yl)-1H- pyrazol-4-yl]quinazoline- 2,5-diamine | Calc'd 449, found 449; 2C |
| 2-40 | 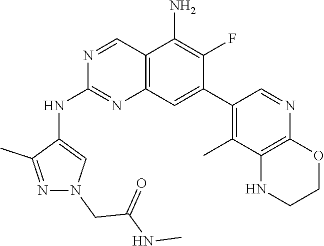 [SMILES: CNC(=O)Cn1cc(Nc2ncc3c(N)c(F)c(-c4cnc5c(c4C)NCCO5)cc3n2)c(C)n1] | 2-(4-{[5-amino-6-fluoro-7- (8-methyl-2,3-dihydro-1H- pyrido[2,3-b][1,4]oxazin- 7-yl)quinazolin-2- yl]amino}-3-methyl-1H- pyrazol-1-yl)-N- methylacetamide | Calc'd 478, found 478; 2I |
| 2-41 | 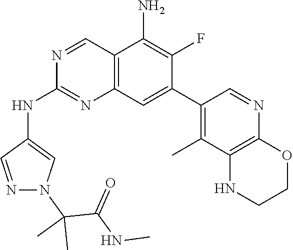 [SMILES: CNC(=O)C(C)(C)n1cc(Nc2ncc3c(N)c(F)c(-c4cnc5c(c4C)NCCO5)cc3n2)cn1] | 2-(4-{[5-amino-6-fluoro-7- (8-methyl-2,3-dihydro-1H- pyrido[2,3-b][1,4]oxazin- 7-yl)quinazolin-2- yl]amino}-1H-pyrazol-1- yl)-N,2- dimethylpropanamide | Calc'd 492, found 492; 2I |
| 2-42 | 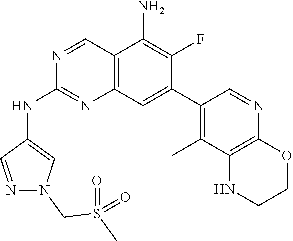 [SMILES: Cc1c(-c2cc3nc(Nc4cnn(CS(C)(=O)=O)c4)ncc3c(N)c2F)cnc2c1NCCO2] | 6-fluoro-7-(8-methyl-2,3- dihydro-1H-pyrido[2,3- b][1,4]oxazin-7-yl)-N2-(1- ((methylsulfonyl)methyl)- 1H-pyrazol-4- y)quinazoline-2,5-diamine | Calc'd 485, found 485; 2C |
| 2-43 | 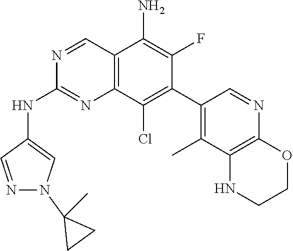 [SMILES: Cc1c(-c2c(F)c(N)c3cnc(Nc4cnn(C5(C)CC5)c4)nc3c2Cl)cnc2c1NCCO2] | (+ and −)-8-chloro-6- fluoro-N~2~-[1-(1- methylcyclopropyl)-1H- pyrazol-4-yl]-7-(8-methyl- 2,3-dihydro-1H- pyrido[2,3-b][1,4]oxazin- 7-yl)quinazoline-2,5- diamine | Calc'd 481 found 481; 2G |
| 2-44 | 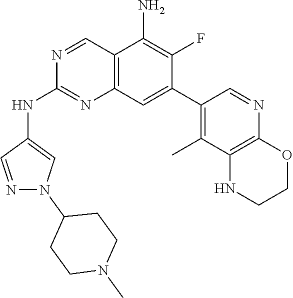 [SMILES: Cc1c(-c2cc3nc(Nc4cnn(C5CCN(C)CC5)c4)ncc3c(N)c2F)cnc2c1NCCO2] | 6-fluoro-7-(8-methyl-2,3- dihydro-1H-pyrido[2,3- b][1,4]oxazin-7-yl)-N~2~- [1-(1-methylpiperidin-4- yl)-1H-pyrazol-4- yl]quinazoline-2,5-diamine | Calc'd 490 found 490; 2H |
| 2-45 | 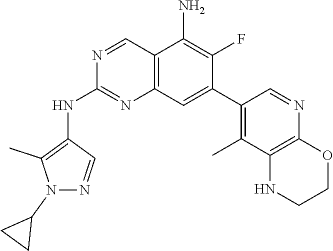 [SMILES: Cc1c(-c2cc3nc(Nc4cnn(C5CC5)c4C)ncc3c(N)c2F)cnc2c1NCCO2] | N~2~-(1-cyclopropyl-5- methyl-1H-pyrazol-4-yl)- 6-fluoro-7-(8-methyl-2,3- dihydro-1H-pyrido[2,3- b][1,4]oxazin-7- yl)quinazoline-2,5-diamine | Calc'd 447 found 447; 2J |
| 2-46 | 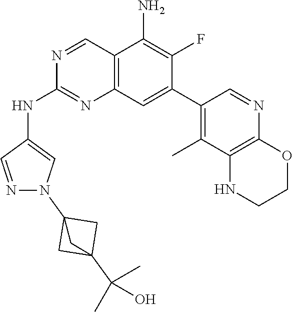 [SMILES: Cc1c(-c2cc3nc(Nc4cnn(C56CC(C(C)(C)O)(C5)C6)c4)ncc3c(N)c2F)cnc2c1NCCO2] | 2-[3-(4-{[5-amino-6- fluoro-7-(8-methyl-2,3- dihydro-1H-pyrido[2,3- b][1,4]oxazin-7- yl)quinazolin-2-yl]amino}- 1H-pyrazol-1- yl)bicyclo[1.1.1]pentan-1- yl]propan-2-ol | Calc'd 517 found 517; 2K |
| 2-47 | 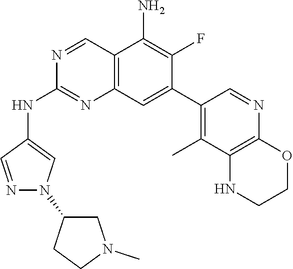 [SMILES: Cc1c(-c2cc3nc(Nc4cnn([C@H]5CCN(C)C5)c4)ncc3c(N)c2F)cnc2c1NCCO2] | (S)-6-fluoro-7-(8-methyl- 2,3-dihydro-1H- pyrido[2,3-b][1,4]oxazin- 7-yl)-N2-(1-(1- methylpyrrolidin-3-yl)-1H- pyrazol-4-yl)quinazoline- 2,5-diamine | Calc'd 476 Found 476; 2H |
| 2-48 | 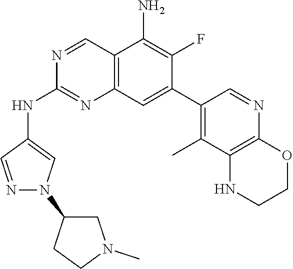 [SMILES: Cc1c(-c2cc3nc(Nc4cnn([C@@H]5CCN(C)C5)c4)ncc3c(N)c2F)cnc2c1NCCO2] | (R)-6-fluoro-7-(8-methyl- 2,3-dihydro-1H- pyrido[2,3-b][1,4]oxazin- 7-yl)-N2-(1-(1- methylpyrrolidin-3-yl)-1H- pyrazol-4-yl)quinazoline- 2,5-diamine | Calc'd 476 Found 476; 2H |

### HPK1-SLP76 TR-FRET ASSAY

### Assay Principle:

HPK1-Catalytic domain enzyme is preincubated for 30 minutes with varying concentrations of investigational test compounds, or DMSO reference. HPK1 activity is initiated by the addition of ATP and results in phosphorylation of a His-tagged SLP-76 protein substrate. Following a 60-minute reaction time, the reaction is quenched and FRET partners Eu-anti-His Ab and phospho-SLP-76 (Ser376) (D7S1K) XP Rabbit mAb (AF 647 Conjugate) are added to detect the phosphorylated His-tagged SLP-76 product.

### Instrumentation:

Labcyte Echo
Beckman Coulter BioRaptr
Perkin Elmer Envision

### Final Assay Conditions:

HPK1-Kinase Domain: 0.075 nM
Full length SLP-76: 10 nM
ATP: 10 μM
Eu-anti-His Tag: 0.75 nM
pSLP76(Ser376)-AF647: 0.75 nM
Preincubation Time: 30 minutes
Kinase Reaction Time: 60 minutes
Temperature: Ambient Room Temperature
Reaction Volume: 7.5 μL
Total Volume: 10.0 μL

### Materials:

Assay Plate: Black Corning NBS 384-well microplate #3820
Kinase: Human HPK1, catalytic domain [1-346 amino acids of accession number NP_009112.1]
Substrate: Full Length SLP76, C-Terminal 8×His-tag
ATP: 100 mM
BSA: 10%
Reaction Buffer: 50 mM HEPES (pH 7.5), 10 mM MgCl₂, 1 mM EGTA, 0.01% Brij-35, 0.05% BSA and 0.5 mM TCEP.
Detection Solution: Reaction Buffer with LANCE Eu-W1024 Anti-6×His Ab, Phospho-SLP-76 (Ser376) (D7S1K) XP Rabbit mAb (AF 647 Conjugate) (Cell Signaling Technologies) and 10 mM EDTA.

### General Assay Procedure:

To each well of black Corning #3820 384-well plate, an ECHO was used to dispense 7.5 nL of DMSO or Test compound in DMSO. A 1.5× kinase solution, 5 μL/well, was added and preincubated for 30 minutes before 2.5 μL/well of 3× substrate solution was added. The reaction solution incubated for 60 minutes and quenched with 2.5 μL of 4× detection solution. The solutions were incubated for an additional 60 minutes prior to reading on a Perkin Elmer Envision. The TR-FRET signal was measured at both 615 and 665 nm. The calculated emission ratio of 665/615 was used to determine the percent effect for each compound concentration.

### Detailed HPK1 Assay Protocol:

Compounds are serially diluted (3-fold in 100% DMSO) across a 384-well polypropylene source plated from column 3 to column 12 and column 13 to column 22, to yield 10 concentration dose responses for each test compound. Columns 1, 2, 23 and 24 contain either only DMSO or a pharmacological known control inhibitor. Once titrations are made, 7.5 nL of the compounds on 384 well plates are transferred by acoustic dispersion into a 384-well assay plate (Corning 3820) to assay the HPK1 enzyme.

The HPK1 kinase biochemical assay was developed using commercially available HTRF reagents. The assay contains the following reagents: 1) Assay Buffer: 50 mM HEPES (pH 7.5), 10 mM MgCl₂, 1 mM EGTA, 0.01% Brij-35, 0.05% BSA and 0.5 mM TCEP; 2) Enzyme Solution: HPK1 (Cama); 3) Substrate Solution: ATP and Full Length SLP76 with His-Tag; 4) Stop and Detection Solution: EDTA, LANCE Eu-W1024 Anti-6×His Ab (Perkin Elmer) and Phospho-SLP-76 (Ser376) (D7S1K) XP Rabbit mAb (AF 647 Conjugate) (Cell Signaling Technologies).

Enzyme, Substrate and Stop/Detection solutions are prepared in assay buffer. Enzyme solution (75 μM HPK1 Final), 5 μL/well, is added to 384-well assay plate and incubated with 7.5 nL of compound or DMSO for 30 minutes. Kinase reaction is initiated with addition of 2.5 μL of substrate solution (ATP 10 uM and SLP76 10 nM Final) and allowed to proceed for 60 minutes.

Enzyme addition and compound pre-incubation are initiated by the addition of 5 μL of HPK1 enzyme solution (at one and a half times its final concentration of 75 μM) to all wells using a BioRaptr. Plates are incubated at room temperature for 30 minutes. Reactions are initiated by addition of 2.5 μL of 3× substrate solution (10 nM SLP76 and 10 uM ATP Final) using BioRaptr. Plates are incubated at room temperature for one hour. Reactions are quenched, and activity detected by addition of 2.5 μL of 4× stop and detection solution (10 mM EDTA, 0.75 nM LANCE Eu-W1024 Anti-6×His Ab and 0.75 nM Phospho-SLP-76 (Ser376) (D7S1K) XP Rabbit mAb (AF 647 Conjugate) Final) to all wells using the BioRaptr. Following a one-hour incubation, the HTRF signal is measured on the Envision plate reader set for 320 nm excitation and dual emission detection at 615 nM (Eu) and 665 nM (AF647).

### Data Analysis:

The loss of the HTRF signal is due to the inhibition of HPK1 activity and decreased phosphorylation of SLP76 substrate. All data were calculated using the ratio of acceptor (AF647) to donor (Europium) fluorescence in each well of the assay plate. The percent effect for each compound concentration was calculated as follows: % Effect=100×(Emission ratio−Minimum Effect Control)/(Maximum Effect Control−Minimum Effect Control) where Minimum Effect Control=HPK1+DMSO and Maximum Effect Control=HPK1 Kinase reaction+known reference inhibitor. An EC₅₀ was then calculated fitting the % effect data. Dose response data were analyzed using the 4 parameter logistic nonlinear regression model; Y=Bottom+(Top−Bottom)/(1+10\hat{ }((Log(EC₅₀)−X)*Hill Slope)). Where: Y=% E (percent effect) described above; X=base 10 logarithm of molar drug concentration; Bottom=lower limit of dose response (minimum % E); Top=upper limit of dose response (maximum % E); EC₅₀=concentration at which 50%; effect is achieved; Hill Slope=Hill slope coefficient; slope of curve at EC₅₀.

### HPK1-SLP76 TR-FRET ASSAY DATA

The following table tabulates the biological data disclosed for the current invention. The biological data was collected using the methodology described above. For each compound, HPK1 IC₅₀ values are listed in nanomolar (nM) concentration units.

| Example | IC50 (nM) |
|---|---|
| 1-1 | 1.2 |
| 1-2 | 0.22 |
| 1-3 | 0.26 |
| 1-4 | 5.3 |
| 1-5 | 0.71 |
| 1-6 | 42 |
| 1-7 | 0.34 |
| 1-8 | 0.6747 |
| 2-1 | 0.42 |
| 2-2 | 0.14 |
| 2-3 | 0.12 |
| 2-4 | 0.47 |
| 2-5 | 0.29 |
| 2-6 | 0.14 |
| 2-7 | 0.26 |
| 2-8 | 0.088 |
| 2-9 | 0.14 |
| 2-10 | 0.13 |
| 2-11 | 0.071 |
| 2-12 | 2.8 |
| 2-13 | 0.77 |
| 2-14 | 0.16 |
| 2-15 | 0.22 |
| 2-16 | 0.51 |
| 2-17 | 0.38 |
| 2-18 | 2.0 |
| 2-19 | 0.15 |
| 2-20 | 0.063 |
| 2-21 | 0.35 |
| 2-22 | 0.076 |
| 2-23 | 0.19 |
| 2-24 | 1.1 |
| 2-25 | 0.49 |
| 2-26 | 0.38 |
| 2-27 | 1.0 |
| 2-28 | 0.094 |
| 2-29 | 0.10 |
| 2-30 | 0.051 |
| 2-31 | 0.20 |
| 2-32 | 0.054 |
| 2-33 | 3.5 |
| 2-34 | 0.058 |
| 2-35 | 0.051 |
| 2-36 | 0.28 |
| 2-37 | 0.071 |
| 2-38 | 0.57 |
| 2-39 | 0.051 |
| 2-40 | 0.27 |
| 2-41 | 0.84 |
| 2-42 | 0.13 |
| 2-43 | 0.14 |
| 2-44 | 0.045 |
| 2-45 | 0.18 |
| 2-46 | 0.051 |
| 2-47 | 0.1768 |
| 2-48 | 0.1658 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of the formula (I): 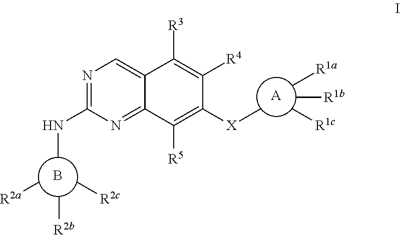
wherein:
A is a phenyl, cycloalkyl, or heterocyclyl ring;
B is a pyrazolyl or thiazolyl ring;
X is a bond, —O—, —O(C₁₋₃alkyl)-, —NH—, —NH(C₁₋₃alkyl)- or —N(CH₃)(C₁₋₃alkyl)-;
R^{1a}, R^{1b} and R^{1c} as are present are independently selected from:
(1) hydrogen,
(2) halogen,
(3) hydroxyl,
(4) C₁₋₆alkyl, which is unsubstituted or substituted with substituents selected from: hydroxy, —NH₂, —(CO)NH(C₁₋₆alkyl), —CN, and fluoro,
(5) —O—C₁₋₆alkyl, which is unsubstituted or substituted with fluoro,
(6) —C₃₋₆cyclolkyl, which is unsubstituted or substituted with substituents selected from: hydroxy, methoxy, —(CO)NH(C₁₋₆alkyl), —(CO)O(C₁₋₆alkyl), —CN, and fluoro,
(7) C₂₋₆alkynyl,
(8) —NH₂,
(9) —NH(C₁₋₆alkyl),
(10) —N(C₁₋₆alkyl)₂,
(11) —(CO)(C₁₋₆alkyl),
(12) —(CO)NH₂,
(13) —(CO)NH(C₁₋₆alkyl),
(14) —(CO)NH(C₃₋₆cycloalkyl),
(15) —NH(CO)(C₁₋₆alkyl),
(16) —SO₂—C₁₋₆alkyl,
(17) —NH—SO₂—C₁₋₆alkyl,
(18) —CN,
(19) keto,
(20) -phenyl,
(21) -pyridyl,
(22) -diazolyl,
(23) -morpholinyl,
(24) -oxazolyl,
(25) -oxadiazolyl,
(26) -piperazinyl,
(27) -piperidinyl, and
(28) -thiazolyl,
or R^{1a} and R^{1b} may be joined to form a 1,4-dioxanyl, 1,3-dioxolanyl, morpholinyl, oxazolyl, piperidinyl, pyrazolyl, pyrrolidinyl, tetrahydropyranyl, tetrahydroquinolinyl, or thiazolyl ring;
R^{2a}, R^{2b} and R^{2c} as are present are independently selected from:
(1) hydrogen,
(2) halogen,
(3) hydroxyl,
(4) C₁₋₆alkyl, which is unsubstituted or substituted with substituents selected from: hydroxy, —(CO)NH(C₁₋₆alkyl), —(CO)O(C₁₋₆alkyl), —SO₂—C₁₋₆alkyl, C₃₋₆cycloalkyl, —CN, phenyl and fluoro,
(5) —O—C₁₋₆alkyl, which is unsubstituted or substituted with fluoro,
(6) —C₃₋₆cycloalkyl, which is unsubstituted or substituted with substituents selected from: hydroxy, methoxy, —C₁₋₆alkyl, —(CO)NH(C₁₋₆alkyl), —(CO)O(C₁₋₆alkyl), —CN, and fluoro,
(7) —NH₂,
(8) —NH(C₁₋₆alkyl),
(9) —N(C₁₋₆alkyl)₂,
(10) —(CO)(C₁₋₆alkyl),
(11) —(CO)NH₂,
(12) —(CO)NH(C₁₋₆alkyl),
(13) —NH(CO)(C₁₋₆alkyl),
(14) —SO₂—C₁₋₆alkyl,
(15) —SO₂—NH(C₁₋₆alkyl),
(16) —SO₂—N(C₁₋₆alkyl)₂,
(17) azaspiro[3.3]heptanyl, which is unsubstituted or substituted with —C₁₋₆alkyl or oxetanyl, which is unsubstituted or substituted with —C₁₋₆alkyl,
(18) azetidinyl, which is unsubstituted or substituted with —C₁₋₆alkyl or —(CO)O(C₁₋₆alkyl),
(19) bicyclopentyl, which is unsubstituted or substituted with —C₁₋₆alkyl or —(CO)O(C₁₋₆alkyl),
(20) oxazolyl,
(21) oxadiazolyl,
(22) oxetanyl, which is unsubstituted or substituted with —C₁₋₆alkyl,
(23) piperidinyl, which is unsubstituted or substituted with —C₁₋₆alkyl or oxetanyl, which is unsubstituted or substituted with —C₁₋₆alkyl,
(24) pyrrolidinyl, which is unsubstituted or substituted with oxo or —C₁₋₆alkyl, and
(25) thiazolyl,
or R^{2b} and R^{2c} may be joined to form a pyrrolyl, dihydrospiro[1,4′-pyrazolo[1,5-d][1,4]diazepin]-7′(8′H)-one, or dihydro-4H-pyrazolo[1,5-d][1,4]diazepin-one ring, which is unsubstituted or substituted with —C₁₋₆alkyl, —C₁₋₆alkyl-OH or —C₃₋₆cycloalkyl;
R³ is selected from:
(1) hydrogen,
(2) —NH₂,
(3) chloro, and
(4) fluoro;
R⁴ is selected from:
(1) hydrogen,
(2) cyano,
(3) chloro, and
(4) fluoro;
R⁵ is selected from:
(1) hydrogen,
(2) methyl,
(3) cyano,
(4) chloro,
(5) fluoro, and
(6) bromo;
or a pharmaceutically acceptable salt thereof.

2. The compound of claim 1, of the formula II: 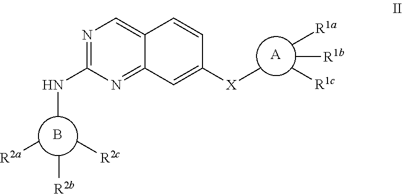
or a pharmaceutically acceptable salt thereof.

3. The compound of claim 2, or a pharmaceutically acceptable salt thereof, wherein A is a phenyl, pyridyl, or 2,3-dihydro-1H-pyrido[2,3-b][1,4]oxazin-7-yl ring.

4. The compound of claim 3, or a pharmaceutically acceptable salt thereof, wherein B is a pyazol-4-yl ring.

5. The compound of claim 4, or a pharmaceutically acceptable salt thereof, wherein X is a bond.

6. The compound of claim 5, or a pharmaceutically acceptable salt thereof, wherein R^{1a}, R^{1b} and R^{1c} as are present are independently selected from:
(1) hydrogen,
(2) fluoro,
(3) hydroxyl,
(4) —CH₃,
(5) —CHF₂,
(6) —CF₃,
(7) —CH₂OH,
(8) —CH₂CH₃,
(9) —C(CH₃)OH,
(10) —OCH₃,
(11) —OCHF₂,
(12) —OCH₂CH₂F,
(13) —N(CH₃)₂,
(14) cyclopropyl, and
(15) phenyl.

7. The compound of claim 6, or a pharmaceutically acceptable salt thereof, wherein R^{2a}, R^{2b} and R^{2c} as are present are independently selected from:
(1) hydrogen,
(2) halogen,
(3) hydroxyl,
(4) C₁₋₆alkyl, and
(5) —O—C₁₋₆alkyl.

8. The compound of claim 7, or a pharmaceutically acceptable salt thereof, wherein R³ is hydrogen or —NH₂.

9. The compound of claim 1 8, or a pharmaceutically acceptable salt thereof, wherein R⁴ is hydrogen or fluoro.

10. The compound of claim 9, or a pharmaceutically acceptable salt thereof, wherein R³ is hydrogen, R⁴ is hydrogen and R⁵ is hydrogen.

11. The compound of claim 1 9, or a pharmaceutically acceptable salt thereof, wherein R³ is —NH₂, R⁴ is fluoro and R⁵ is hydrogen.

12. A compound which is selected from the group consisting of:
7-(8-methyl-2,3-dihydro-1H-pyrido[2,3-b][1,4]oxazin-7-yl)-N-[1-(oxetan-3-yl)-1H-pyrazol-4-yl]quinazolin-2-amine;
7-(8-methyl-2,3-dihydro-1H-pyrido[2,3-b][1,4]oxazin-7-yl)-N-{1-[2-(3-methyloxetan-3-yl)-2-azaspiro[3.3]heptan-6-yl]-1H-pyrazol-4-yl}quinazolin-2-amine;
1-[3-(4-{[7-(8-methyl-2,3-dihydro-1H-pyrido[2,3-b][1,4]oxazin-7-yl)quinazolin-2-yl]amino}-1H-pyrazol-1-yl)azetidin-1-yl]propan-1-one;
6′-methyl-2′-{[7-(8-methyl-2,3-dihydro-1H-pyrido[2,3-b][1,4]oxazin-7-yl)quinazolin-2-yl]amino}-5′,6′-dihydrospiro[cyclopropane-1,4′-pyrazolo[1,5-d][1,4]diazepin]-7′(8′H)-one;
7-(8-methyl-2,3-dihydro-1H-pyrido[2,3-b][1,4]oxazin-7-yl)-N-(1-methyl-1H-pyrazol-4-yl)quinazolin-2-amine;
2-{[7-(8-methyl-2,3-dihydro-1H-pyrido[2,3-b][1,4]oxazin-7-yl)quinazolin-2-yl]amino}-6-(propan-2-yl)-5,6-dihydro-4H-pyrazolo[1,5-d][1,4]diazepin-7(8H)-one;
2-{[7-(8-methyl-2,3-dihydro-1H-pyrido[2,3-b][1,4]oxazin-7-yl)quinazolin-2-yl]amino}-6-(propan-2-yl)-7,8-dihydro-4H-pyrazolo[1,5-d][1,4]diazepin-5(6H)-one;
6-(2-hydroxy-2-methylpropyl)-2-((7-(8-methyl-2,3-dihydro-1H-pyrido[2,3-b][1,4]-oxazin-7-yl)quinazolin-2-yl)amino)-7,8-dihydro-4H-pyrazolo[1,5-d][1,4]diazepin-5(6H)-one;
1-[3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)azetidin-1-yl]propan-1-one;
6-fluoro-7-(8-methyl-2,3-dihydro-1H-pyrido[2,3-b][1,4]oxazin-7-yl)-N˜2˜-{1-[2-(3-methyloxetan-3-yl)-2-azaspiro[3.3]heptan-6-yl]-1H-pyrazol-4-yl}quinazoline-2,5-diamine;
6-fluoro-7-(8-methyl-2,3-dihydro-1H-pyrido[2,3-b][1,4]oxazin-7-yl)-N˜2˜-{1-[1-(3-methyloxetan-3-yl)piperidin-4-yl]-1H-pyrazol-4-yl}quinazoline-2,5-diamine;
N˜2˜-[1-(2-azaspiro[3.3]heptan-6-yl)-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-[1-(azetidin-3-yl)-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-[1-(cyclopropylmethyl)-1H-pyrazol-4-yl]-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[1-(2,2,2-trifluoroethyl)-1H-pyrazol-4-yl]quinazoline-2,5-diamine;
6-fluoro-N˜2˜-[1-(1-methylcyclopropyl)-1H-pyrazol-4-yl]-7-(8-methyl-2,3-dihydro-1H-pyrido[2,3-b][1,4]oxazin-7-yl)quinazoline-2,5-diamine;
N˜2˜-(1-cyclopropyl-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-(1-methyl-1H-pyrazol-4-yl)quinazoline-2,5-diamine;
6-fluoro-7-(8-methyl-2,3-dihydro-1H-pyrido[2,3-b][1,4]oxazin-7-yl)-N˜2˜-[1-(piperidin-4-yl)-1H-pyrazol-4-yl]quinazoline-2,5-diamine;
N˜2˜-(1-benzyl-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[1-(2-methylpropyl)-1H-pyrazol-4-yl]quinazoline-2,5-diamine;
6-fluoro-7-(8-methyl-2,3-dihydro-1H-pyrido[2,3-b][1,4]oxazin-7-yl)-N˜2˜-[1-(propan-2-yl)-1H-pyrazol-4-yl]quinazoline-2,5-diamine;
methyl 3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)bicyclo[1.1.1]pentane-1-carboxylate;
N˜2˜-(1-ethyl-5-methyl-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-(3-methyl-1,2-thiazol-5-yl)quinazoline-2,5-diamine;
1-(4-{[5-amino-6-fluoro-7-(8-methyl-2,3-dihydro-1H-pyrido[2,3-b][1,4]oxazin-7-yl)quinazolin-2-yl]amino}-5-chloro-1H-pyrazol-1-yl)-2-methylpropan-2-ol;
3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-1-methylpyrrolidin-2-one;
2-{[5-amino-6-fluoro-7-(8-methyl-2,3-dihydro-1H-pyrido[2,3-b][1,4]oxazin-7-yl)quin-azolin-2-yl]amino}-6-(propan-2-yl)-7,8-dihydro-4H-pyrazolo[1,5-d][1,4]diazepin-5(6H)-one;
3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-1-methylpyrrolidin-2-one;
N˜2˜-(1-ethyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-(1-cyclobutyl-1H-pyrazol-4-yl)-6-fluoro-7-(8-methyl-2,3-dihydro-1H-pyrido[2,3-b][1,4]oxazin-7-yl)quinazoline-2,5-diamine;
1-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-2-methylpropan-2-ol;
N˜2˜-(5-chloro-1-cyclopropyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-[5-chloro-1-(1-methylcyclopropyl)-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-(1-ethyl-3-methyl-1H-pyrazol-4-yl)-6-fluoro-7-(8-methyl-2,3-dihydro-1H-pyrido[2,3-b][1,4]oxazin-7-yl)quinazoline-2,5-diamine;
3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-N-methylbicyclo[1.1.1]pentane-1-carboxamide;
N˜2˜-[1-(difluoromethyl)-5-methyl-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-(1,5-dimethyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-[1-(2,2-difluoroethyl)-3-methyl-1H-pyrazol-4-yl]-6-fluoro-7-(8-methyl-2,3-dihydro-1H-pyrido[2,3-b][1,4]oxazin-7-yl)quinazoline-2,5-diamine;
N˜2˜-[1-(2,2-difluoroethyl)-1H-pyrazol-4-yl]-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[3-methyl-1-(propan-2-yl)-1H-pyrazol-4-yl]quinazoline-2,5-diamine;
N˜2˜-(1,3-dimethyl-1H-pyrazol-4-yl)-6-fluoro-7-(8-methyl-2,3-dihydro-1H-pyrido[2,3-b][1,4]oxazin-7-yl)quinazoline-2,5-diamine;
N˜2˜-(5,6-dihydro-4H-pyrrolo[1,2-b]pyrazol-3-yl)-6-fluoro-7-(8-methyl-2,3-dihydro-1H-pyrido[2,3-b][1,4]oxazin-7-yl)quinazoline-2,5-diamine;
N˜2˜-(4-chloro-3-methyl-1,2-thiazol-5-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-(4-methyl-1,2-thiazol-5-yl)quinazoline-2,5-diamine;
N˜2˜-(1-ethyl-5-fluoro-1H-pyrazol-4-yl)-6-fluoro-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[1-(oxetan-3-yl)-1H-pyrazol-4-yl]quinazoline-2,5-diamine;
2-(4-{[5-amino-6-fluoro-7-(8-methyl-2,3-dihydro-1H-pyrido[2,3-b][1,4]oxazin-7-yl)quinazolin-2-yl]amino}-3-methyl-1H-pyrazol-1-yl)-N-methylacetamide;
2-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)-N,2-dimethylpropanamide;
6-fluoro-7-(8-methyl-2,3-dihydro-1H-pyrido[2,3-b][1,4]oxazin-7-yl)-N2-(1-((methylsulfonyl)methyl)-1H-pyrazol-4-yl)quinazoline-2,5-diamine;
8-chloro-6-fluoro-N˜2˜-[1-(1-methylcyclopropyl)-1H-pyrazol-4-yl]-7-(8-methyl-2,3-dihydro-1H-pyrido[2,3-b][1,4]oxazin-7-yl)quinazoline-2,5-diamine;
6-fluoro-7-(8-methyl-2,3-dihydro-1H-pyrido[2,3-b][1,4]oxazin-7-yl)-N˜2˜-[1-(1-methylpiperidin-4-yl)-1H-pyrazol-4-yl]quinazoline-2,5-diamine;
N˜2˜-(1-cyclopropyl-5-methyl-1H-pyrazol-4-yl)-6-fluoro-7-(8-methyl-2,3-dihydro-1H-pyrido[2,3-b][1,4]oxazin-7-yl)quinazoline-2,5-diamine;
2-[3-(4-{[5-amino-6-fluoro-7-(8-methyl-2,3-dihydro-1H-pyrido[2,3-b][1,4]oxazin-7-yl)quinazolin-2-yl]amino}-1H-pyrazol-1-yl)bicyclo[1.1.1]pentan-1-yl]propan-2-ol;
(S)-6-fluoro-7-(8-methyl-2,3-dihydro-1H-pyrido[2,3-b][1,4]oxazin-7-yl)-N2-(1-(1-methylpyrrolidin-3-yl)-1H-pyrazol-4-yl)quinazoline-2,5-diamine; and
(R)-6-fluoro-7-(8-methyl-2,3-dihydro-1H-pyrido[2,3-b][1,4]oxazin-7-yl)-N2-(1-(1-methylpyrrolidin-3-yl)-1H-pyrazol-4-yl)quinazoline-2,5-diamine;
or a pharmaceutically acceptable salt thereof.

13. A pharmaceutical composition which comprises an inert carrier and a compound of claim 1, or a pharmaceutically acceptable salt thereof.

14. (canceled)

15. (canceled)

16. A method for treating an HPK1-associated disease or disorder in a mammalian subject which comprises administering to the subject an effective amount of a compound of claim 1, or a pharmaceutically acceptable salt thereof.

17. A method for treating an HPK1-associated disease or disorder in a mammalian subject which comprises administering to the subject an effective amount of a compound of claim 1, or a pharmaceutically acceptable salt thereof in combination with another anti-cancer agent.

18. The method of claim 16 wherein the HPK1-associated disease or disorder is a cancer, metastasis, inflammation and auto-immune pathogenesis.

19. The method of claim 18, wherein the cancer is liposarcoma, neuroblastoma, glioblastoma, bladder cancer, adrenocortical cancer, multiple myeloma, colorectal cancer, non-small cell lung cancer, oropharyngeal cancer, penis cancer, anal cancer, thyroid cancer, vaginal cancer, gastric cancer, rectal cancer, thyroid cancer, Hodgkin lymphoma or diffuse large B-cell lymphoma.
